SEQUENCE LISTING (7 sequences)

SEQ ID NO: 1 - protein, 609 aa, Homo sapiens [sequence omitted]

SEQ ID NO: 2 - protein, 148 aa, Homo sapiens
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 3 - protein, 245 aa, Streptococcus suis
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 280 aa, Staphlyococcus
METLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGLNKDKIVYDRTNINYNMVLQGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 5 - protein, 495 aa, Staphylococcus
MAKTQAEINKRLDAYAKGTVDSPYRIKKATSYDPSFGVMEAGAIDADGYYHAQCQDLITDYVLWLTDNKVRTWGNAKDQIKQSYGTGFKIHENKPSTVPKKGWIAVFTSGSYQQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNHIPGVAWGVFK

SEQ ID NO: 6 - protein, 495 aa, Staphylococcus
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKKTAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 7 - protein, 493 aa, Staphylococcus simulans
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

(19) United States
(12) Patent Application Publication
(10) Pub. No.: US 2022/0023399 A1
(43) Pub. Date: Jan. 27, 2022

(54) BLOOD COMPONENT POTENTIATION OF LYTIC PROTEIN ANTI-BACTERIAL ACTIVITY AND METHODS AND USES THEREOF

(71) Applicant: CONTRAFECT CORPORATION, Yonkers, NY (US)

(72) Inventor: Raymond SCHUCH, Mountain Lakes, NJ (US)

(21) Appl. No.: 16/629,916

(22) PCT Filed: Jul. 10, 2018
(86) PCT No.: PCT/US2018/041498

§ 371 Date: Jan. 9, 2020

Related U.S. Application Data
(60) Provisional application No. 62/530,632, filed on Jul. 10, 2017.

(51) Int. Cl. A61K 38/48; A61K 38/38; A61K 38/47; A61K 38/54; A61K 31/201; A61K 31/20; A61P 31/04; C12N 9/50; C12N 9/96; C07K 14/765; A61K 38/14; A61K 38/12; C12Q 1/14; C12Q 1/18

(52) U.S. Cl. CPC A61K 38/4886; C07K 2319/31; A61K 38/385; A61K 38/47; A61K 38/54; C12Y 302/01017; A61K 31/201; A61K 31/20; A61P 31/04; C12N 9/503; C12N 9/96; C07K 14/765; A61K 38/14; A61K 38/12; C12Q 1/14; C12Q 1/18; C12Y 304/24075

(57) ABSTRACT

The present invention provides methods, assays, compositions, formulations, and constructs, particularly lytic peptide constructs, which relate to and are based on the activity and use of blood components, particularly serum albumin and lysozyme, and their activity and use to enhance or synergize with the bacterial killing effect of anti-bacterial lytic proteins and peptides.

### CROSS-REFERENCE TO RELATED APPLICATIONS

This application is a U.S. National Stage Application of International Application No. PCT/US2018/041498, filed on Jul. 10, 2018, which claims priority to U.S. Provisional Patent Application No. 62/530,632, filed on Jul. 10, 2017, the entire disclosures of which are hereby incorporated by reference in their entireties.

### FIELD OF THE INVENTION

The present invention relates generally to blood components, particularly serum albumin and lysozyme, and their activity and use to enhance or synergize with the bacterial killing effect of anti-bacterial lytic proteins and peptides. The invention further relates to lytic peptide constructs, formulations, assays and methods based on the blood components and/or peptides or proteins thereof.

### BACKGROUND OF THE INVENTION

Gram-positive bacteria are surrounded by a cell wall containing polypeptides and polysaccharide. The gram-positive cell wall appears as a broad, dense wall that is 20-80 nm thick and consists of numerous interconnecting layers of peptidoglycan. Between 60% and 90% of the gram-positive cell wall is peptidoglycan, providing cell shape, a rigid structure, and resistance to osmotic shock. The cell wall does not exclude the Gram stain crystal violet, allowing cells to be stained purple, and therefore “Gram-positive.” Gram-positive bacteria include but are not limited to the genera Actinomyces, Bacillus, Listeria, Lactococcus, Staphylococcus, Streptococcus, Enterococcus, Mycobacterium, Corynebacterium, and Clostridium. Medically relevant species include Streptococcus pyogenes, Streptococcus pneumoniae, Staphylococcus aureus, and Enterococcus faecalis. Bacillus species, which are spore-forming, cause anthrax and gastroenteritis. Spore-forming Clostridium species are responsible for botulism, tetanus, gas gangrene and pseudomembranous colitis. Corynebacterium species cause diphtheria, and Listeria species cause meningitis.

Antibacterials that inhibit cell wall synthesis, such as penicillins and cephalosporins, interfere with the linking of the interpeptides of peptidoglycan and weaken the cell wall of both gram positive and gram negative bacteria. Because the peptidoglycans of gram-positive bacteria are exposed, gram-positive bacteria are more susceptible to these antibiotics. Advantageously, eukaryotic cells lack cell walls and are not susceptible to these drugs or other cell wall agents.

The development of drug resistant, particularly antibiotic resistant, bacteria is a major problem in medicine as more antibiotics are used for a wide variety of illnesses and other conditions. Novel antimicrobial therapy approaches include enzyme-based antibiotics (“enzybiotics”) such as bacteriophage lysins. Phages use these lysins to digest the cell wall of their bacterial hosts, releasing viral progeny through hypotonic lysis. The high lethal activity of lysins against gram-positive pathogens makes them attractive candidates for development as therapeutics (Fischetti, V. A. (2008) Curr Opinion Microbiol 11:393-400; Nelson, D. L. et al (2001) Proc Natl Acad Sci USA 98:4107-4112). Bacteriophage lysins were initially proposed for eradicating the nasopharyngeal carriage of pathogenic streptococci (Loeffler, J. M. et al (2001) Science 294: 2170-2172; Nelson, D. et al (2001) Proc Natl Acad Sci USA 98:4107-4112).

Bacteriophage lytic enzymes have been established as useful in the assessment and specific treatment of various types of infection in subjects through various routes of administration. For example, U.S. Pat. No. 5,604,109 (Fischetti et al.) relates to the rapid detection of Group A streptococci in clinical specimens, through the enzymatic digestion by a semi-purified Group C streptococcal phage associated lysin enzyme. This enzyme work became the basis of additional research, leading to methods of treating diseases. Fischetti and Loomis patents (U.S. Pat. Nos. 5,985,271, 6,017,528 and 6,056,955) disclose the use of a lysin enzyme produced by group C streptococcal bacteria infected with a C1 bacteriophage. U.S. Pat. No. 6,248,324 (Fischetti and Loomis) discloses a composition for dermatological infections by the use of a lytic enzyme in a carrier suitable for topical application to dermal tissues. U.S. Pat. No. 6,254,866 (Fischetti and Loomis) discloses a method for treatment of bacterial infections of the digestive tract which comprises administering a lytic enzyme specific for the infecting bacteria. U.S. Pat. No. 6,264,945 (Fischetti and Loomis) discloses a method and composition for the treatment of bacterial infections by the parenteral introduction (intramuscularly, subcutaneously, or intravenously) of at least one lytic enzyme produced by a bacteria infected with a bacteriophage specific for that bacteria and an appropriate carrier for delivering the lytic enzyme into a patient.

U.S. Pat. Nos. 7,402,309 and 8,580,553, 7,638,600 and 8,389,469 provide distinct phage-associated lytic enzymes, PlyG, Gamma and W, and PlyPH respectively, useful as antibacterial agents for treatment or reduction of Bacillus anthracis infections. U.S. Pat. No. 7,569,223 describes Pal lytic enzymes for Streptococcus pneumoniae. Lysin useful for Enterococcus (E. faecalis and E. faecium, including vancomycin resistant strains), particularly PlyV12, are described in U.S. Pat. No. 7,582,291. U.S. Pat. No. 8,105,585 describes mutant PlyGBS lysins highly effective in killing Group B streptococci. A chimeric lysin denoted ClyS, with activity against Staphylococci bacteria, including Staphylococcus aureus, is detailed in WO 2010/002959 and U.S. Pat. No. 8,840,900. PlySs2 lysin, isolated from Streptococcus suis and effective in killing Streptococcus, Staphylococcus, Enterococcus and Listeria strains, is described in WO2012/145630 and U.S. Pat. No. 9,034,322.

PlySs2 lysin (also denoted CF-301, CF301, PlySs2/CF-301, PlySs2 (CF-301) herein) is the first lysin to enter into and complete FDA-allowed Phase I clinical trials. PlySs2 lysin is described in U.S. Pat. No. 9,034,322 and PCT Application PCT/US2012/34456, and also in Gilmer et al (Gilmer D B et al (2013) Antimicrob Agents Chemother Epub 2013 Apr. 9 [PMID 23571534]). PlySs2 (CF-301) lysin may be combined with standard of care antibiotics (including but not limited to, vancomycin or daptomycin) to treat bloodstream infections, including endocarditis, caused by methicillin-sensitive and -resistant Staphylococcus aureus.

In support of clinical trials, in vitro antibiotic susceptibility testing (AST) is utilized to evaluate and standardize the bacterial agent(s). Broth microdilution (BMD) can be used to test lysin such as PlySs2 (CF-301) activity against S. aureus isolates, however the standard method (CLSI methodology) is not a dependable assay and demonstrates various problems when applied to a lytic polypeptide such as PlySs2 (CF-301). PlySs2 (CF-301) is more effective inhuman blood, serum and plasma than in artificial media. An understanding of the enhanced activity and functionality of lysin such as PlySs2 (CF-301) in human blood, serum and plasma may provide novel, useful and improved antibacterial approaches, methods, and therapeutics.

The citation of references herein shall not be construed as an admission that such is prior art to the present invention.

### SUMMARY OF THE INVENTION

In a general aspect, the invention relates to identification and characterization of an additional and novel ability of lysins, particularly lysin(s) polypeptides having an SH3-type binding domain including PlySs2 (CF-301) lysin, to interact with latent antimicrobial factors in human blood to potentiate bacteriolysis. The invention relates to the unique property of lysin polypeptides, particularly lysin(s) polypeptides having an SH3-type binding domain including PlySs2 (CF-301) lysin, to synergize with and provide activation of blood components which have no or limited intrinsic antibacterial activity, particularly antistaphylococcal activity, of their own, thus allowing for maximal bacteriocidal activity.

The present application relates to activity enhancing effects of blood component proteins, particularly serum albumin and lysozyme, particularly human serum albumin and human lysozyme, and antibacterial lytic peptides, particularly lysins. In one aspect, serum albumin is selected from human serum albumin, rabbit serum albumin, dog serum albumin and horse serum albumin. Serum albumin, particularly human serum albumin, enhances or otherwise facilitates the antibacterial activity of lysin polypeptide, particularly a lysin polypeptide having an SH-3 type binding domain, such as selected from PlySs2 (CF-301) lysin, Sal lysin, LysK lysin, lysostaphin, phill lysin, LysH5 lysin, MV-L lysin, LysGH15 lysin, and ALE-1 lysin, particularly PlySs2 (CF-301) lysin. In one aspect lysozyme is human lysozyme. Lysozyme, particularly human lysozyme, serves to enhance or otherwise facilitate the antibacterial activity of lysin polypeptide, particularly PlySs2 (CF-301) lysin. In an aspect, combinations of lysin polypeptide, particularly PlySs2 (CF-301) lysin polypeptide, and human lysozyme, act synergistically to kill gram-positive bacteria.

In an aspect of the invention, a combination of lysin polypeptide(s) and one or more blood component protein is provided. In an aspect of the invention, a combination of lysin polypeptide(s) and one or more blood component protein selected from serum albumin and lysozyme is provided. In one aspect a combination of lysin polypeptide having an SH3-type binding domain and one or more blood component protein selected from serum albumin and lysozyme is provided. In a particular aspect a composition or combination comprising a lysin polypeptide having an SH3-type binding domain and selected from PlySs2 (CF-301) lysin, Sal lysin, LysK lysin, lysostaphin, phill lysin, LysH5 lysin, MV-L lysin, LysGH15 lysin, and ALE-1 lysin, or effective variants thereof capable of binding gram-positive bacteria, including Staphylococcus, and one or more blood component protein selected from serum albumin and lysozyme is provided. In a particular aspect, a composition or combination comprising a lysin polypeptide having an SH3-type binding domain and selected from PlySs2 (CF-301) lysin, Sal lysin, LysK lysin, lysostaphin, or effective variants thereof capable of binding gram-positive bacteria, including Staphylococcus, and one or more blood component protein selected from serum albumin and lysozyme is provided.

In an aspect of the invention, a synergistic combination of lysin polypeptide(s) and one or more blood component protein is provided, wherein the one or more blood component has no or limited intrinsic antibacterial activity in the absence of the lysin polypeptide(s). In an aspect of the invention, the combination of lysin polypeptide(s) and one or more blood component protein has synergistic killing activity against gram positive bacteria, particularly Staphylococcus bacteria. In an aspect of the invention, a synergistic combination of lysin polypeptide(s) and one or more blood component protein selected from serum albumin and lysozyme is provided. In one aspect a synergistic combination of lysin polypeptide having an SH3-type binding domain and one or more blood component protein selected from serum albumin and lysozyme is provided. In a particular aspect, a composition or synergistic combination comprising a lysin polypeptide having an SH3-type binding domain and selected from PlySs2 (CF-301) lysin, Sal lysin, LysK lysin, lysostaphin, or effective variants thereof capable of binding gram-positive bacteria, including Staphylococcus, and one or more blood component protein selected from serum albumin and lysozyme is provided. In an aspect, the composition or combination further includes one or more serum fatty acid, such as selected from oleate and palmitate.

In an aspect of the invention, lysozyme, particularly human lysozyme, is effective against Staphylococcus aureus bacteria when combined with lysin polypeptide PlySs2 (CF-301). In an aspect of the invention, lysozyme, particularly human lysozyme, is rendered effective against Staphylococcus aureus bacteria when combined with or otherwise in the presence of lysin polypeptide. In an aspect, Staphylococcus aureus bacteria is sensitive to lysozyme, particularly human lysozyme, when combined with or otherwise in the presence of lysin polypeptide PlySs2 (CF-301).

In an aspect of the invention, lysozyme, particularly human lysozyme, is rendered effective against Staphylococcus aureus bacteria when combined with or otherwise in the presence of lysin polypeptide having an SH3-type binding domain. In an aspect of the invention, serum albumin, particularly human serum albumin, enhances or otherwise facilitates the antibacterial activity of lysin polypeptide having an SH3-type binding domain.

In a particular aspect of the invention, lysin polypeptide having an SH3-type binding domain is selected from PlySs2 (CF-301) lysin, Sal lysin, LysK lysin, lysostaphin, or effective variants thereof capable of binding gram-positive bacteria, including Staphylococcus. In a particular aspect of the invention, lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, ALE-1 lysin, or effective variants thereof capable of binding gram-positive bacteria, including Staphylococcus.

The present application relates to modified methods and assays utilizing blood components for determining the minimal inhibitory concentration and assessing antibacterial killing effectiveness of peptides, particularly anti-bacterially effective peptides, particularly lytic peptides. The application provides methods and assays for determining the minimal inhibitory concentration and assessing antibacterial killing effectiveness of peptides, particularly anti-bacterially effective peptides, particularly lytic peptides, wherein serum albumin and/or lysozyme are added in order to accurately determine and/or predict the antibacterial killing effectiveness and/or minimal inhibitory concentration in an animal or in vivo, particularly in a human, of anti-bacterially effective peptides, particularly lytic peptides, including PlySs2 (CF-301). In an aspect, human lysozyme is added. In an aspect human serum albumin is added. In an aspect serum albumin from or corresponding in sequence to serum albumin of a human, rabbit, dog, or horse is added. In an aspect, both serum albumin and lysozyme are added.

The invention relates to a system or assay for determining MIC or evaluating or quantitating antibacterial activity and/or effectiveness of an antibacterial peptide, particularly a lytic peptide, wherein the assay is conducted utilizing assay solution, broth or media supplemented with serum albumin. The invention relates to a system or assay for determining MIC or evaluating or quantitating antibacterial activity and/or effectiveness of an antibacterial peptide, particularly a lytic peptide, wherein the assay is conducted utilizing assay solution, broth or media supplemented with lysozyme. The invention relates to a system or assay for determining MIC or evaluating or quantitating antibacterial activity and/or effectiveness of an antibacterial peptide, particularly a lytic peptide, wherein the assay is conducted utilizing assay solution, broth or media supplemented with serum albumin and lysozyme. In an aspect of the invention, an assay is provided for determining bacterial killing effectiveness of an antibacterial peptide that accurately reflects the bacterial killing effectiveness of an antibacterial peptide, such as a lytic peptide or lysin, in a mammal or patient, particularly a human.

In an aspect, the lysozyme is human lysozyme. In an aspect, the serum albumin is human, rabbit, dog, horse, rat or calf (cow/bovine) serum albumin or corresponds in amino acid sequence to human, rabbit, dog, horse, rat or calf (cow/bovine) serum albumin. In an aspect, the serum albumin is human, rabbit, dog, or horse serum albumin or corresponds in amino acid sequence to human, rabbit, dog, or horse serum albumin. In an aspect, the serum albumin is homologous to natural human, rabbit, dog, or horse serum albumin and differs in amino acid sequence from natural human, rabbit, dog, or horse serum albumin by one or more amino acid sequence. The serum albumin or lysozyme of use in the invention may be purified or recombinant. The serum albumin or lysozyme may be full length protein or a peptide or fragment thereof, wherein said peptide or fragment thereof demonstrates activity of the full length protein with regard to lysin polypeptide activity enhancing effects and/or with regard to lysin polypeptide binding capability. The serum albumin or lysozyme may be full length protein or a peptide or fragment thereof, wherein said peptide or fragment thereof demonstrates increased activity compared with the full length protein with regard to lysin polypeptide activity enhancing effects and/or with regard to lysin polypeptide binding capability. In an aspect, the serum albumin or peptide or fragment thereof amino acid sequence is distinct from natural sequence. In an aspect, the serum albumin or peptide or fragment thereof amino acid sequence is distinct from natural sequence and has greater enhancing activity or improved lysin polypeptide binding activity.

In accordance with the invention, a method is provided for determining bacterial killing activity of an antibacterial peptide, such as a lytic polypeptide or lysin, wherein the killing activity accurately mimics the bacterial killing of said antibacterial peptide in a human, comprising evaluating an antibacterial peptide in broth, assay medium or solution supplemented with serum albumin isolated from or corresponding to human serum albumin, rabbit serum albumin, dog serum albumin or home serum albumin, or an effective peptide or fragment thereof. In an aspect of the invention a lytic polypeptide or lysin, is evaluated against susceptible bacteria in solution, media or broth supplemented with human serum albumin, horse serum albumin, dog serum albumin, or rabbit serum albumin. In an aspect, a reducing agent is additionally added to the broth, assay medium or solution. In an aspect, the reducing agent is DL-Dithiothreitol (DTT. In an aspect, the reducing agent is Tris(2-carboxyethyl)phosphine hydrochloride (TCEP). In an aspect, solution, media or broth is supplemented with 0.5 mM DL-Dithiothreitol (DTT).

In accordance with the invention a modified and improved broth microdilution (BMD) method and assay is provided for testing peptides, particularly anti-bacterially effective peptides, particularly lytic peptides or lysin peptides. In an aspect of the invention, a modified BMD is provided that utilizes broth or media for evaluation, wherein the broth or media is supplemented with serum albumin, particularly human serum albumin, horse serum albumin, dog serum albumin, or rabbit serum albumin. In an aspect of the invention, a modified BMD is provided that utilizes broth or media for evaluation, wherein the broth or media is supplemented with lysozyme, particularly human lysozyme. In an aspect of the invention, a modified BMD is provided that utilizes broth or media for evaluation, wherein the broth or media is supplemented with serum albumin, particularly human serum albumin, horse serum albumin, dog serum albumin, or rabbit serum albumin, and lysozyme, particularly human lysozyme.

The amount of serum albumin for supplementation may be determined by comparison to human serum. In an aspect, the amount of serum albumin supplemented is comparable to the amount ordinarily present in a sample of human blood or serum.

The amount of lysozyme for supplementation may be determined by comparison to the ordinary amount of lysozyme present in a human sample. In an aspect, the amount of lysozyme supplemented is comparable to the amount ordinarily present in a sample of human blood or serum.

In an aspect, the amount of reducing agent is between 0.1 mM and 10 mM. In an aspect, the amount of reducing agent is between 0.1 mM and 5 mM. In an aspect, the amount of reducing agent is between 0.1 mM and 2 mM. In an aspect, the amount of reducing agent is between 0.1 mM and 1 mM. In an aspect, the amount of reducing agent is between 0.1 mM and 0.9 mM. In an aspect, the amount of reducing agent is between 0.1 mM and 0.6 mM. In an aspect, the amount of reducing agent is between 0.2 mM and 0.6 mM. In an aspect, the amount of reducing agent is between 0.3 mM and 0.6 mM. In an aspect, the amount of reducing agent is between 0.4 mM and 0.6 mM. In an aspect, the amount of reducing agent is about 0.5 mM. In an aspect, the amount of reducing agent is between 0.25 mM and 1 mM. In an aspect, the amount of reducing agent is less than 1 mM.

In an embodiment, the assay and method of the invention is used in the assessment and analysis of a lytic polypeptide. In an aspect of the invention, the BMD method with supplement(s) is utilized in determining the bacterial killing effectiveness of a lytic polypeptide active against Streptococcus bacteria. In an aspect of the invention, the BMD method with supplement(s) is utilized in determining the bacterial killing effectiveness of a lytic polypeptide active against Streptococcus and Staphylococcus bacteria. In an aspect of the invention, the BMD method with supplement(s) is utilized in MIC testing of a lytic polypeptide active against Streptococcus bacteria. In an aspect of the invention, the BMD method with supplement(s) is utilized in MIC testing of a lytic polypeptide active against Staphylococcus bacteria. In an aspect of the invention, the BMD method with supplement(s) is utilized in MIC testing of a lytic polypeptide active against Streptococcus and Staphylococcus bacteria. In an aspect of the invention, the BMD method with supplement(s) is utilized in MIC testing of a lytic polypeptide active against Enterococcus bacteria. In an aspect of the invention, the BMD method with supplement(s) is utilized in MIC testing of a lytic polypeptide against gram positive bacteria. In an aspect of the invention, the BMD method with supplement(s) is utilized in MIC testing of a lytic polypeptide against more than one species of gram positive bacteria. The gram positive bacteria may be selected from Streptococcus, Staphylococcus, Enterococcus and Listeria bacteria. The gram positive bacteria may be antibiotic resistant bacteria or antibiotic sensitive bacteria.

In accordance with the invention, novel or modified formulations such as effective antibacterial compositions are provided comprising a lysin polypeptide, particularly a lysin polypeptide having an SH3-type binding domain, and one or more of serum albumin, particularly human serum albumin, rabbit serum albumin, dog serum albumin, horse serum albumin, rat serum albumin, calf (cow/bovine) serum albumin, or an effective or binding peptide or fragment thereof, and/or lysozyme, particularly human lysozyme, or an effective peptide or fragment thereof. In accordance with an aspect of the invention, novel or modified formulations such as effective antibacterial compositions are provided comprising a lysin polypeptide, particularly a lysin polypeptide having an SH3-type binding domain, and one or more of serum albumin, particularly human serum albumin, rabbit serum albumin, dog serum albumin or horse serum albumin, or an effective or binding peptide or fragment thereof, and/or lysozyme, particularly human lysozyme, or an effective peptide or fragment thereof. In an aspect, effective antibacterial compositions are provided comprising a lysin polypeptide, particularly a lysin polypeptide selected from PlySs2 (CF-301) lysin, Sal lysin, LysK lysin, lysostaphin, phill lysin, LysH5 lysin, MV-L lysin, LysGH15 lysin, ALE-1 lysin, or effective variants thereof capable of binding and/or killing gram-positive bacteria, particularly Staphylococcus or Streptococcus, or Enterococcus bacteria, and one or more of serum albumin, particularly human serum albumin, rabbit serum albumin, dog serum albumin, horse serum albumin, rat serum albumin, or calf (cow/bovine) serum albumin, or an effective or binding peptide or fragment thereof, or lysozyme, particularly human lysozyme, or an effective peptide or fragment thereof. In another aspect, effective antibacterial compositions are provided comprising a lysin polypeptide, particularly a lysin polypeptide selected from PlySs2 (CF-301) lysin, Sal lysin, LysK lysin, lysostaphin, phill lysin, LysH5 lysin, MV-L lysin, LysGH15 lysin, ALE-1 lysin, or effective variants thereof capable of binding and/or killing gram-positive bacteria, particularly Staphylococcus or Streptococcus, or Enterococcus bacteria, and one or more of serum albumin, particularly human serum albumin, rabbit serum albumin, dog serum albumin or horse serum albumin, or an effective or binding peptide or fragment thereof, or lysozyme, particularly human lysozyme, or an effective peptide or fragment thereof. In an aspect, an antibacterial composition is provided comprising PlySs2 (CF-301) lysin, or a variant thereof effective to bind and/or kill Staphylococcus and Streptococcus bacteria, and serum albumin, particularly human serum albumin, rabbit serum albumin, dog serum albumin or horse serum albumin. In an aspect, an antibacterial composition is provided comprising PlySs2 (CF-301) lysin, or a variant thereof effective to bind and/or kill Staphylococcus and Streptococcus bacteria, and lysozyme, particularly human lysozyme. In an aspect, the formulation is a topical or inhalable formulation. In an aspect, the composition is formulated for effectiveness against a skin infection or against a lung infection or oral infection.

In an aspect, a topical formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and one or more of serum albumin, particularly human serum albumin, rabbit serum albumin, dog serum albumin, horse serum albumin, rat serum albumin, or calf (cow/bovine) serum albumin, or an effective or binding peptide or fragment thereof. In another aspect, a topical formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and one or more of serum albumin, particularly human serum albumin, rabbit serum albumin, dog serum albumin or horse serum albumin, or an effective or binding peptide or fragment thereof. In an aspect, a topical formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and lysozyme, particularly human lysozyme, or an effective peptide or fragment thereof. In an aspect, an inhalable or orally administered formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and one or more of serum albumin, particularly human serum albumin, rabbit serum albumin, dog serum albumin, horse serum albumin, rat serum albumin, or calf (cow/bovine) serum albumin, or an effective or binding peptide or fragment thereof. In another aspect, an inhalable or orally administered formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and one or more of serum albumin, particularly human serum albumin, rabbit serum albumin, dog serum albumin or horse serum albumin, or an effective or binding peptide or fragment thereof. In an aspect, an inhalable or orally administered formulation of a composition is provided for treating gram-positive bacteria skin infections, particularly Staphylococcal or Streptococcal bacteria skin infections, comprising a lysin polypeptide, particularly a lysin polypeptide having an SH3-type binding domain, and lysozyme, particularly human lysozyme, or an effective peptide or fragment thereof. In a particular aspect, the topical or inhalable formulation is for treating Staphylococcus aureus infection. In a particular aspect, the topical or inhalable formulation is for treating antibiotic resistant Staphylococcus aureus infection. In an aspect, the lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, and ALE-1 lysin. In an aspect, the lysin polypeptide having an SH3-type binding domain is PlySs2 lysin or an effective variant thereof.

The amount of serum albumin in a formulation or composition may be determined by comparison to human serum. In an aspect, the amount of serum albumin is comparable to the amount ordinarily present in a sample of human blood or serum.

The amount of lysozyme in a formulation or composition may be determined by comparison to the ordinary amount of lysozyme present in a human sample. In an aspect, the amount of lysozyme is comparable to the amount ordinarily present in a sample of human blood or serum.

A further aspect of the invention relates to chimeric, dimeric or fusion peptides or lysins comprising an SH3 binding domain operatively linked to or fused to serum albumin, particularly human serum albumin, rabbit serum albumin, dog serum albumin or horse serum albumin, or operatively linked to or fused to lysozyme, particularly human lysozyme. In an aspect, the chimeric, dimeric or fusion peptides or lysins comprise at least one catalytic domain and an SH3 binding domain operatively linked to or fused to serum albumin or lysozyme, particularly human serum albumin or human lysozyme or an active or binding fragment or peptide thereof. In an aspect, chimeric, dimeric or fusion peptide comprising an SH3 binding domain operatively linked to or fused to serum albumin, particularly human serum albumin, rabbit serum albumin, dog serum albumin, horse serum albumin, rat serum albumin, or calf (cow/bovine) serum albumin, including a bacterial binding fragment or peptide thereof may be utilized to deliver a therapeutic agent, drug or other payload effectively to gram-positive bacteria, including Staphylococcus or Streptococcus bacteria. In another aspect, chimeric, dimeric or fusion peptide comprising an SH3 binding domain operatively linked to or fused to serum albumin, particularly human serum albumin, rabbit serum albumin, dog serum albumin or horse serum albumin, including a bacterial binding fragment or peptide thereof, may be utilized to deliver a therapeutic agent, drug or other payload effectively to gram-positive bacteria, including Staphylococcus or Streptococcus bacteria. In an aspect, the therapeutic agent, drug or other payload can be one or more of lysozyme or other antibacterial peptide, including a chimeric or dimeric lysin. The therapeutic agent, drug or other payload may be covalently bound to, fused to or operably linked with the SH3 binding domain. The therapeutic agent, drug or other payload may be capable of binding to serum albumin and rendered an aspect or payload by virtue of serum albumin binding or affinity.

In an aspect, the invention provides modified lysin polypeptides operatively linked or fused to peptides or bacterial binding and lysin binding fragments of serum albumin, particularly human serum albumin, rabbit serum albumin, dog serum albumin, horse serum albumin, rat serum albumin, or calf (cow/bovine) serum albumin. In another aspect, the invention provides modified lysin polypeptides operatively linked or fused to peptides or bacterial binding and lysin binding fragments of serum albumin, particularly human serum albumin, rabbit serum albumin, dog serum albumin or horse serum albumin. In one aspect lysin PlySs2 or an effective variant thereof, is fused or covalently attached to serum albumin, particularly human serum albumin, rabbit serum albumin, dog serum albumin, horse serum albumin, rat serum albumin, calf (cow/bovine) serum albumin, or a peptide or fragment of serum albumin, wherein said peptide or fragment is capable of binding bacterial cells, including Staphylococcus or Streptococcus bacteria. In one aspect lysin PlySs2 or an effective variant thereof, is fused or covalently attached to serum albumin, particularly human serum albumin, rabbit serum albumin, dog serum albumin or horse serum albumin, or a peptide or fragment of serum albumin, wherein said peptide or fragment is capable of binding bacterial cells, including Staphylococcus or Streptococcus bacteria. In one aspect the lysin polypeptide is lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, and ALE-1 lysin. In an aspect, the lysin polypeptide is PlySs2 (CF-301).

In an aspect, the invention provides modified lysin polypeptides operatively linked or fused to peptides or bacterial binding and lysin binding fragments of lysozyme, particularly human lysozyme. In one aspect, lysin polypeptide selected from PlySs2 (CF-301) lysin, Sal lysin, LysK lysin, lysostaphin, phill lysin, LysH5 lysin, MV-L lysin, LysGH15 lysin, and ALE-1 lysin or an effective killing variant thereof, is fused or covalently attached to lysozyme, particularly human lysozyme, or a fragment of lysozyme capable of cleaving gram-positive bacteria peptidoglycan. In one aspect, lysin PlySs2 (CF-301) or an effective variant thereof capable of killing Staphylococcus and Streptococcus bacteria, is fused or covalently attached to lysozyme, particularly human lysozyme, or a fragment of lysozyme capable of cleaving gram-positive bacteria peptidoglycan.

In accordance with the invention, methods are provided for killing gram-positive bacteria comprising the step of contacting the bacteria with a composition comprising an amount of an isolated lysin polypeptide having an SH3-type binding domain effective to kill gram-positive bacteria, further contacting the bacteria with serum albumin, particularly human serum albumin, and/or lysozyme, particularly human lysozyme. In an aspect, methods are provided for reducing a population of gram-positive bacteria comprising the step of contacting the bacteria with a composition comprising an amount of an isolated lysin polypeptide having an SH3-type binding domain effective to kill gram-positive bacteria, further contacting the bacteria with serum albumin, particularly human serum albumin, and/or lysozyme, particularly human lysozyme. In an aspect, methods are provided for treating an antibiotic-resistant Staphylococcus aureus infection in a human comprising the step of contacting the bacteria with or administering to the human a composition comprising an amount of an isolated lysin polypeptide having an SH3-type binding domain effective to kill gram-positive bacteria, further contacting the bacteria with or further administering to the human serum albumin, particularly human serum albumin, and/or lysozyme, particularly human lysozyme. In accordance with the method, serum albumin and the lysin polypeptide may be serially or concomitantly administered, or may be administered in a composition comprising the lysin polypeptide and the serum albumin. In accordance with the method, lysozyme and the lysin polypeptide may be serially or concomitantly administered, or may be administered in a composition comprising the lysin polypeptide and the lysozyme. In an aspect of the methods herein, one or more serum fatty acid is further contacted or administered, particularly selected from one or more of oleate and palmitate.

In accordance with the invention, methods are provided for synergistic killing gram-positive bacteria, particularly Staphylococcus and/or Streptococcus bacteria, comprising contacting the bacteria with a composition comprising an amount of an isolated lysin polypeptide having an SH3-type binding domain effective to kill gram-positive bacteria, wherein the composition further comprises one or more blood component selected from serum albumin and lysozyme, particularly selected from human serum albumin, rabbit serum albumin, dog serum albumin, horse serum albumin, rat serum albumin, and calf (cow/bovine) serum albumin, and human lysozyme. In an aspect, methods are provided for synergistic killing gram-positive bacteria, particularly Staphylococcus and/or Streptococcus bacteria, comprising contacting the bacteria with a composition comprising an amount of an isolated lysin polypeptide having an SH3-type binding domain effective to kill gram-positive bacteria, wherein the composition further comprises one or more blood component selected from serum albumin and lysozyme, particularly selected from human serum albumin, rabbit serum albumin, dog serum albumin, horse serum albumin, and human lysozyme. In an aspect in accordance with the invention, methods are provided for synergistic killing gram-positive bacteria, particularly Staphylococcus and/or Streptococcus bacteria, comprising contacting the bacteria with a composition comprising an amount of an isolated lysin polypeptide having an SH3-type binding domain effective to kill gram-positive bacteria, wherein the composition further comprises one or more blood component selected from serum albumin and lysozyme, particularly human serum albumin and human lysozyme. In an aspect of the invention, methods are provided for synergistic killing of Staphylococcus aureus bacteria comprising the step of contacting the bacteria with a composition comprising an amount of an isolated lysin polypeptide having an SH3-type binding domain effective to kill gram-positive bacteria, wherein the composition further comprises one or more blood component selected from serum albumin and lysozyme, particularly human serum albumin and human lysozyme.

In accordance with the invention, methods are provided for killing gram-positive bacteria comprising the step of contacting the bacteria with a composition comprising an amount of an isolated lysin polypeptide having an SH3-type binding domain effective to kill gram-positive bacteria, wherein the composition further comprises serum albumin and thereafter further contacting the bacteria with lysozyme, particularly human lysozyme. In an aspect of the invention, methods are provided for killing Staphylococcus aureus bacteria resistant to or insensitive to lysozyme comprising the step of contacting the bacteria with a composition comprising an amount of an isolated lysin polypeptide having an SH3-type binding domain effective to kill gram-positive bacteria, wherein the composition further comprises serum albumin and thereafter further contacting the bacteria with lysozyme, particularly human lysozyme.

In an aspect of the invention, methods are provided for killing Staphylococcus aureus bacteria resistant to or insensitive to lysozyme comprising the step of contacting the bacteria with a composition comprising an amount of an isolated lysin polypeptide having an SH3-type binding domain effective to kill gram-positive bacteria, and thereafter further contacting the bacteria with lysozyme, particularly human lysozyme. In an aspect thereof, serum albumin, particularly human serum albumin is additionally administered. In an aspect, the level of serum albumin and/or the binding of native serum albumin to the Staphylococcus aureus bacteria is first assessed or evaluated. If serum albumin is bound to the bacteria then lysozyme is contacted following lysin polypeptide administration, or is administered in combination, including by virtue or a combination composition comprising lysin and lysozyme. If serum albumin is not bound or not adequately bound to the bacteria then serum albumin is first administered, followed by lysin polypeptide administration and then lysozyme administration, or alternatively lysozyme is administered in combination, including by virtue or a combination composition comprising lysin and lysozyme, after serum albumin is first administered. In another aspect, serum albumin is administered concomitantly, sequentially, or in combination with lysin polypeptide, followed by lysozyme administration.

In an aspect of the method the serum albumin may be human, rabbit, dog, horse, rat or calf (cow/bovine) serum albumin. In an aspect of the method the serum albumin may be human, rabbit, dog or horse serum albumin. In an aspect of the method, the serum albumin is human serum albumin, rabbit serum albumin, dog serum albumin, horse serum albumin, rat serum albumin, or calf (cow/bovine) serum albumin, or an active or bacterial binding fragment of peptide thereof particularly an S. aureus binding fragment or peptide of serum albumin. In an aspect of the method, the serum albumin is human serum albumin, rabbit serum albumin, dog serum albumin, or horse serum albumin, or an active or bacterial binding fragment of peptide thereof, particularly an S. aureus binding fragment or peptide of serum albumin. In an aspect of the method, the serum albumin is human serum albumin or an active or bacterial binding fragment of peptide thereof, particularly an S. aureus binding fragment or peptide of serum albumin, particularly of human serum albumin. In an aspect of the method the lysozyme is human lysozyme or an active or lytic fragment or peptide thereof. In an aspect, the lysin polypeptide comprises an SH3 type binding domain. In an aspect, the lytic polypeptide is a chimeric or fusion peptide comprising an SH3-type binding domain capable of binding gram-positive bacteria. In an aspect the lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, and ALE-1 lysin. In an aspect, the lysin polypeptide is PlySs2 (CF-301). In an aspect, the lytic polypeptide is a chimeric or fusion peptide of PlySs2 (CF-301) comprising the PlySs2 (CF-301) SH3-type binding domain capable of binding gram-positive bacteria, particularly Staphylococcus bacteria.

In an aspect, the components, method or assay of the invention are utilized to with regard to lytic polypeptide, particularly lysin comprising an SH3-type binding domain, such as and including PlySs2 (CF-301) polypeptide or a variant or derivative thereof, against gram-positive bacteria. In an aspect, the components, method or assay of the invention are utilized with regard to lytic polypeptide, including PlySs2 (CF-301) polypeptide or a variant or derivative thereof, against antibiotic-resistant bacteria. In an aspect, the components, method or assay of the invention are utilized for lytic polypeptide, including PlySs2 (CF-301) polypeptide or a variant or derivative thereof, against Streptococcus and Staphylococcus bacteria. In an aspect, the components, method or assay of the invention are utilized with regard to lytic polypeptide, including PlySs2 (CF-301) polypeptide or a variant or derivative thereof against antibiotic-resistant Streptococcus and/or Staphylococcus bacteria. In an aspect the lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, and ALE-1 lysin. In an aspect the lytic polypeptide is PlySs2 (CF-301) or a derivative or variant thereof. In an aspect the polypeptide comprises a sequence provided herein.

It has been found that lytic polypeptide, particularly lytic polypeptide having an SH3 binding domain, particularly lytic polypeptide PlySs2 (CF-301), Sal, Lysostaphin, is significantly more active when combined with or in assays comprising added serum albumin and/or lysozyme. Anti-bacterial lytic polypeptide, particularly exemplary lytic polypeptide PlySs2 (CF-301), Sal, lysostaphin, are more active (particularly up to 32 fold to 64 fold to 100 fold more active) in the presence of human serum albumin (as well as serum albumin of or corresponding to that of other species, particularly horse, dog and rabbit), and/or in the presence of lysozyme, particularly human lysozyme, than in broth, such as cation-adjusted broth, without serum albumin added.

In an aspect of the present invention, bacteriophage lysin derived from Streptococcus or Staphylococcus bacteria and/or effective against Streptococcus and/or Staphylococcus bacteria are utilized in the methods, assays, compositions, formulations, and/or constructs of the invention. In a particular aspect, the lysin comprises an SH3-type bacterial binding domain. Exemplary lysin polypeptide(s) of use or applicable in the present invention, including PlySs2 (CF-301) lysin, Sal lysin, LysK lysin, lysostaphin, phill lysin, LysH5 lysin, MV-L lysin, LysGH15 lysin, and ALE-1 lysin, particularly PlySs2 (CF-301) lysin are provided herein. In one such aspect, the lysin is capable of killing Staphylococcus aureus strains and bacteria, as demonstrated herein. In an aspect, the lysin is capable of killing Staphylococcus and Streptococcus bacteria. In an aspect, the lysin is effective against antibiotic-resistant Staphylococcus aureus such as methicillin-resistant Staphylococcus aureus (MRSA), vancomycin resistant Staphylococcus aureus (VRSA), daptomycin-resistant Staphylococcus aureus (DRSA) and linezolid-resistant Staphylococcus aureus (LRSA). The lysin may be effective against vancomycin intermediate-sensitivity Staphylococcus aureus (VISA).

In one such aspect, the lysin is PlySs2 (CF-301) lysin and is capable of killing Staphylococcus aureus strains and bacteria, as demonstrated herein. In an aspect, the PlySs2 (CF-301) lysin is capable of killing Staphylococcus and Streptococcus bacteria. PlySs2 (CF-301) is effective against antibiotic-resistant Staphylococcus aureus such as methicillin-resistant Staphylococcus aureus (MRSA), vancomycin resistant Staphylococcus aureus (VRSA), daptomycin-resistant Staphylococcus aureus (DRSA) and linezolid-resistant Staphylococcus aureus (LRSA). PlySs2 (CF-301) is effective against vancomycin intermediate-sensitivity Staphylococcus aureus (VISA).

The isolated lysin polypeptide may comprise the lysin amino acid sequence provided herein or variants thereof having at least 80% identity, 85% identity, 90% identity, 95% identity or 99% identity to the polypeptide herein and effective to kill the gram-positive bacteria. The isolated PlySs2 (CF-301) lysin polypeptide may comprise the PlySs2 (CF-301) amino acid sequence provided herein (SEQ ID NO:3) or variants thereof having at least 80% identity, 85% identity, 90% identity, 95% identity or 99% identity to the polypeptide herein (SEQ ID NO:3) and effective to kill Staphylococcus and Streptococcus bacteria. The isolated Sal lysin polypeptide may comprise the Sal1 amino acid sequence provided herein (SEQ ID NO:5) or variants thereof having at least 80% identity, 85% identity, 90% identity, 95% identity or 99% identity to the polypeptide herein (SEQ ID NO:5) and effective to kill Staphylococcus bacteria. The isolated LysK lysin polypeptide may comprise the LysK amino acid sequence provided herein (SEQ ID NO:6) or variants thereof having at least 80% identity, 85% identity, 90% identity, 95% identity or 99% identity to the polypeptide herein (SEQ ID NO:6) and effective to kill Staphylococcus bacteria. The isolated lysostaphin lysin polypeptide may comprise the lysostaphin amino acid sequence provided herein (SEQ ID NO:7) or variants thereof having at least 80% identity, 85% identity, 90% identity, 95% identity or 99% identity to the polypeptide herein (SEQ ID NO:7) and effective to kill Staphylococcus bacteria, or Staphylococcus and Streptococcus bacteria. The isolated lysin polypeptide may comprise the lysin polypeptide amino acid sequence provided herein or known in the art, including as by reference herein, variants thereof having at least 80% identity, 85% identity, 90% identity, 95% identity or 99% identity to the polypeptide herein or known or recognized in the art and effective to kill Staphylococcus bacteria, or Staphylococcus and Streptococcus bacteria.

In any such above method or methods, the bacteria may be selected from Staphylococcus aureus, Listeria monocytogenes, Staphylococcus simulans, Streptococcus suis, Staphylococcus epidermidis, Streptococcus equi, Streptococcus equi zoo, Streptococcus agalactiae (GBS), Streptococcus pyogenes (GAS), Streptococcus sanguinis, Streptococcus gordonii, Streptococcus dysgalactiae, Group G Streptococcus, Group E Streptococcus, Enterococcus faecalis and Streptococcus pneumonia.

In accordance with any of the methods of the invention, bacteria may be an antibiotic resistant bacteria. The bacteria may be methicillin-resistant Staphylococcus aureus (MRSA), vancomycin intermediate-sensitivity Staphylococcus aureus (VISA), vancomycin resistant Staphylococcus aureus (VRSA), daptomycin-resistant Staphylococcus aureus (DRSA), or linezolid-resistant Staphylococcus aureus (LRSA). The susceptible bacteria may be a clinically relevant or pathogenic bacteria, particularly for humans. In an aspect of the method(s), the lysin polypeptide(s) is effective to kill Staphylococcus, Streptococcus, Enterococcus and Listeria bacterial strains.

In an additional aspect or embodiment of the methods and compositions provided herein, another distinct staphylococcal specific lysin is used herein alone or in combination with lysin provided herein, including the PlySs2 (CF-301) lysin as provided and described herein. In one such aspect or embodiment of the methods and compositions provided herein, one or more lysin selected from Sal lysin, LysK lysin, lysostaphin, phill lysin, LysH5 lysin, MV-L lysin, LysGH15 lysin, and ALE-1 lysin is used herein alone or in combination with the PlySs2 (CF-301) lysin as provided and described herein. In an aspect or embodiment of the methods and compositions provided herein, one or more lysin selected from Sal lysin, LysK lysin, lysostaphin, phill lysin, LysH5 lysin, MV-L lysin, LysGH15 lysin, and ALE-1 lysin are in combination with one another as provided and described herein. In an aspect or embodiment of the methods and compositions provided herein, one or more SH3 binding domain of lysin selected from Sal lysin, LysK lysin, lysostaphin, phill lysin, LysH5 lysin, MV-L lysin, LysGH15 lysin, and ALE-1 lysin are used in combination with at least one other catalytic domain of another lysin, including as provided and described herein.

Other objects and advantages will become apparent to those skilled in the art from a review of the following description which proceeds with reference to the following illustrative drawings.

### BRIEF DESCRIPTION OF THE DRAWINGS

FIG. 1A-1D depict enhancement of PlySs2 (CF-301) bacteriolytic activity in human blood matrices. Composite time kill curves of PlySs2 (CF-301) are compared to buffer controls against S. aureus strain MW2 tested in (A) human serum from 16 individuals and 4 pooled samples, (B) whole human blood of 10 individuals, (C) MHB (10 replicates), and (D) pooled BALB/c mouse serum. Mean values (±SEM) are shown for each time-point.

FIG. 2A-2N provide a survey of the human blood effect on a range of S. aureus strains as indicated. Composite time kill curves for PlySs2 (CF-301) are compared to buffer controls against the indicated S. aureus strains tested using 5 replicates in either MHB or pooled human serum. Mean values (±SEM) are shown for each time-point.

FIG. 3A-3D depict the human blood effect on another lysin-like protein (lysostaphin) and a small molecule antibiotic (vancomycin). Composite time kill curves for the indicated compounds are compared to buffer controls against S. aureus strain MW2. A and B, Lysostaphin tested using 5 replicate samples in either MHB or pooled human serum, respectively. C and D, Vancomycin tested using 5 replicate samples in either MHB or pooled human serum, respectively. Mean values (±SEM) are shown for each time-point.

FIGS. 4A-4D depict enhancement of PlySs2 (CF-301) bacteriolytic activity in animal blood matrices. Composite time kill curves of PlySs2 (CF-301) are compared to buffer controls against S. aureus strain MW2 tested in (A) fetal calf serum (3 replicates each of 3 different lots), (B) Sprague Dawley rat serum blood (5 replicates each of two pooled lots), (C) rabbit serum (5 replicates each of two pooled mixed species lots), and (D) Beagle dog serum from 8 individual animals. Mean values (±SEM) are shown for each time-point.

FIG. 5 provides PlySs2 (CF-301) MIC distribution for S. aureus MW2 in different human blood matrices. Histograms are shown for analyses performed in multiple different samples, including individual and pooled, of whole blood (n=13), serum (n=32), and plasma (n=15). Each sample is described in Supplementary Table 1. The MICs are plotted on the x axis, and the numbers of patient samples with particular MICs are plotted on the y axis.

FIG. 6A-6D demonstrates that PlySs2 (CF-301) exhibits potent synergy with other antimicrobial agents in human serum. Composite time kill curves for the indicated single agents (concentrations in parentheses) are compared to both buffer controls and the combination of both agents (each at indicated single agent concentrations) against S. aureus strain MW2. Mean values (±SEM) are shown for each time-point based on assays performed in triplicate. A-C, PlySs2 (CF-301) tested at sub-MIC amounts in the presence of a constant amount of DAP. D, PlySs2 (CF-301) and lysostaphin tested using sub-MIC amounts.

FIGS. 7A-7C provide colorimetric MIC determination of against S. aureus MW2 using Alamar Blue® at 18 hours. The addition of Alamar blue dye (resazurin) to each well for 2 hours enables an assessment of viability (pink) and cell death (blue) over a log 10 dilution range. A, Viability assay in MHB, HuS and MuS. Samples were assayed in HuS that was either untreated or pretreated for 3 hours with proteinase K-agarose beads. As a control for protease carry-over, the proteinase K-pretreated serum was diluted 3:4 into untreated serum prior to analysis. B, Viability assay in HuS pretreated for 30 minutes over a range of temperatures. C, Checkerboard analysis of PlySs2 (CF-301) in combination with HuS with MHB as the diluent. Red squares indicate HuS dilutions at which the enhancer effect is diminished.

FIG. 8A-8C depicts the effect of HuLYZ and HSA on PlySs2 (CF-301) activity. A provides a time-kill assay combining a sub-MIC amount of lysin with a range of huLYZ concentrations. B and C depict a second in vitro assay based on loss of optical density in a treated culture, with addition of HuLYZ (B) and HSA (C).

FIGS. 9A-9C provide Western blot studies using anti-PlySs2 (CF-301) antibody.

FIG. 10 depicts labeling of PlySs2 (CF-301) (red) in the presence and absence of rHSA. In data not shown, neither of PlyG^{GFP} or PlyC^{AF} at 25 μg/ml label with or without HSA.

FIG. 11 depicts the effect of preincubation of different serum types (and MHB) on subsequent labeling of PlySs2 (CF-301) (red).

FIG. 12 depicts labeling with HuLYZ (green) in the presence and absence of PlySs2 (CF-301) (1×-0.25×MIC).

FIG. 13 depicts TEM analysis of S. aureus strain MW2 treated with PlySs2 (CF-301) in either human serum (HuS) or MHB for 15 minutes.

FIGS. 14A and 14B depicts efficacy in the rat (A) and rabbit (B) infective endocarditis (IE) model for various PlySs2 (CF-301) dosing regimens added to daptomycin. Data are plotted as treatment regimen vs average log₁₀ CFU/g tissue for each dose group. Medians±SEM are also shown.

FIG. 15 presents AUC values and AUC dose proportionality for various PlySs2 (CF-301) dosing regimens in rats and rabbits.

FIG. 16 shows the amino acid sequence of PlySs2 (CF-301) (SEQ ID NO:3), as well as a schematic illustration of the PlySs2 (CF-301) polypeptide lysin's N-terminal CHAP domain and C-terminal SH3-type binding domain connected to each other via a linker.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes 1-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

In addition, various terms are utilized and made reference to in accordance with the invention, and may have the definitions or descriptions as provided herein and below.

In a general aspect of the present invention, it has been recognized that certain lysins, particularly lysins having an SH-3 type binding domain, including PlySs2 (CF-301) lysin, are more effective in human blood, serum and plasma than in artificial media. An increase and enhanced activity up to 100 fold has been identified. In addition to human serum, the enhancer effect is also observed in the serum of rabbits, dogs and horses. An intermediary effect is observed in rat serum. Mouse serum does not demonstrate the enhancer effect. The inventors hypothesize and now demonstrate that certain phage lysins, particularly lysins having an SH-3 type binding domain, including PlySs2 (CF-301) are capable of favorable antibacterial interactions with one or more components in blood or blood fractions.

In accordance with the invention, blood components have been identified that synergize with lysin, including PlySs2 (CF-301). In one aspect, serum albumin, particularly human serum albumin, enhances the antibacterial activity of lysin, including PlySs2 (CF-301) lysin. Serum albumin is the most abundant protein in human blood plasma, constituting about half of serum protein. The reference range for albumin concentration in serum is approximately 35-50 g/L or 3.5-5.0 g/dL. Albumin has a serum half-life of approximately 20 days.

The gene for albumin is located on chromosome 4 and is split into 15 exons that are symmetrically placed within 3 domains thought to have arisen by triplication of a single primordial domain. Albumin transports hormones, fatty acids, and other compounds, buffers pH, and maintains oncotic pressure, among other functions. The amino acid sequence of human serum albumin (Uniprot P02768) is as follows (SEQ ID NO:1):

(SEQ ID NO: 1)
MKWVTFISLLFLFSSAYSRGVFRRDAHKSEVAHREKDLGEENFKALVLI
AFAQYLQQCPFEDHVKLVNEVTEFAKTCVADESAENCDKSLHTLPGDKL
CTVATLRETYGENADCCAKQEPERNECFLQHKDDNPNLPRLVRPEVDVM
CTAFHDNEETFLKKYLYEIARRHPYFYAPELLFFAKRYKAAFTECCQAA
DKAACLLPKLDELRDEGKASSAKQRLKCASLQKFGERAFKAWAVARLSQ
RFPKAEFAEVSKLVTDLTKVHTECCHGDLLECADDRADLAKYICENQDS
ISSKLKECCEKPLLEKSHCIAEVENDEMPADLPSLAADEVESKDVCKNY
AEAKDVELGMFLYEYARRHPDYSVVLLLRLAKTYETTLEKCCAAADPHE
CYAKVEDEFKPLVEEPQNLIKQNCELFEQLGEYKFQNALLVRYTKKVPQ
VSTPTLVEVSRNLGKVGSKCCKHPEAKRNPCAEDYLSVVINQLCVLHEK
TPVSDRVTKCCTESLVNRRPCFSALEVDETYVPKEFNAETFTFHADICT
LSEKERQIKKQTALVELVKHKPKATKEQLKAVMDDFAAFVEKCCKADDK
ETCFAEEGKKLVAASQAALGL

Of the 609 amino acids in this sequence, 585 amino acids are observed in the final product present in the blood; the first 24 amino acids (here italicized and underlined), including the signal peptide (1-18) and propeptide portions, are cleaved after translation.

In a further aspect, lysozyme, particularly human lysozyme, enhances the antibacterial activity of lysin, including PlySs2 (CF-301) lysin. Lysozyme, also known as muramidase or N-acetylmuramide glycanhydrolase is an antimicrobial enzyme produced by animals that forms part of the innate immune system. Lysozyme catalyzes the hydrolysis of 1,4-beta-linkages between N-acetylmuramic acid (NAM) and N-acetyl-D-glucosamine (NAG) residues in peptidoglycan, the major component of gram-positive bacterial cell wall, in turn compromising the integrity of bacterial cell walls causing lysis of the bacteria. Notably, Staphylococcus aureus bacteria are completely lysozyme resistant, which greatly contributes to their persistence and success in colonizing the skin and mucosal areas of humans and animals (Bera, A et al (2004) Molecular Microbiology 55(3):778-787; Pushkaran, A C et al (2015) J Chem Inf Model 55(4):760-770).

Human lysozyme is a protein of 148 amino acids, including a signal peptide sequence of 18 amino acids, providing a mature 129 amino acid protein. The sequence of human lysozyme corresponds to Uniprot P61626 and Genbank NP_000230 and is as follows (SEQ ID NO:2) (the signal peptide is italicized and underlined):

(SEQ ID NO: 2)
MKALIVLGLVLLSVTVQGKVFERCELARTLKRLGMDGYRGISLANWMCL
AKWESGYNTRATNYNAGDRSTDYGIFQINSRYWCNDGKTPGAVNACHLS
CSALLQDNIADAVACAKRVVRDPQGIRAWVAWRNRCQNRDVRQYVQGCG
V

In accordance with the present invention a lysin polypeptide of use and relevance in the invention may particularly be a lysin polypeptide having an SH3-type binding domain. A Src homology 3 (SH3) enzyme domain has a characteristic beta-barrel fold that consists of five or six β-strands arranged as two tightly packed anti-parallel β sheets. The classical SH3 domain is usually found in proteins that interact with other proteins and mediate assembly of specific protein complexes, including via binding to proline-rich peptides in their respective binding partner. Many SH3-binding epitopes of proteins have a Proline containing sequence motif. SH3 domains and sequences are described and reviewed in the prior art including in Whisstock, J. C. and Lesk, A. M. (1999) TIBS 24:132-133 and Ponting, C. P. et al (1999) J Mol Biol 289:729-745.

In an aspect thereof a lysin polypeptide having an SH3-type binding domain is selected from PlySs2 (CF-301) lysin, Sal lysin, LysK lysin, lysostaphin, phill lysin, LysH5 lysin, MV-L lysin, LysGH15 lysin, and ALE-1 lysin. In an aspect, the lysin polypeptide is PlySs2.

The terms “PlySs lysin(s)”, “PlySs2 lysin”, “PlySs2”, “PlySs2 (CF-301)”, “PlySs2/CF-301”, “CF-301”, “CF301” and any variants not specifically listed, may be used herein interchangeably, and as used throughout the present application and claims refer to proteinaceous material including single or multiple proteins, and extends to those proteins having the amino acid sequence data described herein and presented below, and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “PlySs lysin(s)”, “PlySs2 lysin”, “PlySs2”, “PlySs2 (CF-301)”, “PlySs2/CF-301”, “CF-301”, “CF301” are intended to include within their scope proteins specifically recited herein as well as all substantially homologous analogs, fragments or truncations, and allelic variations. PlySs2 lysin is described in U.S. Pat. No. 9,034,322 and PCT Application PCT/US2012/34456. Gilmer et al also describes PlySs2 lysin (Gilmer D B et al (2013) Antimicrob Agents Chemother Epub 2013 Apr. 9 [PMID 23571534]). The PlySs2 (CF-301) amino acid sequence is provided below (SEQ ID NO:3). The PlySs2 (CF-301) polypeptide lysin N-terminal CHAP domain (cysteine-histidine amidohydrolase/peptidase) (starting with LNNV . . . and ending with . . . HYIT) and C-terminal SH3-type binding domain (starting with RSYR . . . and ending with . . . YVAT) are underlined below.

| | |
|---|---|
| MTTVNEALNN VRAOVGSGVS VGNGECYALA SWYERMISPD ATVGLGAGVG WVSGAIGDTI | 60 |
| SAKNIGSSYN WQANGWTVST SGPFKAGQIV TLGATPGNPY GHVVIVEAVD GDRLTILEQN | 120 |
| YGGKRYPVRN YYSAASYRQQ VVHYITPPGT VAQSAPNLAG SRSYRETGTM TVTVDALNVR | 180 |
| RAPNTSGEIV AVYKRGESFD YDTVIIDVNG YVWVSYIGGS GERNYVATGA TKDGKRFGNA | 240 |
| WGTFK | 245 |

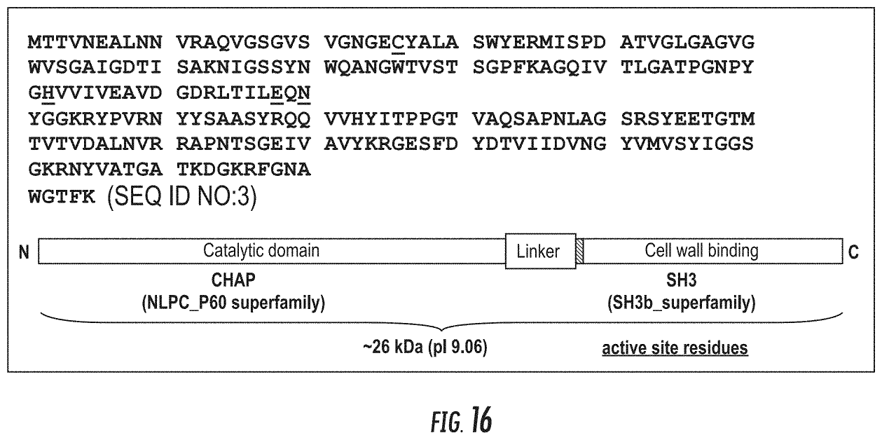

(SEQ ID NO:3). A schematic of the PlySs2 (CF-301) polypeptide lysin N-terminal CHAP domain and C-terminal SH3-type binding domain are provided in FIG. 16, showing the SH3-type binding domain connected to the CHAP domain via a linker.

The term “ClyS”, “ClyS lysin” refers to a chimeric lysin ClyS, with activity against Staphylococci bacteria, including Staphylococcus aureus, is detailed in WO 2010/002959 and also described in Daniel et al (Daniel, A et al (2010) Antimicrobial Agents and Chemother 54(4):1603-1612). ClyS does not have an SH3-type binding domain. Exemplary amino acid sequence of ClyS is provided below (SEQ ID NO:4).

| (SEQ ID NO: 4) | |
|---|---|
| METLKQAESY IKSKVNTGTD FDGLYGYQCM DLAVDYIYHV TDGKIRMWGN AKDAINNSFG | 60 |
| GTATVYKNYP AFRPKYGDVV VWTTGNFATY GHIAIVTNPD PYGDLQYVTV LEQNWNGNGI | 120 |
| YKTELATIRT HDYTGITHFI RPNFATESSV KKKDTKKKPK PSNRDGLNKD KIVYDRTNIN | 180 |
| YNMVLQGKSA SKITVGSKAP YNLKWSKGAY FNAKIDGLGA TSATRYGDNR TNYREDVGQA | 240 |
| VYAPGTLIYV FEIIDGWCRI YWNNHNEWIW HERLIVKEVF. | 280 |

Sal lysin, alternatively denoted Sal1, is described in several Yoon et al references including U.S. Pat. Nos. 8,232,370, 8,377,431 and 8,377,866. Exemplary Sal 1 sequence is provided below (SEQ ID NO:5).

| (SEQ ID NO: 5) | |
|---|---|
| 1 | MAKTQAEINK RLDAYAKGTV DSPYRIKKAT SYDPSFGVME AGAIDADGYY RAQCQDLITD |
| 61 | YVLWLTDNKV RTWGNAKDQI KQSYGTGFKI HENKPSTVPK KGWIAVFTSG SYQQWGHIGI |
| 121 | VYDGGNTSTF TILEQNWNGY ANKKPTKRVD NYYGLTHFIE IPVKAGTTVK KETAKKSASK |
| 181 | TPAPKYEATL KVSKNHINYT MDKRGKKPEG MVIHNDAGRS SGQQYENSLA NAGYARYANG |
| 214 | IAHYYGSEGY VWEAIDAKNQ IAWHTGDGTG ANSGNFRFAG IEVCQSMSAS DAOFLKNEQA |
| 301 | VFQFTAEKEK FWGITPNRKT VRLHMEFVPT ACPHRSMVIH TGFNPVTQGR PSQAIMNKIK |
| 361 | DYFIKQIKNY MDKGTSSSTV VKDGKTSSAS TPATRPVTGS WKKNQYGTWY KPENATFVNG |
| 421 | NQPIVTRIGS PFLNAPVGGN LPAGATIVYD EVCIQAGHIW IGYNAYNGNR VYCPVRTCQG |
| 481 | VPPNHIPGVA WGVFK* |

LysK lysin is an anti-staphylococcal lysin and includes an amidase, CHAP domain and an SH3-type binding domain. LysK is described in O'Flaherty S et al (2005) J Bacteriol 187(20):7161-7164. Fusion proteins, particularly LysK and lysostaphin fusions are described in Donovan et al U.S. Pat. No. 8,568,714. Chimerics/chimeric lysins having Ply187 endopeptidase domain and LysK SH3 cell wall binding domain are described in U.S. Ser. No. 13/432,758 and WO2013/149010. The LysK sequence is provided below (SEQ ID NO:6).

| (SEQ ID NO: 6) | |
|---|---|
| 1 | MAKTQAEINK RLDAYAKGTV DSPYRVKKAT SYDPSFGVME AGAIDADGYY HAQCQDLITD |
| 61 | YVLWLTDNKV RTWGNAKDQI KQSYGTGFKI HENKPSTVPK KGWIAVFTSG SYEQWGEIGI |
| 121 | VYDGGNTSTF TILEQNWNGY ANKKPTKRVD NYYGLTHFIE IPVKAGTTVK KKTAKKSASK |
| 181 | TPAPKYEATL KVSKNHINYT MDKRGKKPEG MVIENDAGRS SGQQWENSLA NAGYARYANG |
| 241 | IAHYYGSEGY VWEAIDAKNQ IAWETGDGTG ANSGNERFAG IEVCQSMSAS DAQFLKNEQA |
| 301 | VFQFTAEKEK EWGLTPNRKT VRLHMEFVPT ACPHRSMVLH TGENPVTQGR PSQAIMNKLK |
| 361 | DYFIKQIKNY MDKGTSSSTV VKDGKTSSAS TPATRPVTGS WKKNQYGTWY KPENATFVNG |
| 421 | NQPIVTRIGS PFLNAPVGGN LPAGATIVYD EVCIQAGHIW IGYNAYNGNR VYCPVRTCQG |
| 481 | VPPNQIPGVA WGVFK |

It is notable that Sal-1 and LysK are very similar in sequence and differ only at amino acids 26, 114 and 485, underlined in each sequence above.

Lysostaphin and recombinant lysostaphin are described in various references including Sloan G L et al (1982) Int J Sys Bacteriol 32:170-174 and Oldham E R and Daley M J (1991) J Dairy Science 74:1127-1131. Cloned lysostaphin sequence from Staphylococcus simulans is described by Recsei P A et al (1987) PNAS USA 84:1127-1131 and is provided in U.S. Pat. No. 4,931,390. Mature lysostaphin consists of 246 amino acid residues. The preprotein comprises three distinct regions in the precursor protein: a typical signal peptide (about 30-38 aa), a hydrophilic and highly ordered protein domain with 14 repetitive sequences (296 aa) and the hydrophobic mature lysostaphin. Exemplary sequence of lysostaphin is provided below (SEQ ID NO:7). Mature sequence is in bold.

(SEQ ID NO: 7)
>sp|P10547|LSTP_STASI Lysostaphin
OS = Staphylococcus simulans
MKKTKNNYYTRPLAIGLSTFALASIVYGGIQNETHASEKSNMDVSKKVA
EVETSKAPVENTAEVETSKAPVENTAEVETSKAPVENTAEVETSKAPVE
NTAEVETSKAPVENTAEVETSKAPVENTAEVETSKAPVENTAEVETSKA
PVENTAEVETSKAPVENTAEVETSKAPVENTAEVETSKAPVENTAEVET
SKAPVENTAEVETSKAPVENTAEVETSKAPVENTAEVETSKALVORTAL
RAATHENSAWLNWYKKGYGYGPYPLGINGGMHYGVDFFMNIGTPVKAIS
SGKIVEAGWSNYGGGNQIGLIENDGVHRQWYMHLSKYNVKVGDYVKAGQ
IIGWSGSTGYSTAPHLHEVRMVNSFSNSTAWPMPFLKSAGYGKAGGTVT
PTPNTGWKTNKYGTLYKSESASFTPNTDIITRTTGPFRSMPQSGVLKAG
QTIHYDEVMKQDGHVWVGYTGNSGQRIYLPVRTWNKSTNTLGVLWGTIK.

ALE-1 lysin is a homologue of lysostaphin and described in Liu J Z et al (2006) J Biol Chem 281:549-558.

Phill bacteriophage lysin comprises two hydrolase domains, an endopeptidase and amidase, and an SH3B type binding domain and is known and described in the art, including Donovan D M et al (2006) FEMS Microbiol Lett 265(1):133-139. LysH5 lysin is similarly comprised of three domains, two hydrolase domains, an endopeptidase and amidase, and an SH3B type binding domain, and is described and known including in Obeso J M et al (2008) Int J Food Microbiol 128(2):212-228. Phage lysin MV-L is described in Rashel M et al (2007) J Infect Dis 196(8):1237-1247. MV-L lysin is comprised of two catalytic domains (an endopeptidase and an amidase domain) linked to a single cell wall targeting (CWT) domain, a type of binding domain. Unless otherwise indicated, references herein to a “binding domain” herein include a CWT domain. The MV-L CWT domain, like the staphylolytic enzyme lysostaphin, displays homology to SH3b-like domains. The SH3b-like domains bind to the peptide cross-bridge (the penta Glycine) in the staphylococcal cell wall.

### Polypeptides and Lytic Enzymes

A “lytic enzyme” includes any bacterial cell wall lytic enzyme that kills one or more bacteria under suitable conditions and during a relevant time period. Examples of lytic enzymes include, without limitation, various amidase cell wall lytic enzymes.

A “S. suis lytic enzyme” includes a lytic enzyme that is capable of killing at least one or more Streptococcus suis bacteria under suitable conditions and during a relevant time period.

A “bacteriophage lytic enzyme” refers to a lytic enzyme extracted or isolated from a bacteriophage or a synthesized lytic enzyme with a similar protein structure that maintains a lytic enzyme functionality.

A lytic enzyme is capable of specifically cleaving bonds that are present in the peptidoglycan of bacterial cells to disrupt the bacterial cell wall. It is also currently postulated that the bacterial cell wall peptidoglycan is highly conserved among most bacteria, and cleavage of only a few bonds to may disrupt the bacterial cell wall. The bacteriophage lytic enzyme may be an amidase, although other types of enzymes are possible. Examples of lytic enzymes that cleave these bonds are various amidases such as muramidases, glucosaminidases, endopeptidases, or N-acetyl-muramoyl-L-alanine amidases. Fischetti et al (1974) reported that the C1 streptococcal phage lysin enzyme was an amidase. Garcia et al (1987, 1990) reported that the Cp1 lysin from a S. pneumoniae from a Cp-1 phage was a lysozyme. Caldentey and Bamford (1992) reported that a lytic enzyme from the phi 6 Pseudomonas phage was an endopeptidase, splitting the peptide bridge formed by melo-diaminopimilic acid and D-alanine. The E. coli T1 and T6 phage lytic enzymes are amidases as is the lytic enzyme from Listeria phage (ply) (Loessner et al, 1996). There are also other lytic enzymes known in the art that are capable of cleaving a bacterial cell wall.

A “lytic enzyme genetically coded for by a bacteriophage” includes a polypeptide capable of killing a host bacteria, for instance by having at least some cell wall lytic activity against the host bacteria. The polypeptide may have a sequence that encompasses native sequence lytic enzyme and variants thereof. The polypeptide may be isolated from a variety of sources, such as from a bacteriophage (“phage”), or prepared by recombinant or synthetic methods, such as those described by Garcia et al and also as provided herein. The polypeptide may comprise a choline-binding portion at the carboxyl terminal side and may be characterized by an enzyme activity capable of cleaving cell wall peptidoglycan (such as amidase activity to act on amide bonds in the peptidoglycan) at the amino terminal side. Lytic enzymes have been described which include multiple enzyme activities, for example two enzymatic domains, such as PlyGBS lysin. Generally speaking, a lytic enzyme may be between 25,000 and 35,000 daltons in molecular weight and comprise a single polypeptide chain; however, this can vary depending on the enzyme chain. The molecular weight most conveniently can be determined by assay on denaturing sodium dodecyl sulfate gel electrophoresis and comparison with molecular weight markers.

“A native sequence phage associated lytic enzyme” includes a polypeptide having the same amino acid sequence as an enzyme derived from a bacteria. Such native sequence enzyme can be isolated or can be produced by recombinant or synthetic means.

The term “native sequence enzyme” encompasses naturally occurring forms (e.g., alternatively spliced or altered forms) and naturally-occurring variants of the enzyme. In one embodiment of the invention, the native sequence enzyme is a mature or full-length polypeptide that is genetically coded for by a gene from a bacteriophage specific for Streptococcus or capable of killing Streptococcus. Of course, a number of variants are possible and known, as acknowledged in publications such as Lopez et al., Microbial Drug Resistance 3: 199-211 (1997); Garcia et al., Gene 86: 81-88 (1990); Garcia et al., Proc. Natl. Acad. Sci. USA 85: 914-918 (1988); Garcia et al., Proc. Natl. Acad. Sci. USA 85: 914-918 (1988); Garcia et al., Streptococcal Genetics (J. J. Ferretti and Curtis eds., 1987); Lopez et al., FEMS Microbiol. Lett. 100: 439-448 (1992); Romero et al., J. Bacteriol. 172: 5064-5070 (1990); Ronda et al., Eur. J. Biochem. 164: 621-624 (1987) and Sanchez et al., Gene 61: 13-19 (1987). The contents of each of these references, particularly the sequence listings and associated text that compares the sequences, including statements about sequence homologies, are specifically incorporated by reference in their entireties.

“A variant sequence lytic enzyme” includes a lytic enzyme characterized by a polypeptide sequence that is different from that of a naturally occurring lytic enzyme, but retains functional activity. The lytic enzyme can, in some embodiments, be genetically coded for by a bacteriophage specific for bacteria such as Streptococcus having a particular amino acid sequence identity with the lytic enzyme sequence(s) hereof, as provided or referenced herein. For example, in some embodiments, a functionally active lytic enzyme can kill Streptococcus bacteria, and other susceptible bacteria as provided herein, including by disrupting the cellular wall of the bacteria. An active lytic enzyme may have a 60, 65, 70, 75, 80, 85, 90, 95, 97, 98, 99 or 99.5% amino acid sequence identity with the lytic enzyme sequence(s) hereof, as provided or referenced herein. Such phage associated lytic enzyme variants include, for instance, lytic enzyme polypeptides wherein one or more amino acid residues are added, or deleted at the N or C terminus of the sequence of the lytic enzyme sequence(s) hereof, as provided or referenced herein. In a particular aspect, a phage associated lytic enzyme will have at least about 80% or 85% amino acid sequence identity with native phage associated lytic enzyme sequences, particularly at least about 90% (e.g. 90%) amino acid sequence identity. Most particularly a phage associated lytic enzyme variant will have at least about 95% (e.g. 95%) amino acid sequence identity with the native phage associated the lytic enzyme sequence(s) hereof, as provided or referenced herein.

“Percent amino acid sequence identity” with respect to the phage associated lytic enzyme sequences identified is defined herein as the percentage of amino acid residues in a candidate sequence that are identical with the amino acid residues in the phage associated lytic enzyme sequence, after aligning the sequences in the same reading frame and introducing gaps, if necessary, to achieve the maximum percent sequence identity, and not considering any conservative substitutions as part of the sequence identity.

“Percent nucleic acid sequence identity” with respect to the phage associated lytic enzyme sequences identified herein is defined as the percentage of nucleotides in a candidate sequence that are identical with the nucleotides in the phage associated lytic enzyme sequence, after aligning the sequences and introducing gaps, if necessary, to achieve the maximum percent sequence identity.

To determine the percent identity of two nucleotide or amino acid sequences, the sequences are aligned for optimal comparison purposes (e.g., gaps may be introduced in the sequence of a first nucleotide sequence). The nucleotides or amino acids at corresponding nucleotide or amino acid positions are then compared. When a position in the first sequence is occupied by the same nucleotide or amino acid as the corresponding position in the second sequence, then the molecules are identical at that position. The percent identity between the two sequences is a function of the number of identical positions shared by the sequences (i.e., % identity=# of identical positions/total # of positions.times.100).

The determination of percent identity between two sequences may be accomplished using a mathematical algorithm. A preferred, non-limiting example of a mathematical algorithm utilized for the comparison of two sequences is the algorithm of Karlin et al., Proc. Natl. Acad. Sci. USA, 90:5873-5877 (1993). Such an algorithm is incorporated into the NBLAST program which may be used to identify sequences having the desired identity to nucleotide sequences of the invention. To obtain gapped alignments for comparison purposes, Gapped BLAST may be utilized as described in Altschul et al., Nucleic Acids Res, 25:3389-3402 (1997). When utilizing BLAST and Gapped BLAST programs, the default parameters of the respective programs (e.g., NBLAST) may be used. See the programs provided by National Center for Biotechnology Information, National Library of Medicine, National Institutes of Health. In one embodiment, parameters for sequence comparison may be set at W=12. Parameters may also be varied (e.g., W=5 or W=20). The value “W” determines how many continuous nucleotides must be identical for the program to identify two sequences as containing regions of identity.

“Polypeptide” includes a polymer molecule comprised of multiple amino acids joined in a linear manner. A polypeptide can, in some embodiments, correspond to molecules encoded by a polynucleotide sequence which is naturally occurring. The polypeptide may include conservative substitutions where the naturally occurring amino acid is replaced by one having similar properties, where such conservative substitutions do not alter the function of the polypeptide (see, for example, Lewin “Genes V” Oxford University Press Chapter 1, pp. 9-13 1994).

The term “altered lytic enzymes” includes shuffled and/or chimeric lytic enzymes.

Phage lytic enzymes specific for bacteria infected with a specific phage have been found to effectively and efficiently break down the cell wall of the bacterium in question. The lytic enzyme is believed to lack proteolytic enzymatic activity and is therefore non-destructive to mammalian proteins and tissues when present during the digestion of the bacterial cell wall. As shown by Loeffler et al., “Rapid Killing of Streptococcus pneumoniae with a Bacteriophage Cell Wall Hydrolase,” Science, 294: 2170-2172 (Dec. 7, 2001), and supplemental material thereto published online by Science magazine, which are incorporated herein by reference in their entirety, a purified pneumococcal bacteriophage lytic enzyme, such as Pal, is able to kill various pneumococci. Loeffler et al. have shown through these experiments that within seconds after contact, the lytic enzyme Pal is able to kill 15 clinical stains of S. pneumoniae, including the most frequently isolated serogroups and penicillin resistant strains, in vitro. Treatment of mice with Pal was also able to eliminate or significantly reduce nasal carriage of serotype 14 in a dose-dependent manner. Furthermore, because it has been found that the action of Pal, like other phage lytic enzymes, but unlike antibiotics, was rather specific for the target pathogen, it is likely that the normal flora will remain essentially intact (M. J. Loessner, G. Wendlinger, S. Scherer, Mol Microbiol 16, 1231-41. (1995) incorporated herein by reference). In contrast, certain lysin polypeptides of the present invention have remarkably broad and clinically significant bacterial killing profile. For example, the isolated S. suis lysin PlySs2, is effective in killing S. suis, and also various other Streptococcus strains, including Group B Streptococcus (GBS), Staphylococcal strains, including Staphylococcus aureus, Enterococcus and Listeria. The lysin of the present invention may demonstrates a breadth of bacterial cell killing across Staphylococcus and/or Streptococcus strains or bacteria.

The lytic enzyme(s) or polypeptide(s) may be truncated, chimeric, shuffled or “natural,” and may be in combination. Relevant U.S. Pat. No. 5,604,109 is incorporated herein in its entirety by reference. An “altered” lytic enzyme can be produced in a number of ways. In an embodiment, a gene for the altered lytic enzyme is put into a transfer or movable vector, preferably a plasmid, and the plasmid is cloned into an expression vector or expression system. The expression vector for producing a lysin polypeptide or enzyme of the invention may be suitable for E. coli, Bacillus, or a number of other suitable bacteria. The vector system may also be a cell free expression system. All of these methods of expressing a gene or set of genes are known in the art.

A “chimeric protein” or “fusion protein” comprises all or (preferably a biologically active) part of a polypeptide of the invention operably linked to a heterologous polypeptide. A relevant biologically active part can be the catalytic domain. A relevant biologically active part can be the binding domain. Chimeric proteins or peptides are produced, for example, by combining two or more proteins having two or more active sites. Chimeric protein and peptides can act independently on the same or different molecules, and hence have a potential to treat two or more different bacterial infections at the same time. Thus a chimeric protein may combine a single binding domain, such as an SH3-type binding domain, with more than one catalytic domain. Chimeric proteins and peptides also may be used to treat a bacterial infection by cleaving the cell wall in more than one location, such as by virtue of two (or more) catalytic domains or two (or more) catalytic activities, thus potentially providing more rapid or effective (or synergistic) killing from a single lysin molecule or chimeric peptide.

A “heterologous” region of a DNA construct or peptide construct is an identifiable segment of DNA within a larger DNA molecule or peptide within a larger peptide molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA or peptide as defined herein.

The term “operably linked” means that the polypeptide of the disclosure and the heterologous polypeptide are fused in-frame. The heterologous polypeptide can be fused to the N-terminus or C-terminus of the polypeptide of the disclosure. Chimeric proteins are produced enzymatically by chemical synthesis, or by recombinant DNA technology. A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetration of the proteins of either the inner membrane or the inner and outer membranes of the E. coli (FEMS Microbiol. Lett. (1998) 164(1):159-67 (incorporated herein by reference). One example of a useful fusion protein is a GST fusion protein in which the polypeptide of the disclosure is fused to the C-terminus of a GST sequence. Such a chimeric protein can facilitate the purification of a recombinant polypeptide of the disclosure.

In another embodiment, the chimeric protein or peptide contains a heterologous signal sequence at its N-terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992, incorporated herein by reference). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The fusion protein may combine a lysin polypeptide with a protein or polypeptide of having a different capability, or providing an additional capability or added character to the lysin polypeptide. The fusion protein may be an immunoglobulin fusion protein in which all or part of a polypeptide of the disclosure is fused to sequences derived from a member of the immunoglobulin protein family. The immunoglobulin may be an antibody, for example an antibody directed to a surface protein or epitope of a susceptible or target bacteria. An immunoglobulin fusion protein can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the disclosure. Inhibition of ligand/receptor interaction may be useful therapeutically, both for treating bacterial-associated diseases and disorders for modulating (i.e. promoting or inhibiting) cell survival. Moreover, an immunoglobulin fusion protein of the disclosure can be used as an immunogen to produce antibodies directed against a polypeptide of the disclosure in a subject, to purify ligands and in screening assays to identify molecules which inhibit the interaction of receptors with ligands. Chimeric and fusion proteins and peptides of the disclosure can be produced by standard recombinant DNA techniques.

The fusion gene can be synthesized by conventional techniques, including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see, i.e., Ausubel et al., supra). Moreover, many expression vectors are commercially available that already encode a fusion moiety (i.e., a GST polypeptide). A nucleic acid encoding a polypeptide of the invention can be cloned into such an expression vector such that the fusion moiety is linked in-frame to the polypeptide of the invention.

As used herein, shuffled proteins or peptides, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property. This method is described, for example, in Stemmer, U.S. Pat. No. 6,132,970. (Method of shuffling polynucleotides); Kauffman, U.S. Pat. No. 5,976,862 (Evolution via Condon-based Synthesis) and Huse, U.S. Pat. No. 5,808,022 (Direct Codon Synthesis). The contents of these patents are incorporated herein by reference. Shuffling can be used to create a protein that is more active, for instance up to 10 to 100 fold more active than the template protein. The template protein is selected among different varieties of lysin proteins. The shuffled protein or peptides constitute, for example, one or more binding domains and one or more catalytic domains. Each binding or catalytic domain is derived from the same or a different phage or phage protein. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable or incapable of translation into a peptide.

The modified or altered form of the protein or peptides and peptide fragments, as disclosed herein, includes protein or peptides and peptide fragments that are chemically synthesized or prepared by recombinant DNA techniques, or both. These techniques include, for example, chimerization and shuffling. When the protein or peptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

A signal sequence of a polypeptide can facilitate transmembrane movement of the protein and peptides and peptide fragments of the disclosure to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the disclosure can pertain to the described polypeptides having a signal sequence, as well as to the signal sequence itself and to the polypeptide in the absence of the signal sequence (i.e., the cleavage products). A nucleic acid sequence encoding a signal sequence of the disclosure can be operably linked in an expression vector to a protein of interest, such as a protein which is ordinarily not secreted or is otherwise difficult to isolate. The signal sequence directs secretion of the protein, such as from an eukaryotic host into which the expression vector is transformed, and the signal sequence is subsequently or concurrently cleaved. The protein can then be readily purified from the extracellular medium by art-recognized methods. Alternatively, the signal sequence can be linked to a protein of interest using a sequence which facilitates purification, such as with a GST domain.

The present invention also pertains to other variants of the polypeptides of the invention. Such variants may have an altered amino acid sequence which can function as either agonists (mimetics) or as antagonists. Variants can be generated by mutagenesis, i.e., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein. Variants of a protein of the disclosure which function as either agonists (mimetics) or as antagonists can be identified by screening combinatorial libraries of mutants, i.e., truncation mutants, of the protein of the disclosure for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (i.e., for phage display). There are a variety of methods which can be used to produce libraries of potential variants of the polypeptides of the disclosure from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477, all herein incorporated by reference).

In addition, libraries of fragments of the coding sequence of a polypeptide of the disclosure can be used to generate a variegated population of polypeptides for screening and subsequent selection of variants, active fragments or truncations. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest. Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the disclosure (Arkin and Yourvan (1992) Proc. Natl. Acad. Sci. USA 89:7811-7815; Delgrave et al. (1993) Protein Engineering 6(3):327-331) immunologically active portions of a protein or peptide fragment include regions that bind to antibodies that recognize the phage enzyme. In this context, the smallest portion of a protein (or nucleic acid that encodes the protein) according to embodiments is an epitope that is recognizable as specific for the phage that makes the lysin protein. Accordingly, the smallest polypeptide (and associated nucleic acid that encodes the polypeptide) that can be expected to bind a target or receptor, such as an antibody, and is useful for some embodiments may be 8, 9, 10, 11, 12, 13, 15, 20, 25, 30, 35, 40, 45, 50, 55, 60, 65, 75, 85, or 100 amino acids long. Although small sequences as short as 8, 9, 10, 11, 12 or 15 amino acids long reliably comprise enough structure to act as targets or epitopes, shorter sequences of 5, 6, or 7 amino acids long can exhibit target or epitopic structure in some conditions and have value in an embodiment. Thus, the smallest portion of the protein(s) or lysin polypeptides provided herein, including as set out in SEQ ID NOS: 3 or 5-6, includes polypeptides as small as 5, 6, 7, 8, 9, 10, 12, 14 or 16 amino acids long.

Biologically active portions of a protein or peptide fragment of the embodiments, as described herein, include polypeptides comprising amino acid sequences sufficiently identical to or derived from the amino acid sequence of the phage protein of the disclosure, which include fewer amino acids than the full length protein of the phage protein and exhibit at least one activity of the corresponding full-length protein. Typically, biologically active portions comprise a domain or motif with at least one activity of the corresponding protein. A biologically active portion of a protein or protein fragment of the disclosure can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the embodiments.

Homologous proteins and nucleic acids can be prepared that share functionality with such small proteins and/or nucleic acids (or protein and/or nucleic acid regions of larger molecules) as will be appreciated by a skilled artisan. Such small molecules and short regions of larger molecules that may be homologous specifically are intended as embodiments. Preferably the homology of such valuable regions is at least 50%, 65%, 75%, 80%, 85%, and preferably at least 90%, 95%, 97%, 98%, or at least 99% compared to the lysin polypeptides provided herein, including as provided or referenced, including SEQ ID NOs: 3 and 5-6. These percent homology values do not include alterations due to conservative amino acid substitutions.

Two amino acid sequences are “substantially homologous” when at least about 70% of the amino acid residues (preferably at least about 80%, at least about 85%, and preferably at least about 90 or 95%) are identical, or represent conservative substitutions. The sequences of comparable lysins, such as comparable PlySs2 lysins, or comparable Sal or LysK lysins, are substantially homologous when one or more, or several, or up to 10%, or up to 15%, or up to 20% of the amino acids of the lysin polypeptide are substituted with a similar or conservative amino acid substitution, and wherein the comparable lysins have the profile of activities, anti-bacterial effects, and/or bacterial specificities of a lysin, such as the PlySs2 and/or Sal or LysK lysins, disclosed herein.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

Mutations can be made in the amino acid sequences, or in the nucleic acid sequences encoding the polypeptides and lysins herein, including in the lysin sequences provided or referenced herein, or in active fragments or truncations thereof, such that a particular codon is changed to a codon which codes for a different amino acid, an amino acid is substituted for another amino acid, or one or more amino acids are deleted. Such a mutation is generally made by making the fewest amino acid or nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

Thus, one of skill in the art, based on a review of the sequence of the lysin polypeptides provided herein, particularly the lysin polypeptides having an SH-3 type binding domain provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for killing bacteria, including Staphylococcal, Streptococcal, Listeria, or Enterococcal bacteria, and/or for having comparable activity to the lysin(s) provided herein. Thus, changes can be made to the sequence of a lysin polypeptide of the invention having an SH-3 type binding domain, including PlySs2 (CF-301), Sal, LysK, and others provided and referenced herein, for example, by modifying the amino acid sequence as set out or referenced herein, and mutants or variants having a change in sequence can be tested using the assays and methods described and exemplified herein, including in the examples. One of skill in the art, on the basis of the domain structure of the lysin(s) hereof can predict one or more, one or several amino acids suitable for substitution or replacement and/or one or more amino acids which are not suitable for substitution or replacement, including reasonable conservative or non-conservative substitutions.

In this regard, and with exemplary reference to PlySs2 (CF-301) lysin but without limitation thereto, it is pointed out that, the PlySs2 (CF-301) polypeptide lysin comprises an N-terminal CHAP domain (cysteine-histidine amidohydrolase/peptidase) and a C-terminal SH3-type 5 domain as depicted herein. The domains are depicted with the CHAP domain corresponding to the first amino acid sequence region starting with LNNV . . . and ending with . . . HYIT, and the SH-3 type domain corresponding to the second region starting with RSYR . . . and ending with . . . YVAT. Similarly relevant N-terminal catalytic and/or C-terminal binding domains, particularly SH-3 type binding domains, in the lysin polypeptides referenced herein and of use in the present invention, including but not limited to Sal lysin, LysK lysin, lysostaphin and also phill lysin, LysH5 lysin, MV-L lysin, LysGH15 lysin, and ALE-1 lysin may be readily identified. CHAP domains are included in several previously characterized streptococcal and staphylococcal phage lysins. Thus, one of skill in the art can reasonably make and test substitutions or replacements to the CHAP domain and/or the SH-3 domain of PlySs2 (CF-301). Sequence comparisons to the Genbank database can be made with either or both of the CHAP and/or SH-3 domain sequences or with the PlySs2 (CF-301) lysin full amino acid sequence, for instance, to identify amino acids for substitution. The CHAP domain, for example, includes conserved cysteine and histidine amino acid sequences (underlined in the PlySs2 (CF-301) sequence herein). It is reasonable to predict, for example, that the conserved cysteine and histidine residues should be maintained in a mutant or variant of PlySs2 (CF-301) so as to maintain activity or capability. It is notable that a mutant or variant having an alanine replaced for valine at valine amino acid 19 in the PlySs2 (CF-301) sequence is active and capable of killing gram positive bacteria in a manner similar to and as effective as originally isolated and sequenced PlySs2 (CF-301) lysin.

For example, PlySs2 (CF-301) lysin (SEQ ID NO:3) comprises an N-terminal CHAP domain (LNNV . . . HYIT; amino acids 8 through 146) and C-terminal SH3 domain (RSYR . . . YVAT; amino acids 162 through 228). Together, these two regions of domain sequence homology constitute 206 of the total 245 amino acids of the PlySs2 (CF-301) amino acid sequence (SEQ ID NO:3), representing 84% of the polypeptide sequence. Thus, much of the PlySs2 (CF-301) lysin amino acid sequence corresponds to domain homologous sequences. Also, structure/function information regarding each of the CHAP and SH3 domain sequences and contributing to semi-rational design of variants, is available to the skilled artisan. For instance, Bateman and Rawlings (Bateman, A. and Rawlings N. D. (2003) Trends in Biochemical Sciences 28(5):234-237 “The CHAP domain: a large family of amidases including GSP amidase and peptidoglycan hydrolases”) describes and identifies the CHAP domain in numerous polypeptides, demonstrating sequence variation in an exemplary alignment and identifying critical invariant cysteine and histidine residues. This analysis and information is expanded by Zou and Hou (Zou, Y. and Hou, C. (2010) Computational Biology and Chemistry 34:251-257 “Systematic analysis of an amidase domain CHAP in 12 Staphylococcus aureus genomes and 44 staphylococcal phage genomes”) in a detailed systematic analysis of CHAP domains in over 50 bacterial and phage genomes, including sequence alignment, consensus secondary structures, analysis of sequence variation, and characterization of highly conserved residues and a sequence signature. Prokaryotic or bacterial SH3 domains were described and characterized in the published art, including structural characterization and identification of well conserved residues and charged residues, hydrophobic residues, etc. For example, Whisstock, J. C. and Lesk, A. M. (“SH3 domains in prokaryotes” Trends in Biochemical Sciences 24:132-133 (1999)) describes SH3 domain homology in bacteria and aligns amino acid sequences denoting conserved residues and aspects. Following this publication, Ponting et al (“Eukaryotic Signaling Domain Homologues in Archae and Bacteria. Ancient Ancestry and Horizontal Gene Transfer” J Mol Biol (1999) 289:729-745) evaluated various domain homologues, including SH3, and provide expanded sequence assessment and alignment across numerous bacterial SH3b domain sequences, describing exemplary substitutions and highlighting well conserved amino acids.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at pH 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces 0-turns in the protein's structure.

A polypeptide or epitope as described herein may be used to generate an antibody and also can be used to detect binding to the lysin or to molecules that recognize the lysin protein. Another embodiment is a molecule such as an antibody or other specific binder that may be created through use of an epitope such as by regular immunization or by a phase display approach where an epitope can be used to screen a library if potential binders. Such molecules recognize one or more epitopes of lysin protein or a nucleic acid that encodes lysin protein. An antibody that recognizes an epitope may be a monoclonal antibody, a humanized antibody, or a portion of an antibody protein. Desirably the molecule that recognizes an epitope has a specific binding for that epitope which is at least 10 times as strong as the molecule has for serum albumin. Specific binding can be measured as affinity (Km). More desirably the specific binding is at least 10², 10³, 10⁴, 10⁵, 10⁶, 10⁷, 10⁸, or even higher than that for serum albumin under the same conditions.

In a desirable embodiment the antibody or antibody fragment is in a form useful for detecting the presence of the lysin protein or, alternatively detecting the presence of a bacteria susceptible to the lysin protein. In a further embodiment the antibody may be attached or otherwise associated with the lysin polypeptide of the invention, for example in a chimeric or fusion protein, and may serve to direct the lysin to a bacterial cell or strain of interest or target. Alternatively, the lysin polypeptide may serve to direct the antibody or act in conjunction with the antibody, for example in lysing the bacterial cell wall fully or partially, so that the antibody may specifically bind to its epitope at the surface or under the surface on or in the bacteria. For example, a lysin of the invention may be attached to an anti-Streptococcal antibody and direct the antibody to its epitope.

A variety of forms and methods for antibody synthesis are known as will be appreciated by a skilled artisan. The antibody may be conjugated (covalently complexed) with a reporter molecule or atom such as a fluor, an enzyme that creates an optical signal, a chemilumiphore, a microparticle, or a radioactive atom. The antibody or antibody fragment may be synthesized in vivo, after immunization of an animal, for example, the antibody or antibody fragment may be synthesized via cell culture after genetic recombination. The antibody or antibody fragment may be prepared by a combination of cell synthesis and chemical modification.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); and (xii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin-binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′)2 and F(v), which portions are preferred for use in the therapeutic methods described herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The term “specific” may be used to refer to the situation in which one member of a specific binding pair will not show significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention hereof, those of skill in the art will appreciate that minor modifications to the N- or C-terminal of the peptide may however be contemplated, such as the chemical modification of the terminal to add a protecting group or the like, e.g. the amidation of the C-terminus.

The term “isolated” refers to the state in which the lysin polypeptide(s) of the invention, or nucleic acid encoding such polypeptides will be, in accordance with the present invention. Polypeptides and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Polypeptides and nucleic acid may be formulated with diluents or adjuvants and still for practical purposes be isolated—for example the polypeptides will normally be mixed with polymers or mucoadhesives or other carriers, or will be mixed with pharmaceutically acceptable carriers or diluents, when used in diagnosis or therapy.

### Nucleic Acids

Nucleic acids capable of encoding the lysin polypeptide(s) of the invention are referenced or provided herein or constitute an aspect of the invention. Representative nucleic acid sequences in this context are polynucleotide sequences coding for the polypeptide of any lysin provided or referenced herein, and sequences that hybridize, under stringent conditions, with complementary sequences of the DNA of the encoding sequence. Further variants of these sequences and sequences of nucleic acids that hybridize with those also are contemplated for use in production of lysing enzymes according to the disclosure, including natural variants that may be obtained. A large variety of isolated nucleic acid sequences or cDNA sequences that encode phage associated lysing enzymes and partial sequences that hybridize with such gene sequences are useful for recombinant production of the lysin enzyme(s) or polypeptide(s) of the invention.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′(amino) terminus and a translation stop codon at the 3′(carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Many of the herein contemplated variant DNA molecules include those created by standard DNA mutagenesis techniques, such as M13 primer mutagenesis. Details of these techniques are provided in Sambrook et al. (1989) In Molecular Cloning: A Laboratory Manual, Cold Spring Harbor, N.Y. (incorporated herein by reference). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the lysin polypeptide(s) are contemplated by the disclosure. Also included are small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of a lytic enzyme genetically coded for by a bacteriophage of Staphylococcus suis and, for the purposes of PCR, will comprise at least a 10-15 nucleotide sequence and, more preferably, a 15-30 nucleotide sequence of the gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989), In Molecular Cloning: A Laboratory Manual, Cold Spring Harbor, N.Y., chapters 9 and 11 (herein incorporated by reference).

An example of such calculation is as follows. A hybridization experiment may be performed by hybridization of a DNA molecule (for example, a natural variation of the lytic enzyme genetically coded for by a bacteriophage specific for Bacillus anthracis) to a target DNA molecule. A target DNA may be, for example, the corresponding cDNA which has been electrophoresed in an agarose gel and transferred to a nitrocellulose membrane by Southern blotting (Southern (1975). J. Mol. Biol. 98:503), a technique well known in the art and described in Sambrook et al. (1989) In Molecular Cloning: A Laboratory Manual, Cold Spring Harbor, N.Y. (incorporated herein by reference). Hybridization with a target probe labeled with isotopic P³² labeled-dCTP is carried out in a solution of high ionic strength such as 6 times SSC at a temperature that is 20-25 degrees Celsius below the melting temperature, Tm (described infra). For such Southern hybridization experiments where the target DNA molecule on the Southern blot contains 10 ng of DNA or more, hybridization is carried out for 6-8 hours using 1-2 ng/ml radiolabeled probe (of specific activity equal to 109 CPM/mug or greater). Following hybridization, the nitrocellulose filter is washed to remove background hybridization. The washing conditions are as stringent as possible to remove background hybridization while retaining a specific hybridization signal. The term “Tm” represents the temperature above which, under the prevailing ionic conditions, the radiolabeled probe molecule will not hybridize to its target DNA molecule. The Tm of such a hybrid molecule may be estimated from the following equation: Tₘ=81.5° C.−16.6(log 10 of sodium ion concentration)+0.41(% G+C)−0.63(% formamide)−(600/l) where l=the length of the hybrid in base pairs. This equation is valid for concentrations of sodium ion in the range of 0.01M to 0.4M, and it is less accurate for calculations of Tm in solutions of higher sodium ion concentration (Bolton and McCarthy (1962). Proc. Natl. Acad. Sci. USA 48:1390) (incorporated herein by reference). The equation also is valid for DNA having G+C contents within 30% to 75%, and also applies to hybrids greater than 100 nucleotides in length. The behavior of oligonucleotide probes is described in detail in Ch. 11 of Sambrook et al. (1989), In Molecular Cloning: A Laboratory Manual, Cold Spring Harbor, N.Y. (incorporated herein by reference). The preferred exemplified conditions described here are particularly contemplated for use in selecting variations of the lytic gene.

In preferred embodiments of the present disclosure, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. Preferably, stringent conditions are those under which DNA sequences with more than 6% mismatch will not hybridize.

The degeneracy of the genetic code further widens the scope of the embodiments as it enables major variations in the nucleotide sequence of a DNA molecule while maintaining the amino acid sequence of the encoded protein. For example, a representative amino acid residue is alanine. This may be encoded in the cDNA by the nucleotide codon triplet GCT. Because of the degeneracy of the genetic code, three other nucleotide codon triplets—GCT, GCC and GCA—also code for alanine. Thus, the nucleotide sequence of the gene could be changed at this position to any of these three codons without affecting the amino acid composition of the encoded protein or the characteristics of the protein. The genetic code and variations in nucleotide codons for particular amino acids are well known to the skilled artisan. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein comprehended by this disclosure.

Thus, it should be appreciated that also within the scope of the present invention are DNA sequences encoding a lysin of the present invention, including PlySs2, which sequences code for a polypeptide having the same amino acid sequence as provided or referenced herein, but which are degenerate thereto or are degenerate to the exemplary nucleic acids sequences provided or referenced. By “degenerate to” is meant that a different three-letter codon is used to specify a particular amino acid. It is well known in the art that the following codons can be used interchangeably to code for each specific amino acid:

| | |
|---|---|
| Phenylaianine (Phe or F) | UUU or UUC |
| Leucine (Leu or L) | UUA or UUG or CUU or CUC or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or AGC |
| Proline (Pro or P) | CCU or CCC or CCA or CCG |
| Threonine (Thr or T) | ACU or ACC or ACA or ACG |
| Martine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or AGG |
| Glycine (Gly or G) | GGU or GGC or GGA or GGG |
| Tryptophan (Trp or W) | UGG |
| Termination codon | UAA (ochre) or UAG (amber) or UGA Tap |

It should be understood that the codons specified above are for RNA sequences. The corresponding codons for DNA have a T substituted for U.

One skilled in the art will recognize that the DNA mutagenesis techniques described here and known in the art can produce a wide variety of DNA molecules that code for a bacteriophage lysin of Streptococcus as an example yet that maintain the essential characteristics of the lytic polypeptides described and provided herein. Newly derived proteins may also be selected in order to obtain variations on the characteristic of the lytic polypeptide(s), as will be more fully described below. Such derivatives include those with variations in amino acid sequence including minor deletions, additions and substitutions.

While the site for introducing an amino acid sequence variation may be predetermined, the mutation per se does not need to be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues, or can be of one or more, one or a few, one, two, three, four, five, six or seven residues; insertions usually will be on the order of about from 1 to 10 amino acid residues; and deletions will range about from 1 to 30 residues. Deletions or insertions may be in single form, but preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure.

Substitutional variants are those in which at least one residue in the amino acid sequence has been removed and a different residue inserted in its place. Such substitutions may be made so as to generate no significant effect on the protein characteristics or when it is desired to finely modulate the characteristics of the protein. Amino acids which may be substituted for an original amino acid in a protein and which are regarded as conservative substitutions are described above and will be recognized by one of skill in the art.

Substantial changes in function or immunological identity may be made by selecting substitutions that are less conservative, for example by selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions which in general are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives or variants of the lytic polypeptide(s) by analyzing the ability of the derivative or variant proteins to lyse or kill susceptible bacteria, or to complement the sensitivity to DNA cross-linking agents exhibited by phages in infected bacteria hosts. These assays may be performed by transfecting DNA molecules encoding the derivative or variant proteins into the bacteria as described above or by incubating bacteria with expressed proteins from hosts transfected with the DNA molecules encoding the derivative or variant proteins.

While the site for introducing an amino acid sequence variation can be predetermined, the mutation per se does not need to be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host. Such operative linking of a DNA sequence of this invention to an expression control sequence, of course, includes, if not already part of the DNA sequence, the provision of an initiation codon, ATG, in the correct reading frame upstream of the DNA sequence. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids colE1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2□ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast □-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention.

Libraries of fragments of the coding sequence of a polypeptide can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein (Arkin and Yourvan (1992) Proc. Natl. Acad. Sci. USA 89:7811-7815; Delgrave et al. (1993) Protein Engineering 6(3):327-331).

### Compositions

Therapeutic or pharmaceutical compositions comprising the lytic enzyme(s)/polypeptide(s) of the invention are provided in accordance with the invention, as well as related methods of use and methods of manufacture. Therapeutic or pharmaceutical compositions may comprise one or more lytic polypeptide(s), and optionally include natural, truncated, chimeric or shuffled lytic enzymes, optionally combined with other components such as a carrier, vehicle, polypeptide, polynucleotide, holin protein(s), one or more antibiotics or suitable excipients, carriers or vehicles. The invention provides therapeutic compositions or pharmaceutical compositions of the lysins of the invention, particularly a lysin having an SH3-type binding domain, including PlySs2 (CF-301), Sal lysin, LysK lysin, lysostaphin, phill lysin, LysH5 lysin, MV-L lysin, LysGH15 lysin, and ALE-1 lysin, for use in the killing, alleviation, decolonization, prophylaxis or treatment of gram-positive bacteria, particularly including Staphylococcus bacteria, including bacterial infections or related conditions. The invention provides therapeutic compositions or pharmaceutical compositions of the lysins of the invention, particularly a lysin having an SH3-type binding domain, including PlySs2 (CF-301), Sal lysin, LysK lysin, lysostaphin, phill lysin, LysH5 lysin, MV-L lysin, LysGH15 lysin, and ALE-1 lysin, for use in treating, reducing or controlling contamination and/or infections by gram positive bacteria, particularly including Streptococcus, including in contamination or infection of or via an external surface such as skin. Compositions are thereby contemplated and provided for topical or dermatological applications and general administration to the exterior, including the skin or other external surface. Compositions comprising a lysin having an SH3-type binding domain, including PlySs2 (CF-301), Sal lysin, LysK lysin, lysostaphin, phill lysin, LysH5 lysin, MV-L lysin, LysGH15 lysin, and ALE-1 lysin, particularly PlySs2 (CF-301), including domains, truncations or variants thereof, are provided herein for use in the killing, alleviation, decolonization, prophylaxis or treatment of gram-positive bacteria, including bacterial infections or related conditions, particularly of Streptococcus, Staphylococcus, Enterococcus or Listeria, including Streptococcus pyogenes and antibiotic resistant Staphylococcus aureus.

The enzyme(s) or polypeptide(s) included in the therapeutic compositions may be one or more or any combination of unaltered phage associated lytic enzyme(s), truncated lytic polypeptides, variant lytic polypeptide(s), and chimeric and/or shuffled lytic enzymes. Additionally, different lytic polypeptide(s) genetically coded for by different phage for treatment of the same bacteria may be used. These lytic enzymes may also be any combination of “unaltered” lytic enzymes or polypeptides, truncated lytic polypeptide(s), variant lytic polypeptide(s), and chimeric and shuffled lytic enzymes. The lytic enzyme(s)/polypeptide(s) in a therapeutic or pharmaceutical composition for gram-positive bacteria, including Streptococcus, Staphylococcus, Enterococcus and/or Listeria, may be used alone or in combination with antibiotics or, if there are other invasive bacterial organisms to be treated, in combination with other phage associated lytic enzymes specific for other bacteria being targeted. The lytic enzyme, truncated enzyme, variant enzyme, chimeric enzyme, chimeric polypeptide, fusion polypeptide, SH-3 binding domain containing peptide or construct, and/or shuffled lytic enzyme may be used in conjunction with another therapeutic or antibacterial peptide. The amount of the another therapeutic or antibacterial peptide may also be varied. Various antibiotics may be optionally included in the therapeutic composition with the enzyme(s) or polypeptide(s) and with or without the presence of another therapeutic or antibacterial peptide. More than one lytic enzyme or polypeptide may be included in the therapeutic composition.

The pharmaceutical composition can also include one or more altered lytic enzymes, including isozymes, analogs, or variants thereof produced by chemical synthesis or DNA recombinant techniques. In particular, altered lytic protein can be produced by amino acid substitution, deletion, truncation, chimerization, fusion, shuffling, or combinations thereof. The pharmaceutical composition may contain a combination of one or more natural lytic protein and one or more truncated, variant, chimeric or shuffled lytic protein. The pharmaceutical composition may also contain a peptide or a peptide fragment of at least one lytic protein derived from the same or different bacteria species, with an optional addition of one or more complementary agent, and a pharmaceutically acceptable carrier or diluent.

The present invention provides bacterial lysins comprising a lytic polypeptide variant, such as a variant of PlySs2 (CF-301), Sal lysin, LysK lysin, lysostaphin, phill lysin, LysH5 lysin, MV-L lysin, LysGH15 lysin, and ALE-1 lysin, particularly such as a PlySs2 (CF-301) lysin polypeptide variant, having bacterial killing activity. In an aspect a variant has an amino acid sequence comprising or having at least 80%, 85%, 90%, 95% or 99% amino acid identity to a lysin polypeptide amino acid sequence provided herein, including to any or one of SEQ ID NOs: 3-7 or any lysin polypeptide sequence referenced herein or provided herein by reference, particularly a reference lysin having an SH-3 type binding domain. The invention includes SH-3 type lysin polypeptide truncation mutants, including PlySs2 (CF-301) lysin truncation mutants, that contain only the binding domain, or that contain only one catalytic or enzymatic domain and retain bacterial binding activity or gram positive antibacterial activity. The invention includes, for example, exemplary lysin truncation mutants that contain only one domain selected from a binding domain, an amidase domain and glucosaminidase domain. In a truncation mutant, for example, an enzymatic domain, such as a glucosaminidase domain is deleted, so that the truncated lysin comprises and contains replacement or alternative N-terminal enzymatic domain and a cell-wall binding domain, particularly an SH3B type binding domain.

The pharmaceutical composition can contain a complementary agent, including one or more antimicrobial agent and/or one or more conventional antibiotics. In order to accelerate treatment of the infection, the therapeutic agent may further include at least one complementary agent which can also potentiate the bactericidal activity of the lytic enzyme. Antimicrobials act largely by interfering with the structure or function of a bacterial cell by inhibition of cell wall synthesis, inhibition of cell-membrane function and/or inhibition of metabolic functions, including protein and DNA synthesis. Antibiotics can be subgrouped broadly into those affecting cell wall peptidoglycan biosynthesis and those affecting DNA or protein synthesis in gram positive bacteria. Cell wall synthesis inhibitors, including penicillin and antibiotics like it, disrupt the rigid outer cell wall so that the relatively unsupported cell swells and eventually ruptures. Antibiotics affecting cell wall peptidoglycan biosynthesis include: Glycopeptides, which inhibit peptidoglycan synthesis by preventing the incorporation of N-acetylmuramic acid (NAM) and N-acetylglucosamine (NAG) peptide subunits into the peptidoglycan matrix. Available glycopeptides include vancomycin and teicoplanin; Penicillins, which act by inhibiting the formation of peptidoglycan cross-links. The functional group of penicillins, the β-lactam moiety, binds and inhibits DD-transpeptidase that links the peptidoglycan molecules in bacteria. Hydrolytic enzymes continue to break down the cell wall, causing cytolysis or death due to osmotic pressure. Common penicillins include oxacillin, ampicillin and cloxacillin; and Polypeptides, which interfere with the dephosphorylation of the C₅₅-isoprenyl pyrophosphate, a molecule that carries peptidoglycan building-blocks outside of the plasma membrane. A cell wall-impacting polypeptide is bacitracin.

The complementary agent may be an antibiotic, such as erythromycin, clarithromycin, azithromycin, roxithromycin, other members of the macrolide family, penicillins, cephalosporins, and any combinations thereof in amounts which are effective to synergistically enhance the therapeutic effect of the lytic enzyme. Virtually any other antibiotic may be used with the altered and/or unaltered lytic enzyme. Similarly, other lytic enzymes may be included in the carrier to treat other bacterial infections. Antibiotic supplements may be used in virtually all uses of the enzyme when treating different diseases.

Also provided are compositions containing nucleic acid molecules that, either alone or in combination with other nucleic acid molecules, are capable of expressing an effective amount of a lytic polypeptide(s) or a peptide fragment of a lytic polypeptide(s) in vivo. Cell cultures containing these nucleic acid molecules, polynucleotides, and vectors carrying and expressing these molecules in vitro or in vivo, are also provided.

Therapeutic or pharmaceutical compositions may comprise lytic polypeptide(s) combined with a variety of carriers to treat the illnesses caused by the susceptible gram-positive bacteria. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl₂, CaCl₂), and others. Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% more preferably about 20%. DMSO is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of 0.1 to 100% (v/v). The carrier vehicle may also include Ringer's solution, a buffered solution, and dextrose solution, particularly when an intravenous solution is prepared.

Any of the carriers for the lytic polypeptide(s) may be manufactured by conventional means. However, it is preferred that any mouthwash or similar type products not contain alcohol to prevent denaturing of the polypeptide/enzyme. Similarly, when the lytic polypeptide(s) is being placed in a cough drop, gum, candy or lozenge during the manufacturing process, such placement should be made prior to the hardening of the lozenge or candy but after the cough drop or candy has cooled somewhat, to avoid heat denaturation of the enzyme.

A lytic polypeptide(s) may be added to these substances in a liquid form or in a lyophilized state, whereupon it will be solubilized when it meets body fluids such as saliva. The polypeptide(s)/enzyme may also be in a micelle or liposome.

The effective dosage rates or amounts of an altered or unaltered lytic enzyme/polypeptide(s) to treat the infection will depend in part on whether the lytic enzyme/polypeptide(s) will be used therapeutically or prophylactically, the duration of exposure of the recipient to the infectious bacteria, the size and weight of the individual, etc. The duration for use of the composition containing the enzyme/polypeptide(s) also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme believed to provide for an effective amount or dosage of enzyme may be in the range of about 100 units/ml to about 500,000 units/ml of fluid in the wet or damp environment of the nasal and oral passages, and possibly in the range of about 100 units/ml to about 50,000 units/ml. More specifically, time exposure to the active enzyme/polypeptide(s) units may influence the desired concentration of active enzyme units per ml. Carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depend on the nature of infection, whether treatment is to be prophylactic or therapeutic, and other variables. There are situations where it may be necessary to have a much higher unit/ml dosage or a lower unit/ml dosage.

The lytic enzyme/polypeptide(s) should be in an environment having a pH which allows for activity of the lytic enzyme/polypeptide(s). For example if a human individual has been exposed to another human with a bacterial upper respiratory disorder, the lytic enzyme/polypeptide(s) will reside in the mucosal lining and prevent any colonization of the infecting bacteria. Prior to, or at the time the altered lytic enzyme is put in the carrier system or oral delivery mode, it is preferred that the enzyme be in a stabilizing buffer environment for maintaining a pH range between about 4.0 and about 9.0, more preferably between about 5.5 and about 7.5.

A stabilizing buffer may allow for the optimum activity of the lysin enzyme/polypeptide(s). The buffer may contain a reducing reagent, such as dithiothreitol. The stabilizing buffer may also be or include a metal chelating reagent, such as ethylenediaminetetracetic acid disodium salt, or it may also contain a phosphate or citrate-phosphate buffer, or any other buffer. The DNA coding of these phages and other phages may be altered to allow a recombinant enzyme to attack one cell wall at more than two locations, to allow the recombinant enzyme to cleave the cell wall of more than one species of bacteria, to allow the recombinant enzyme to attack other bacteria, or any combinations thereof. The type and number of alterations to a recombinant bacteriophage produced enzyme are incalculable.

A mild surfactant can be included in a therapeutic or pharmaceutical composition in an amount effective to potentiate the therapeutic effect of the lytic enzyme/polypeptide(s) may be used in a composition. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

Preservatives may also be used in this invention and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Pharmaceuticals for use in all embodiments of the invention include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%.

Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide are recommended at concentrations of about 0.01% to 1.0% by weight. Preferred concentrations for corticosteroids such as hydrocortisone or methylprednisolone acetate are from about 0.2% to about 5.0% by weight.

Additionally, the therapeutic composition may further comprise other enzymes, such as the enzyme lysostaphin for the treatment of any Staphylococcus aureus bacteria present along with the susceptible gram-positive bacteria. Mucolytic peptides, such as lysostaphin, have been suggested to be efficacious in the treatment of S. aureus infections of humans (Schaffner et al., Yale J. Biol. & Med., 39:230 (1967). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the altered lytic enzyme, can be used in the presence or absence of antibiotics. There is a degree of added importance in using both lysostaphin and the lysin enzyme in the same therapeutic agent. Frequently, when a human has a bacterial infection, the infection by one genus of bacteria weakens the human body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other Gram positive bacterial strains will not be killed by standard antibiotics. Consequently, the use of the lysin and lysostaphin, possibly in combination with antibiotics, can serve as the most rapid and effective treatment of bacterial infections. A therapeutic composition may also include mutanolysin, and lysozyme.

Means of application of the therapeutic composition comprising a lytic enzyme/polypeptide(s) include, but are not limited to direct, indirect, carrier and special means or any combination of means. Direct application of the lytic enzyme/polypeptide(s) may be by any suitable means to directly bring the polypeptide in contact with the site of infection or bacterial colonization, such as to the nasal area (for example nasal sprays), dermal or skin applications (for example topical ointments or formulations), suppositories, tampon applications, etc. Nasal applications include for instance nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application. The forms in which the lytic enzyme may be administered include but are not limited to lozenges, troches, candies, injectants, chewing gums, tablets, powders, sprays, liquids, ointments, and aerosols.

When the natural and/or altered lytic enzyme(s)/polypeptide(s) is introduced directly by use of sprays, drops, ointments, washes, injections, packing and inhalers, the enzyme is preferably in a liquid or gel environment, with the liquid acting as the carrier. A dry anhydrous version of the altered enzyme may be administered by the inhaler and bronchial spray, although a liquid form of delivery is preferred.

Compositions for treating topical infections or contaminations comprise an effective amount of at least one lytic enzyme, including PlySs2 (CF-301), Sal lysin, LysK lysin, lysostaphin, phill lysin, LysH5 lysin, MV-L lysin, LysGH15 lysin, and ALE-1 lysin, particularly including PlySs2 (CF-301), according to the invention and a carrier for delivering at least one lytic enzyme to the infected or contaminated skin, coat, or external surface of a mammal, a human, a companion animal or livestock. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes, but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is most effective for the treatment of infected skin. The carriers of topical compositions may comprise semi-solid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament. Polymer thickeners that may be used include those known to one skilled in the art, such as hydrophilic and hydroalcoholic gelling agents frequently used in the cosmetic and pharmaceutical industries. CARBOPOL® is one of numerous crosslinked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. KLUCEL® is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

A composition comprising a lytic enzyme/polypeptide(s) can be administered in the form of a candy, chewing gum, lozenge, troche, tablet, a powder, an aerosol, a liquid, a liquid spray, or toothpaste for the prevention or treatment of bacterial infections associated with upper respiratory tract illnesses. The lozenge, tablet, or gum into which the lytic enzyme/polypeptide(s) is added may contain sugar, corn syrup, a variety of dyes, non-sugar sweeteners, flavorings, any binders, or combinations thereof. Similarly, any gum-based products may contain acacia, carnauba wax, citric acid, cornstarch, food colorings, flavorings, non-sugar sweeteners, gelatin, glucose, glycerin, gum base, shellac, sodium saccharin, sugar, water, white wax, cellulose, other binders, and combinations thereof. Lozenges may further contain sucrose, cornstarch, acacia, gum tragacanth, anethole, linseed, oleoresin, mineral oil, and cellulose, other binders, and combinations thereof. Sugar substitutes can also be used in place of dextrose, sucrose, or other sugars.

Compositions comprising lytic enzymes, or their peptide fragments can be directed to the mucosal lining, where, in residence, they kill colonizing disease bacteria. The mucosal lining, as disclosed and described herein, includes, for example, the upper and lower respiratory tract, eye, buccal cavity, nose, rectum, vagina, periodontal pocket, intestines and colon. Due to natural eliminating or cleansing mechanisms of mucosal tissues, conventional dosage forms are not retained at the application site for any significant length of time.

It may be advantageous to have materials which exhibit adhesion to mucosal tissues, to be administered with one or more phage enzymes and other complementary agents over a period of time. Materials having controlled release capability are particularly desirable, and the use of sustained release mucoadhesives has received a significant degree of attention. J. R. Robinson (U.S. Pat. No. 4,615,697, incorporated herein by reference) provides a good review of the various controlled release polymeric compositions used in mucosal drug delivery. The patent describes a controlled release treatment composition which includes a bioadhesive and an effective amount of a treating agent. The bioadhesive is a water swellable, but water insoluble fibrous, crosslinked, carboxy functional polymer containing (a) a plurality of repeating units of which at least about 80 percent contain at least one carboxyl functionality, and (b) about 0.05 to about 1.5 percent crosslinking agent substantially free from polyalkenyl polyether. While the polymers of Robinson are water swellable but insoluble, they are crosslinked, not thermoplastic, and are not as easy to formulate with active agents, and into the various dosage forms, as the copolymer systems of the present application. Micelles and multilamillar micelles may also be used to control the release of enzyme.

Other approaches involving mucoadhesives which are the combination of hydrophilic and hydrophobic materials, are known. Orahesive® from E. R. Squibb & Co is an adhesive which is a combination of pectin, gelatin, and sodium carboxymethyl cellulose in a tacky hydrocarbon polymer, for adhering to the oral mucosa. However, such physical mixtures of hydrophilic and hydrophobic components eventually fall apart. In contrast, the hydrophilic and hydrophobic domains in this application produce an insoluble copolymer. U.S. Pat. No. 4,948,580, also incorporated by reference, describes a bioadhesive oral drug delivery system. The composition includes a freeze-dried polymer mixture formed of the copolymer poly(methyl vinyl ether/maleic anhydride) and gelatin, dispersed in an ointment base, such as mineral oil containing dispersed polyethylene. U.S. Pat. No. 5,413,792 (incorporated herein by reference) discloses paste-like preparations comprising (A) a paste-like base comprising a polyorganosiloxane and a water soluble polymeric material which are preferably present in a ratio by weight from 3:6 to 6:3, and (B) an active ingredient. U.S. Pat. No. 5,554,380 claims a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c) a wax material. U.S. Pat. No. 5,942,243 describes some representative release materials useful for administering antibacterial agents, which are incorporated by reference.

Therapeutic or pharmaceutical compositions can also contain polymeric mucoadhesives including a graft copolymer comprising a hydrophilic main chain and hydrophobic graft chains for controlled release of biologically active agents. The graft copolymer is a reaction product of (1) a polystyrene macromonomer having an ethylenically unsaturated functional group, and (2) at least one hydrophilic acidic monomer having an ethylenically unsaturated functional group. The graft chains consist essentially of polystyrene, and the main polymer chain of hydrophilic monomeric moieties, some of which have acidic functionality. The weight percent of the polystyrene macromonomer in the graft copolymer is between about 1 and about 20% and the weight percent of the total hydrophilic monomer in the graft copolymer is between 80 and 99%, and wherein at least 10% of said total hydrophilic monomer is acidic, said graft copolymer when fully hydrated having an equilibrium water content of at least 90%. Compositions containing the copolymers gradually hydrate by sorption of tissue fluids at the application site to yield a very soft jelly like mass exhibiting adhesion to the mucosal surface. During the period of time the composition is adhering to the mucosal surface, it provides sustained release of the pharmacologically active agent, which is absorbed by the mucosal tissue.

The compositions of this application may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition.

The dosage forms of the compositions of this invention can be prepared by conventional methods. In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. A vasoconstriction agent can be added to the formulation. The pharmaceutical preparations according to this application are provided sterile and pyrogen free.

A lytic enzyme/polypeptide(s) of the invention may also be administered by any pharmaceutically applicable or acceptable means including topically, orally or parenterally. For example, the lytic enzyme/polypeptide(s) can be administered intramuscularly, intrathecally, subdermally, subcutaneously, or intravenously to treat infections by gram-positive bacteria. In cases where parenteral injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. A vasoconstriction agent can be added to the formulation. The pharmaceutical preparations according to this application are provided sterile and pyrogen free.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The effective dosage rates or amounts of the lytic enzyme/polypeptide(s) to be administered parenterally, and the duration of treatment will depend in part on the seriousness of the infection, the weight of the patient, particularly human, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzymes believed to provide for an effective amount or dosage of enzymes may be selected as appropriate. The amount of active units per ml and the duration of time of exposure depend on the nature of infection, and the amount of contact the carrier allows the lytic enzyme(s)/polypeptide(s) to have.

### Methods and Assays

The bacterial killing capability, and indeed the significantly broad range of bacterial killing, exhibited by the lysin polypeptide(s) of the invention provides for various methods based on the antibacterial effectiveness of the polypeptide(s) of the invention. Thus, the present invention contemplates antibacterial methods, including methods for killing of gram-positive bacteria, for reducing a population of gram-positive bacteria, for treating or alleviating a bacterial infection, for treating a human subject exposed to a pathogenic bacteria, and for treating a human subject at risk for such exposure. The susceptible bacteria include the bacteria from which the phage enzyme(s) of the invention are originally derived, and may also include as well various other Streptococcal, Staphylococcal, Enterococcal and/or Listeria bacterial strains. Methods of treating various conditions are also provided, including methods of prophylactic treatment of Streptococcal, Staphylococcal, Enterococcal and/or Listeria infections, treatment of Streptococcal, Staphylococcal, Enterococcal and/or Listeria infections, reducing Streptococcal, Staphylococcal, Enterococcal and/or Listeria population or carriage, treating lower respiratory infection, treating ear infection, treating ottis media, treating endocarditis, and treating or preventing other local or systemic infections or conditions.

The lysin(s) of the present invention demonstrate capability to kill and effectiveness against bacteria from various species such as multiple Streptococcal or Staphylococcal species, bacteria across distinct species groups such as bacteria from each of Streptococcal, Staphylococcal, Enterococcal and/or Listeria, and bacterial from distinct orders. In particular, the lysin(s) of the present invention demonstrate capability to kill and effectiveness against Streptococcal and Staphylococcal bacteria. In particular, the lysin(s) of the present invention demonstrate capability to kill and effectiveness against Staphylococcal bacteria. The PlySs2 (CF-301) lysin is demonstrated to kill bacteria from two distinct orders, particularly Bacillales and Lactobacillales, in vitro and in vivo. The invention thus contemplates treatment, decolonization, and/or decontamination of bacteria, cultures or infections or in instances wherein more than one gram positive bacteria is suspected or present. In particular, the invention contemplates treatment, decolonization, and/or decontamination of bacteria, cultures or infections or in instances wherein more than one type of Bacilalles bacteria, more than one type of Lactobacillales bacteria, or at least one type of Bacillales and one type of Lactobacillales bacteria is suspected, present, or may be present.

This invention may also be used to treat septicemia, particularly in a human. For the treatment of a septicemic infection, such as for pneumoniae, or bacterial meningitis, there should be a continuous intravenous flow of therapeutic agent into the blood stream. The concentration of the enzymes for the treatment of septicemia is dependent upon the bacterial count in the blood and the blood volume.

Also provided is a method for treating Streptococcal, Staphylococcal, Enterococcal and/or Listeria infection, carriage or populations comprises treating the infection with a therapeutic agent comprising an effective amount of at least one lytic enzyme(s)/polypeptide(s) of the invention, particularly a lysin comprising and SH3-type binding domain, particularly PlySs2. Also provided is a method for treating Streptococcal infection or of treating Streptococcal and/or Staphylococcal infection, carriage or populations comprises treating the infection with a therapeutic agent comprising an effective amount of at least one lytic enzyme(s)/polypeptide(s) of the invention, particularly a lysin comprising and SH3-type binding domain, particularly PlySs2 (CF-301) lysin, Sal lysin, LysK lysin, lysostaphin, phill lysin, LysH5 lysin, MV-L lysin, LysGH15 lysin, or ALE-1 lysin, particularly PlySs2 (CF-301). In an aspect, lytic enzyme/polypeptide capable of lysing the cell wall of Streptococcal, Staphylococcal, Enterococcal and/or Listeria bacterial strains is produced or provided. In the methods of the invention, the lysin polypeptide(s) of the present invention, particularly a lysin comprising and SH3-type binding domain, including particularly PlySs2 (CF-301), are useful and capable in prophylactic and treatment methods directed against gram-positive bacteria, particularly selected from Streptococcal, Staphylococcal, Enterococcal and/or Listeria infections, particularly Streptococcal and/or Staphylococcal infections or bacterial colonization. Bacterial strains susceptible and relevant as targets in the methods of the invention include and may be selected from Staphylococcus aureus, Listeria monocytogenes, Staphylococcus simulans, Streptococcus suis, Staphylococcus epidermidis, Streptococcus equi, Streptococcus equi zoo, Streptococcus agalactiae (GBS), Streptococcus pyogenes (GAS), Streptococcus sanguinis, Streptococcus gordonii, Streptococcus dysgalactiae, Group G Streptococcus, Group E Streptococcus, Enterococcus faecalis and Streptococcus pneumonia. In a particular aspect, bacterial strains are selected from Staphylococcus aureus, Staphylococcus simulans, Streptococcus suis, Staphylococcus epidermidis, Streptococcus equi, Streptococcus equi zoo, Streptococcus agalactiae (GBS), Streptococcus pyogenes (GAS), Streptococcus sanguinis, Streptococcus gordonii, Streptococcus dysgalactiae, Group G Streptococcus, Group E Streptococcus and Streptococcus pneumonia.

The invention includes methods of treating or alleviating Streptococcal, including S. pyogenes, and/or Staphylococcal, including S. aureus, related infections or conditions, including antibiotic-resistant Staphylococcus aureus, particularly including MRSA, wherein the bacteria or a human subject infected by or exposed to the particular bacteria, or suspected of being exposed or at risk, is contacted with or administered an amount of isolated lysin polypeptide(s) of the invention effective to kill the particular bacteria. Thus, one or more of particularly a lysin comprising and SH3-type binding domain, particularly selected from PlySs2 (CF-301) lysin, Sal lysin, LysK lysin, lysostaphin, phill lysin, LysH5 lysin, MV-L lysin, LysGH15 lysin, and ALE-1 lysin, particularly PlySs2 (CF-301), including truncations or variants thereof, including such polypeptides as provided and referenced herein, is contacted or administered so as to be effective to kill the relevant bacteria or otherwise alleviate or treat the bacterial infection.

The term ‘agent’ means any molecule, including polypeptides, antibodies, polynucleotides, chemical compounds and small molecules. In particular the term agent includes compounds such as test compounds, added additional compound(s), or lysin enzyme compounds.

The term ‘agonist’ refers to a ligand that stimulates the receptor the ligand binds to in the broadest sense.

The term ‘assay’ means any process used to measure a specific property of a compound. A ‘screening assay’ means a process used to characterize or select compounds based upon their activity from a collection of compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Therapeutically effective amount’ means that amount of a drug, compound, antimicrobial, antibody, polypeptide, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of infection of gram-positive bacteria, including having a bacteriocidal and/or bacteriostatic effect. The phrase “therapeutically effective amount” is used herein to mean an amount sufficient to prevent, and preferably reduce by at least about 30 percent, more preferably by at least 50 percent, most preferably by at least 90 percent, a clinically significant change in the growth or amount of infectious bacteria, or other feature of pathology such as for example, elevated fever or white cell count as may attend its presence and activity.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

It is noted that in the context of treatment methods which are carried out in vivo or medical and clinical treatment methods in accordance with the present application and claims, the term subject, patient or individual is intended to refer to a human.

The terms “gram-positive bacteria”, “Gram-positive bacteria”, “gram-positive” and any variants not specifically listed, may be used herein interchangeably, and as used throughout the present application and claims refer to Gram-positive bacteria which are known and/or can be identified by the presence of certain cell wall and/or cell membrane characteristics and/or by staining with Gram stain. Gram positive bacteria are known and can readily be identified and may be selected from but are not limited to the genera Listeria, Staphylococcus, Streptococcus, Enterococcus, Mycobacterium, Corynebacterium, and Clostridium, and include any and all recognized or unrecognized species or strains thereof. In an aspect of the invention, the PlySs2 (CF-301) lysin sensitive gram-positive bacteria include bacteria selected from one or more of Listeria, Staphylococcus. Streptococcus, and Enterococcus, particularly Streptococcus and Staphylococcus bacteria. LysK and Sal1 lysin sensitive bacteria include Staphylococcus bacteria.

The term “bacteriocidal” refers to capable of killing bacterial cells.

The term “bacteriostatic” refers to capable of inhibiting bacterial growth, including inhibiting growing bacterial cells.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

One method for treating systemic or tissue bacterial infections caused by Streptococcus or Staphylococcus bacteria comprises parenterally treating the infection with a therapeutic agent comprising an effective amount of one or more lysin polypeptide(s) of the invention, particularly a lysin comprising and SH3-type binding domain, selected from PlySs2 (CF-301) lysin, Sal lysin, LysK lysin, lysostaphin, phill lysin, LysH5 lysin, MV-L lysin, LysGH15 lysin, and ALE-1 lysin, particularly PlySs2 (CF-301), including fusions, chimerics, truncations or variants thereof, including such polypeptides as provided herein an appropriate carrier. A number of other different methods may be used to introduce the lytic enzyme(s)/polypeptide(s). These methods include introducing the lytic enzyme(s)/polypeptide(s) intravenously, intramuscularly, subcutaneously, intrathecally, and subdermally. One skilled in the art, including medical personnel, will be capable of evaluating and recognizing the most appropriate mode or means of administration, given the nature and extent of the bacterial condition and the strain or type of bacteria involved or suspected. For instance, intrathecal use and administration of one or more lytic polypeptide(s) would be most beneficial for treatment of bacterial meningitis.

Infections may be also be treated by injecting into the infected tissue of the human patient a therapeutic agent comprising the appropriate lytic enzyme(s)/polypeptide(s) and a carrier for the enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein. Other phage associated lytic enzymes, along with a holin protein, may be included in the composition.

Various methods of treatment are provided for using a lytic enzyme/polypeptide(s), particularly a lysin comprising and SH3-type binding domain, particularly selected from PlySs2 (CF-301) lysin, Sal lysin, LysK lysin, lysostaphin, phill lysin, LysH5 lysin, MV-L lysin, LysGH15 lysin, and ALE-1 lysin, such as PlySs2 (CF-301) as exemplified herein, as a prophylactic treatment for eliminating or reducing the carriage of susceptible bacteria, preventing those humans who have been exposed to others who have the symptoms of an infection from getting sick, or as a therapeutic treatment for those who have already become ill from the infection. Similarly, the lytic enzyme(s)/polypeptide(s) can be used to treat, for example, lower respiratory tract illnesses, particularly by the use of bronchial sprays or intravenous administration of the enzyme. For example, a lytic enzyme can be used for the prophylactic and therapeutic treatment of eye infections, such as conjunctivitis. The method of treatment comprises administering eye drops or an eye wash which comprise an effective amount of at least one lytic polypeptide(s) of the invention and a carrier capable of being safely applied to an eye, with the carrier containing the lytic enzymes. The eye drops or eye wash are preferably in the form of an isotonic solution. The pH of the solution should be adjusted so that there is no irritation of the eye, which in turn would lead to possible infection by other organisms, and possible to damage to the eye. While the pH range should be in the same range as for other lytic enzymes, the most optimal pH will be in the range as demonstrated and provided herein. Similarly, buffers of the sort described above for the other lytic enzymes should also be used. Other antibiotics which are suitable for use in eye drops may be added to the composition containing the enzymes. Bactericides and bacteriostatic compounds may also be added. The concentration of the enzyme(s) in the solution can be in the range of from about 100 units/ml to about 500,000 units/ml, with a more preferred range of about 100 to about 5,000 units/mil, and about 100 to about 50,000 units/ml. Concentrations can be higher or lower than the ranges provided.

The lytic polypeptide(s) of the invention may also be used in a contact lens solution, for the soaking and cleaning of contact lenses. This solution, which is normally an isotonic solution, may contain, in addition to the enzyme, sodium chloride, mannitol and other sugar alcohols, borates, preservatives, and the like. A lytic enzyme/polypeptide of the invention may also be administered to the ear of a patient. Thus, for instance a lytic polypeptide(s) of the invention may be used to treat ear infections, for example caused by Streptococcus pneumoniae. Otitis media is an inflammation of the middle ear characterized by symptoms such as otalgia, hearing loss and fever. One of the primary causes of these symptoms is a build up of fluid (effusion) in the middle ear. Complications include permanent hearing loss, perforation of the tympanic membrane, acquired cholesteatoma, mastoiditis, and adhesive otitis. Children who develop otitis media in the first years of life are at risk for recurrent acute or chronic disease. One of the primary causes of otitis media is Streptococcus pneumoniae. The lytic enzyme(s)/polypeptide(s) may be applied to an infected ear by delivering the enzyme(s) in an appropriate carrier to the canal of the ear. The carrier may comprise sterile aqueous or oily solutions or suspensions. The lytic enzyme(s) may be added to the carrier, which may also contain suitable preservatives, and preferably a surface-active agent. Bactericidal and fungicidal agents preferably included in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol. Additionally, any number of other eardrop carriers may be used. The concentrations and preservatives used for the treatment of otitis media and other similar ear infections are the same as discussed for eye infections, and the carrier into which the enzyme goes is similar or identical to the carriers for treatment of eye infections. Additionally, the carrier may typically includes vitamins, minerals, carbohydrates, sugars, amino acids, proteinaceous materials, fatty acids, phospholipids, antioxidants, phenolic compounds, isotonic solutions, oil based solutions, oil based suspensions, and combinations thereof.

The diagnostic, prophylactic and therapeutic possibilities and applications that are raised by the recognition of and isolation of the lysin polypeptide(s) of the invention, derive from the fact that the polypeptides of the invention cause direct and specific effects (e.g. killing) in susceptible bacteria. Thus, the polypeptides of the invention may be used to eliminate, characterize, or identify the relevant and susceptible bacteria.

Thus, a diagnostic method of the present invention may comprise examining a cellular sample or medium for the purpose of determining whether it contains susceptible bacteria, or whether the bacteria in the sample or medium are susceptible by means of an assay including an effective amount of one or more lysin polypeptide(s) and a means for characterizing one or more cell in the sample, or for determining whether or not cell lysis has occurred or is occurring. Patients capable of benefiting from this method include those suffering from an undetermined infection, a recognized bacterial infection, or suspected of being exposed to or carrying a particular bacteria. A fluid, food, medical device, composition or other such sample which will come in contact with a subject or patient may be examined for susceptible bacteria or may be eliminated of relevant bacteria. In one such aspect a fluid, food, medical device, composition or other such sample may be sterilized or otherwise treated to eliminate or remove any potential relevant bacteria by incubation with or exposure to one or more lytic polypeptide(s) of the invention.

The procedures and their application are all familiar to those skilled in the art and accordingly may be utilized within the scope of the present invention. In one instance, the lytic polypeptide(s) of the invention complex(es) with or otherwise binds or associates with relevant or susceptible bacteria in a sample and one member of the complex is labeled with a detectable label. The fact that a complex has formed and, if desired, the amount thereof, can be determined by known methods applicable to the detection of labels. The labels most commonly employed for these studies are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. The radioactive label can be detected by any of the currently available counting procedures. The preferred isotope may be selected from ³H, ⁴C, ³²P, ³⁵S, ³⁶C, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

The PlySs2 (CF-301) lysin displays activity and capability to kill numerous distinct strains and species of gram positive bacteria, including Staphylococcal, Streptococcal, Listeria, or Enterococcal bacteria. In particular and with significance, PlySs2 (CF-301) is active in killing Staphylococcus strains, including Staphylococcus aureus, particularly both antibiotic-sensitive and distinct antibiotic-resistant strains. PlySs2 (CF-301) is also active in killing Streptococcus strains, and shows particularly effective killing against Group A and Group B streptococcus strains. PlySs2 (CF-301) lysin capability against bacteria is depicted below in TABLE 1, based on log kill assessments using isolated strains in vitro. The susceptible bacteria provided herein may be used in the modified BMD methods of the invention for determining and comparing MIC values.

TABLE 1
PlySs2 Reduction in Growth of Different Bacteria (partial listing)

| | Relative Kill |
|---|---|
| Bacteria | with PlySs2 |
| Staphylococcus aureus | +++ |
| (VRSA, VISA, MRSA, MSSA) | |
| Streptococcus suis | +++ |
| Staphylococcus epidermidis | ++ |
| Staphylococcus simulans | +++ |
| Lysteria monocytogenes | ++ |
| Enterococcus faecalis | ++ |
| Streptococcus dysgalactiae - GBS | ++ |
| Streptococcus agalactiae -GBS | +++ |
| Streptococcus pyogenes -GAS | +++ |
| Streptococcus equi | ++ |
| Streptococcus sanguinis | ++ |
| Streptococcus gordonii | ++ |
| Streptococcus sobrinus | + |
| Streptococcus rattus | + |
| Streptococcus oralis | + |
| Streptococcus pneumonine | + |
| Bacillus thuringiensis | − |
| Bacillus cereus | − |
| Bacillus subtilis | − |
| Bacillus anthracis | − |
| Escherichia coli | − |
| Enterococcus faecium | − |
| Pseudomonas aeruginosa | − |

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

Bacteriophage-derived lysins are cell wall hydrolytic enzymes that provide an emerging therapeutic option to counter the rise and spread of drug-resistant bacterial pathogens. As purified recombinant proteins, lysins exhibit rapid species-specific bacteriolytic effects, anti-biofilm activity, a low propensity for resistance and pronounced synergy with antibiotics. In the effort to investigate the therapeutic potential of lysins, we have discovered a potent “enhancer effect” exerted by human blood matrices on the antistaphylococcal activity of the lysin PlySs2 (CF-301). The activity of PlySs2 (CF-301) in whole blood, serum and plasma results in a ≥100-fold reduction in minimal sterilizing concentrations (time-kill assay) and a ≥32-fold reduction in minimal inhibitory concentrations (broth microdilution assay) compared to activity in conventional media (cation adjusted Mueller Hinton Broth (caMHB)) across a range of S. aureus strains. The enhancer effect is further increased by synergistic combinations of PlySs2 (CF-301) with either daptomycin or vancomycin. Thus, PlySs2 (CF-301) exhibited substantially greater potency (32-≥100-fold) in human blood compared to caMHB in standard microbiologic testing formats (e.g. MIC, checkerboard and time kill assays).

Enhancer activity was also noted using other anti-staphylococcal lysins. Human, rabbit, horse and dog sera exerted an equivalent enhancer effect on PlySs2 (CF-301) activity, whereas the effect is intermediate in rat and calf and absent in mouse sera. We additionally provide evidence that the mechanism of enhancement involves synergy with at least two blood components, lysozyme and serum albumin, which can be added in multiple assay formats to recapitulate the blood effect. Finally, the predictions based on ex vivo findings were confirmed in vivo by studies showing a superior efficacy profile for PlySs2 (CF-301)(in addition to daptomycin) in the treatment of infective endocarditis in rabbits as compared to rats. Well established rabbit and rat models of S. aureus infectious endocarditis (IE) were used to validate these findings in vivo by demonstrating comparable efficacy at 111-fold lower doses in the rabbit vs the rat model. Overall, these findings suggest that favorable synergistic interactions between PlySs2 (CF-301) and serum proteins act to facilitate bactericidal activity and are expected to have important therapeutic implications.

The rise and spread of drug- and multidrug-resistant bacteria has created a need for novel alternatives or adjunctive therapies to conventional antibiotics. One promising approach now under development is based on the use of recombinantly-produced bacteriophage-derived lysins (cell wall hydrolases) to kill gram-positive bacterial pathogens (1, 2). Lysins are antimicrobial enzymes that provide a novel alternative to conventional antibiotics. Lysins are proteins encoded by bacteriophages and used to kill bacteria in a natural setting. There are about 10³¹ phage in the biosphere and phage kill approximately one-third of all bacteria daily with the lysin protein family the primary means to kill host bacteria (Hatful G F (2015) J Virol 89(16):8107-8110). Purified lysins exhibit the phenomenon called “lysis from without” (Fischetti V A et al (20016) Nature Biotechnology 24:1508-1511) and are amendable to synthetic recombinant manufacture. Purified lysins exhibit potent bacteriolytic effect on contact via cell wall hydrolysis. Lysin polypeptides are typically a 20-30 kDa protein.

PlySs2 lysin, also denoted CF-301, PlySs2 (CF-301), is an antistaphylococcal lysin, and is the first agent of the lysin class to enter Phase 2 of clinical development in the United States for the treatment of bacteremia including endocarditis due to Staphylococcus aureus (3). PlySs2 (CF-301) was originally derived from a prophage carried by Streptococcus suis in pigs. PlySs2 (CF-301) lysin has been demonstrated to kill various strains of clinically significant gram-positive bacteria, including antibiotic resistant strains such as methicillin and vancomycin resistant and sensitive strains of Staphylococcus aureus (MRSA, MSSA, VRSA, VISA), daptomycin-resistant Staphylococcus aureus (DRSA), and linezolid-resistant Staphylococcus aureus (LRSA). PlySs2 (CF-301) has comparatively broad but defined species killing activity and can kill multiple species of bacteria, particularly gram-positive bacteria, including Staphylococcus, Streptococcus, Enterococcus and Listeria bacterial strains, while being inactive against bacteria in the natural intestinal flora.

Clinical grade PlySs2/CF-301 has been produced recombinantly in E. coli and is active over broad pH (pH 6-9.7) and temperature (16-55° C.) ranges (Gilmer et al (2013) Antimicrob Agents Chemother 57:2743-2750; Scuch et al (2014) J Infect Dis 209:1469-78). It is active in various human matrices including blood, serum, plasma, saliva, synovial fluid, pulmonary surfactant and bronchial lavage fluid. The amino acid sequence and structure of PlySs2 (CF-301) is provided above herein.

PlySs2 (CF-301) targets the cell wall of sensitive bacteria, including Staphylococcus aureus. It is a cysteine-histidine aminopeptidase that targets the D-Ala-L-Gly bond in the cell wall peptidoglycan and cleaves between D-alanine (stem peptide) and L-glycine (cross-bridge) of the cell wall. Bacterial lysis is rapid. PlySs2 (CF-301) has defined species specificity and kills antibiotic resistant bacteria including MSSA, MRSA, VRSA, DRSA and LRSA, bacteria resistant to methicillin, vancomycin, daptomycin, linezolid antibiotics (Schuch R et al (2014) J Infect Dis 209:1469-1478). Killing is rapid and potent and a low resistance profile to the lytic peptide is seen. PlySs2 (CF-301) eradicates biofilms and kills persistent bacteria. Effectiveness against biofilms is described in WO 2013/170022 and U.S. Pat. No. 9,499,594, incorporated herein by reference. Synergy with antibiotics has been observed, including as described in WO 2013/170015, incorporated herein by reference.

Hallmark features of PlySs2 (CF-301) include: (i) a potent, targeted and rapid bacteriolytic effect against a broad range of S. aureus isolates, (ii) anti-biofilm activity, (iii) a low propensity for resistance, and (iv) synergy with conventional antibiotics (4, 5). While PlySs2 (CF-301), and indeed many additional lysins described in the literature, are highly effective bacteriolytic agents in the context of standard in vitro testing media and are highly efficacious in variety of animal models of invasive and topical infections, there is virtually no understanding of lysin activity in complex human physiological fluids, such as whole blood, plasma, and serum.

During the pre-clinical phase of antimicrobial development, initial evaluations of therapeutic potential are based on assays performed using laboratory media to determine minimal inhibitory concentrations (MICs) and assess time-dependent killing in the time-kill assay format (6). However, it is also known that in vivo efficacy (especially for systemically delivered drugs) is influenced by multifactorial interactions with components of the human body, in particular, that of blood. For many antibiotic classes, including β-lactams, quinolones, and cyclic lipopeptides, the correlation between plasma protein binding and diminished (up to 10-fold) efficacy (7-11) that must be accounted for in dosing regimens is very well studied. For example, the binding to blood components such as albumin, α₁-acid glycoprotein, lipoproteins, α-, β-, and γ-globulins, and erythrocytes may decrease the amount of free, active drug. Conversely, there are an increasing number of studies that also demonstrate the ability of human blood matrices to potentiate antibacterial activity (up to 16-fold) of both antibiotics (12, 13) and antimicrobial peptides (13-17). The ability of serum to enhance antimicrobial activity has, for example, been attributed to multiple factors, ranging from an influence on growth rate (7, 18) to synergistic interactions with humoral effectors of the innate and adaptive immune systems (13, 15, 16, 19). The development of agents that may have both a potent (and intrinsic) antimicrobial activity and the ability to enhance antimicrobial activities pre-existing in the human blood environment, present a very attractive therapeutic option either as stand-alone agents or in combination with antibiotics.

Considering the clinical plan for systemic use of PlySs2 (CF-301), human blood is the most relevant testing matrix for PlySs2 (CF-301) activity. In the current investigation, we used ex vivo screening of lysin PlySs2 (CF-301) activity against a range of S. aureus isolates in the context of human blood matrices. The anti-staphylococcal activity of PlySs2 (CF-301) both as a single agent and in combinations with antibiotics was found to be consistently more effective in blood matrices as compared to artificial media. Furthermore, human lysozyme and serum albumin each demonstrated a strong synergistic effect with PlySs2 (CF-301), and accounted for much of the observed blood enhancer effect in vitro. As an in vivo proof of concept the efficacy of PlySs2 (CF-301) was tested in an infective endocarditis model using both rabbits (with a blood effect equivalent to humans) and rats (with an intermediate blood effect). The in vivo studies demonstrated a ˜50 fold higher dose required for a bactericidal effect in rats compare to rabbits. Overall, our results demonstrate that the antimicrobial activity of PlySs2 (CF-301) is enhanced in human blood by virtue of synergy with at least two blood components. The ability of PlySs2 (CF-301) to synergize with blood factors and with antibiotics in a complex human fluid represents a very important attribute for a novel antimicrobial now under clinical development.

### Results

Time-Dependent Killing in Human Blood Matrices

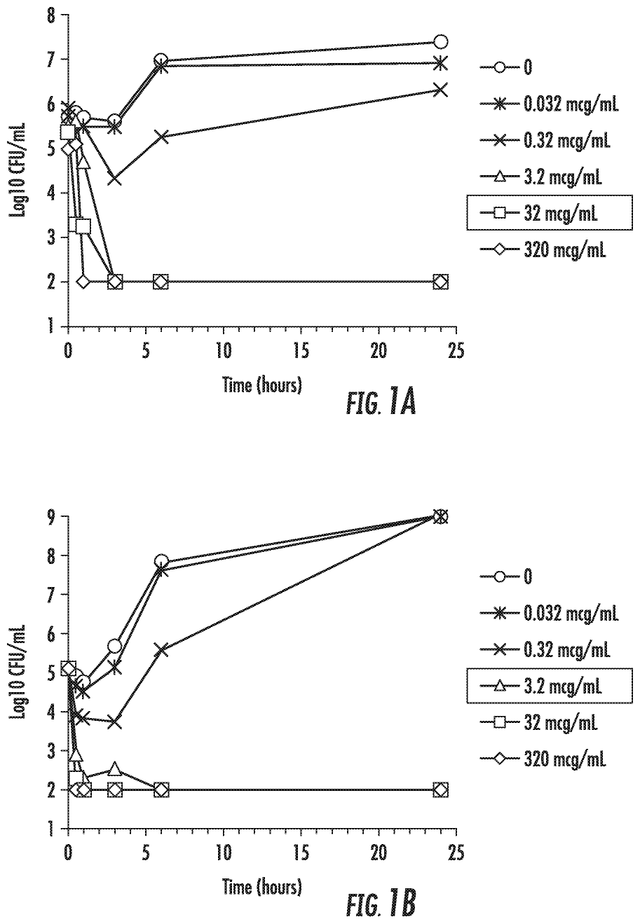

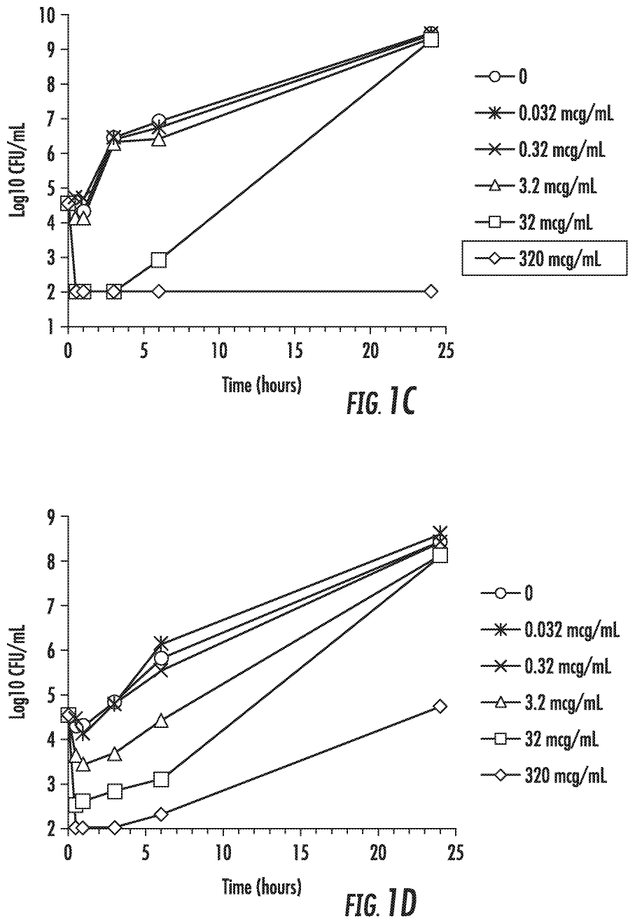

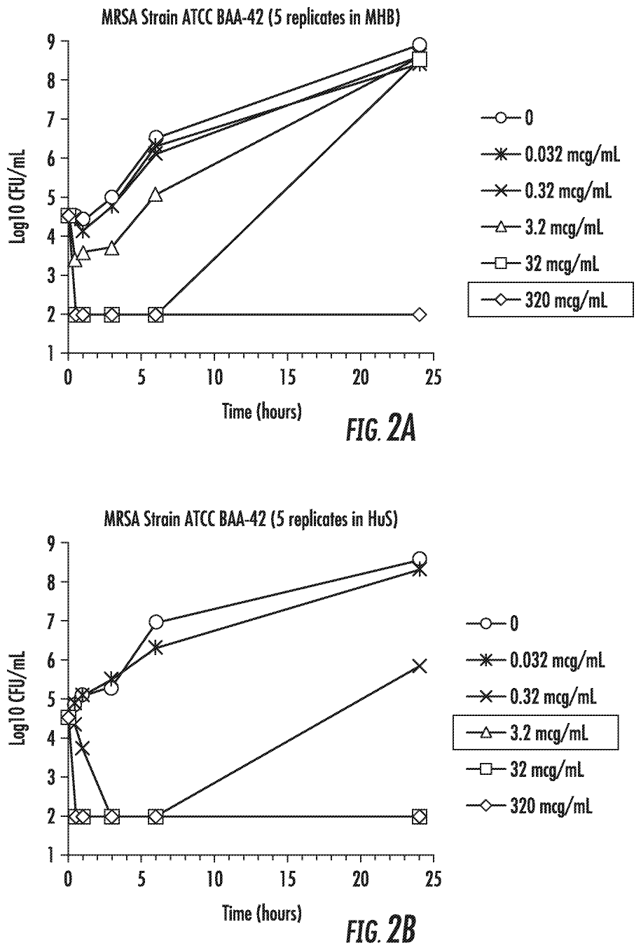

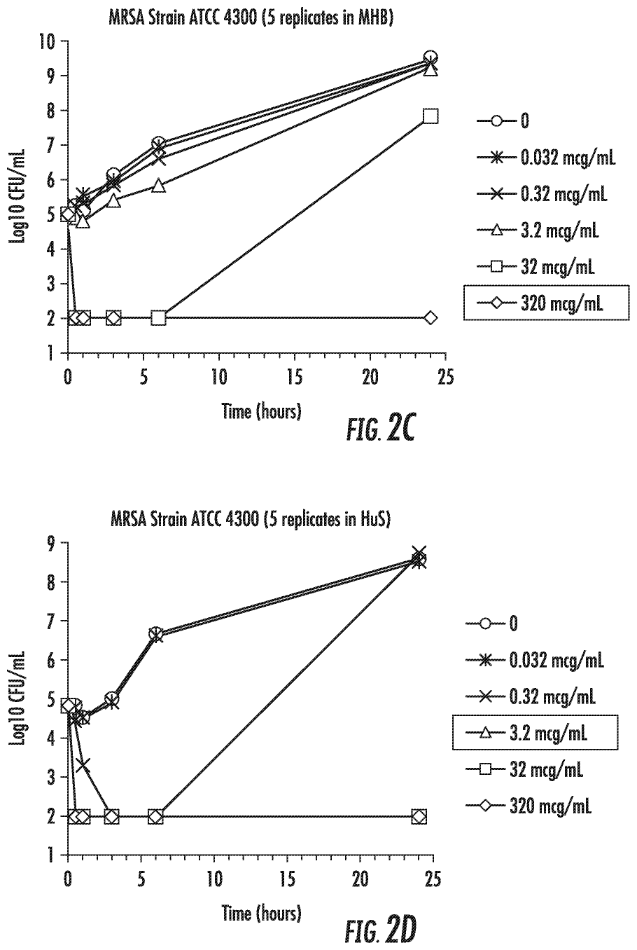

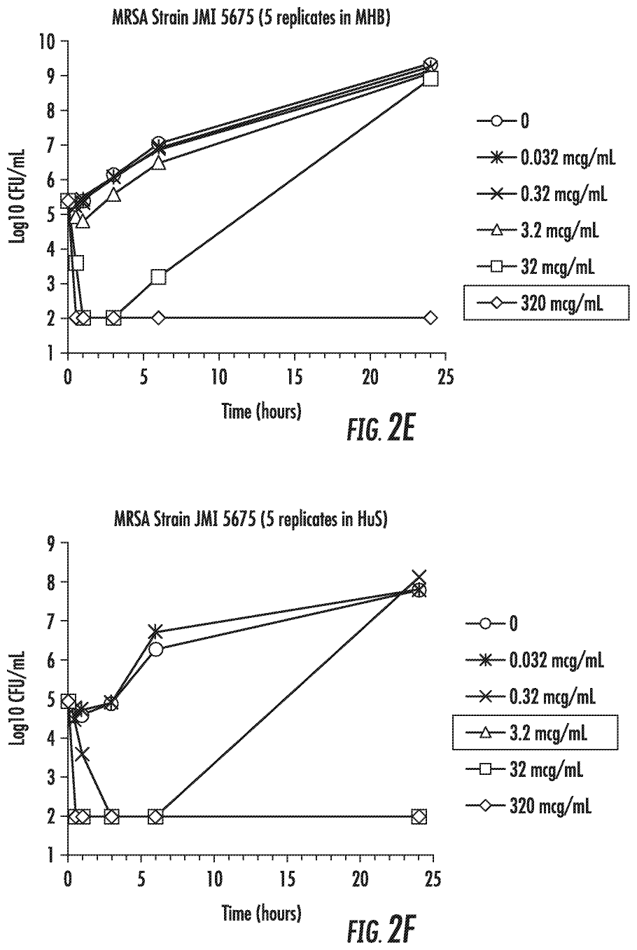

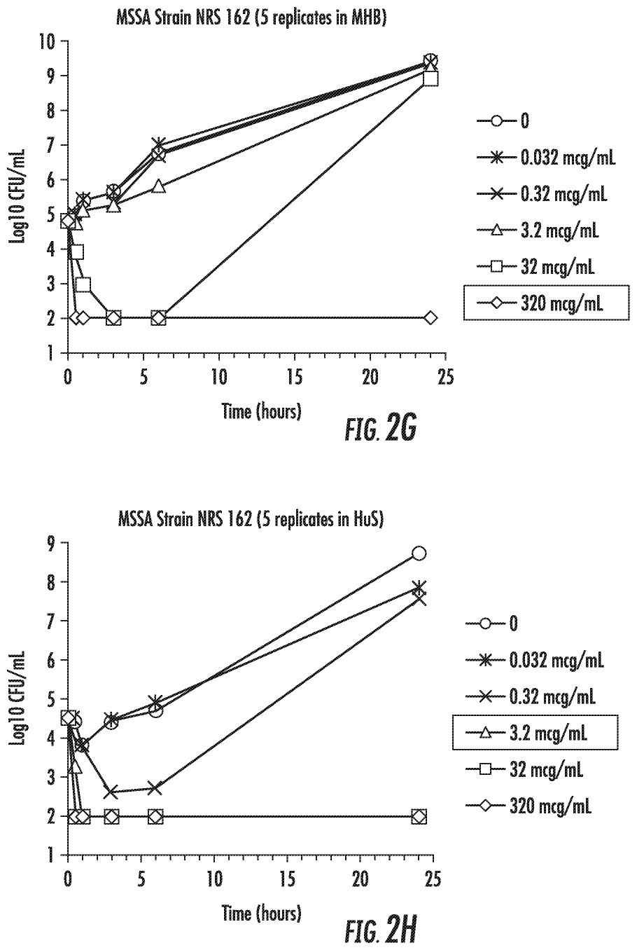

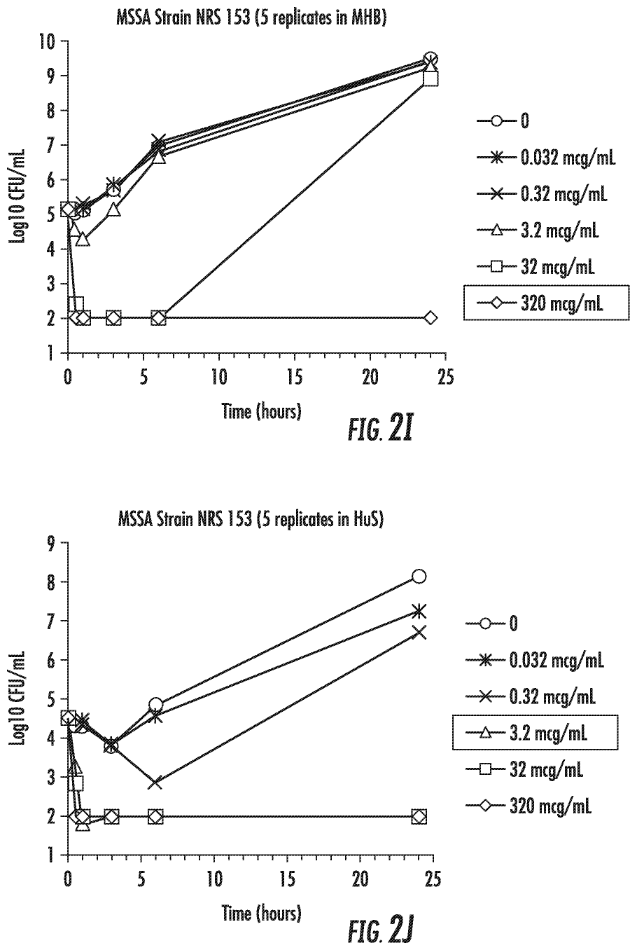

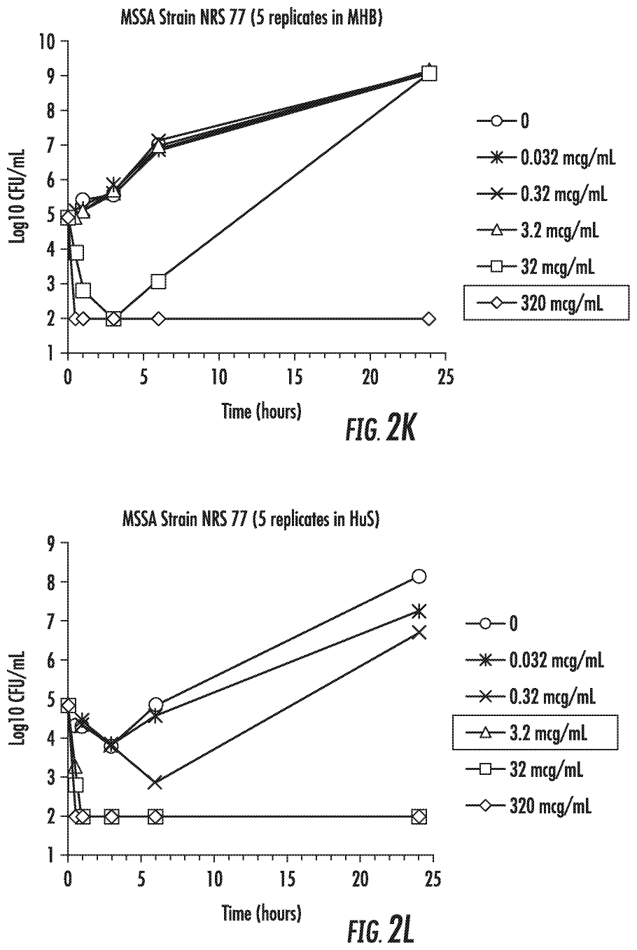

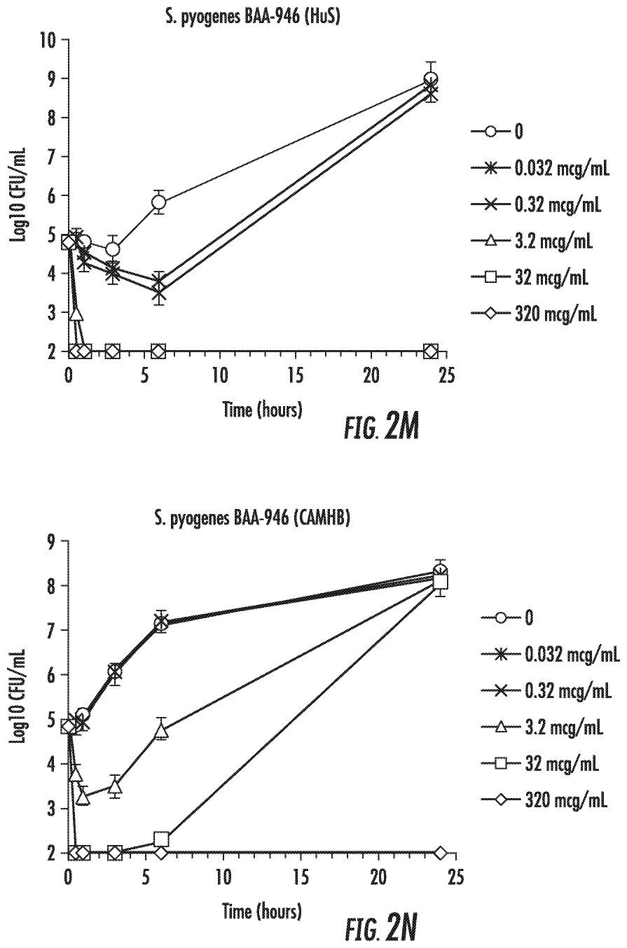

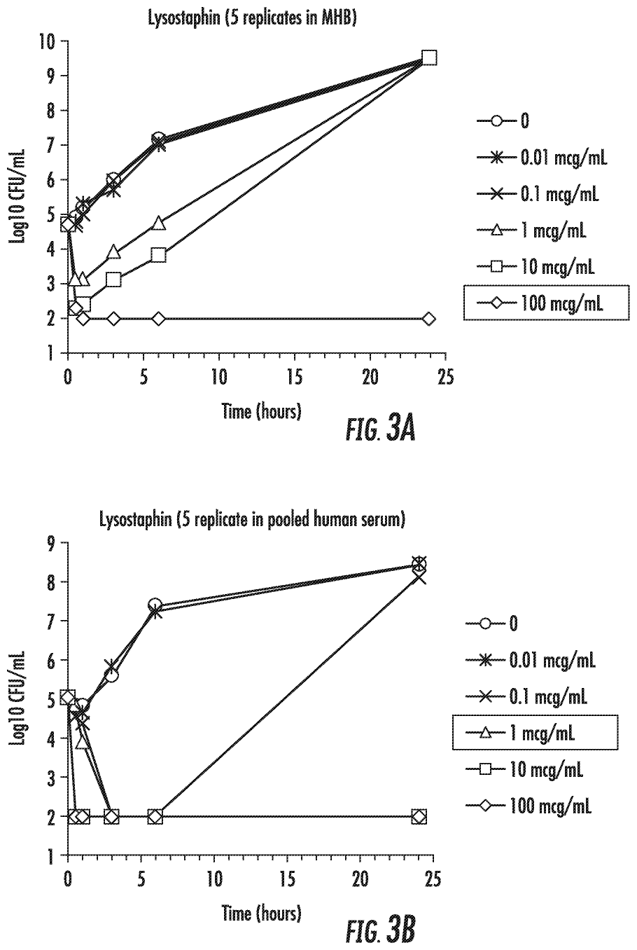

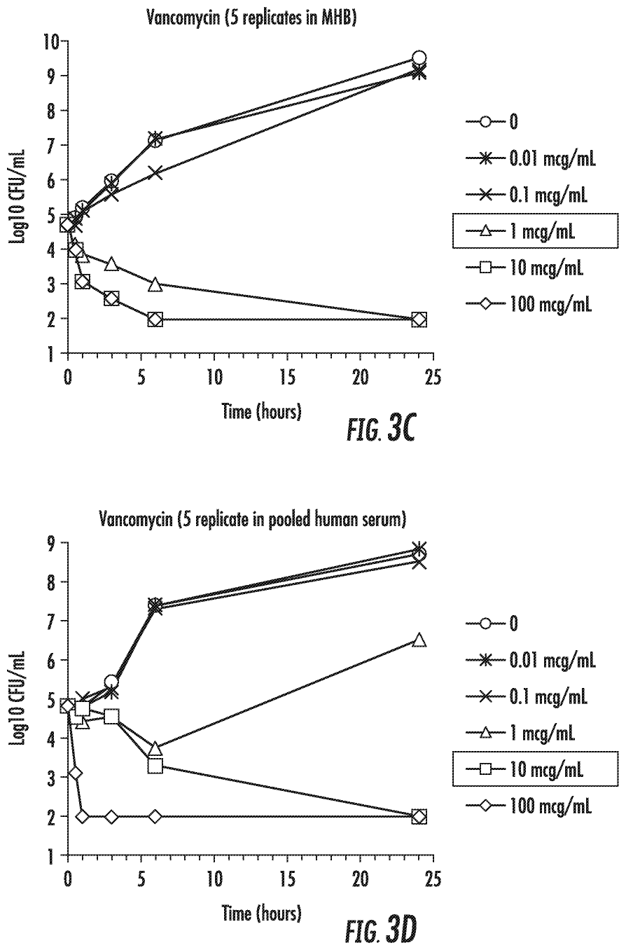

Time-kill assays were used to assess the time-dependent bactericidal activity of PlySs2 (CF-301), over a range of concentrations, against methicillin-resistant S. aureus (MRSA) strain MW2. A variation of the methodology published by the Clinical and Laboratory Standards Institute (CLSI) (CLSI document M07-A9 (Methods for dilutional antimicrobial sensitivity tests for bacteria that grow aerobically. Volume 32 (Wayne [PA]: Clinical and Laboratory Standards Institute [US], 2012)) was used whereby the standard testing medium (i.e., Mueller Hinton broth [MHB]) was replaced with human whole (heparinized) blood, serum, or plasma. In composite time-kill curves, PlySs2 (CF-301) was rapidly bactericidal (≥3-log 10 CFU/mL reduction) and sterilizing (by 24 hours post-treatment) at concentrations down to 3.2 μg/mL in all human blood matrices (FIG. 1A, 1B, and data not shown). In contrast, the minimum sterilizing concentration of PlySs2 (CF-301) in MHB was 320 μg/mL (FIG. 1C). The 100-fold difference in sterilizing activity observed for MW2 was similarly observed for 3 additional MRSA strains, 3 methicillin-sensitive S. aureus strains (MSSA) and 1 Streptococcus pyogenes strain (FIG. 2). In addition to PlySs2 (CF-301), a second lysin-like enzyme, lysostaphin (20), was also tested and, as with PlySs2 (CF-301), demonstrated a 100-fold decrease in the concentration required to sterilize in blood compared to MHB (FIGS. 3A and 3B). As a control, vancomycin was also examined and demonstrated a slightly decreased potency in blood than in MHB (FIGS. 3C and 3D).

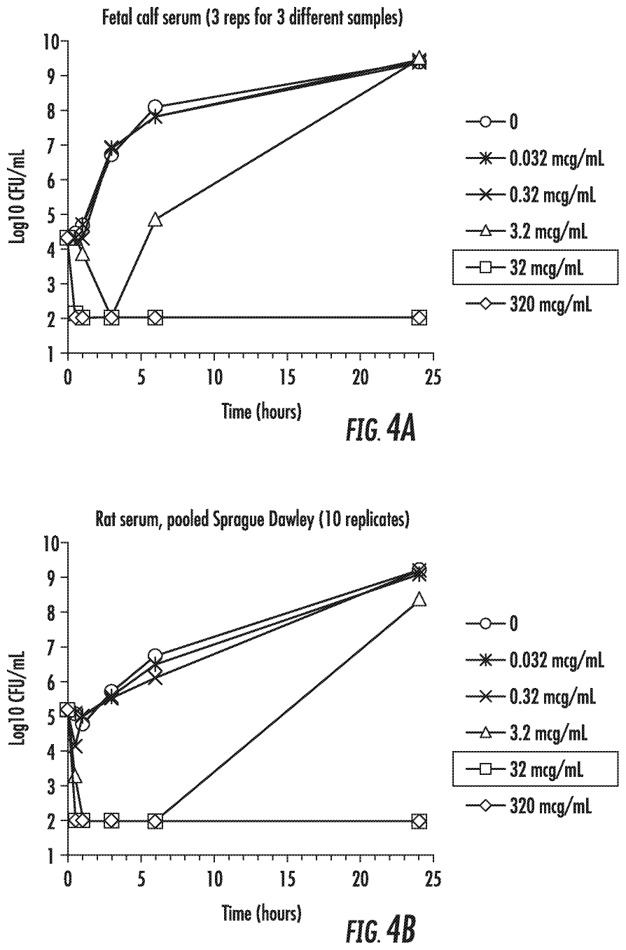

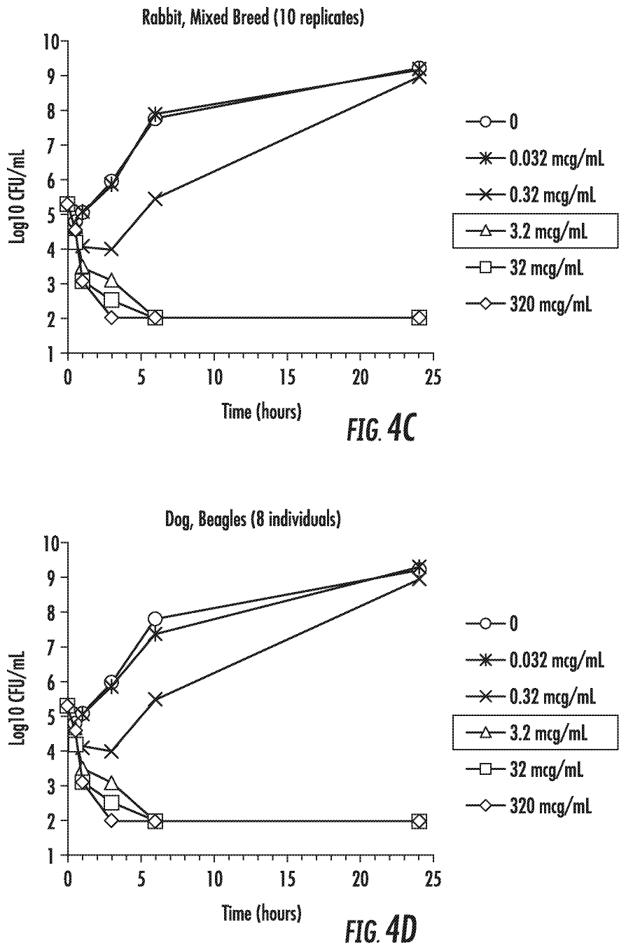

Time-kill experiments were also performed with PlySs2 (CF-301) to examine the extent of the blood effect in different species (in comparison to human matrices and MHB). The activity of PlySs2 (CF-301) in mouse serum was most similar to that in MHB, with a sterilizing concentration of >320 μg/mL (FIG. 1D). In calf and rat serum, an intermediate effect was observed whereby the sterilizing concentration was 32 μg/mL (FIGS. 4A and 4B). In rabbit and dog serum a human-like blood effect was observed with a sterilizing concentration of 3.2 μg/mL (FIGS. 4C and 4D). The time-kill results are consistent with the following hierarchy for blood-associated enhancement: human=rabbit=dog>rat=calf>MHB>mouse. Earlier studies has indicated that horse serum would substitute for human serum in time kill assays, particularly with an added reducing agent such as DTT. These are described in PCT US2017/32344 filed May 12, 2017 based on U.S. 62/335,129 filed May 12, 2016, incorporated herein by reference. Horse serum also demonstrates a similar enhancement, therefore human=rat=dog=horse.

Minimal Inhibitory Concentrations in Human Blood Matrices

The minimal inhibitory concentration (MIC) provides a quantitative measure of antimicrobial activity in a static system at a fixed 18 hour endpoint. The CLSI method for determining MICs by broth-microdilution was used to evaluate PlySs2 (CF-301) activity in either the standard testing medium (i.e., MHB) or human serum against a range of 171 clinical S. aureus isolates. PlySs2 (CF-301) demonstrated enhanced potency in human serum compared to MHB for all strains tested, including 74 MSSA, 75 MSSA, and additional vancomycin-resistant, linezolid-resistant and daptomycin-resistant strains (TABLE 1). Overall, there was a 32-fold decrease in the PlySs2 (CF-301) concentration needed to inhibit growth for 90% of the isolates tested (MIC₉₀) in each group. Interestingly, both the anti-staphylococcal lysin Sal1 and the lysin-like protein lysostaphin also exhibited pronounced 32-fold decrease in MICs when tested in blood matrices (TABLE 2). Lysin ClyS, however, demonstrated only a modest 2-fold shift.

TABLE 1
Comparison of MIC values obtained
using CAMHB and human serum

| S. aureus | | CAMHB | | | Human serum | | |
|---|---|---|---|---|---|---|---|
| type | N | MIC₅₀ | MIC₉₀ | Range | MIC₅₀ | MIC₉₀ | Range |
| MSSA | 74 | 16 | 32 | 8-32 | 0.5 | 1 | 0.25-1 |
| MRSA | 75 | 32 | 32 | 2-128 | 0.5 | 1 | 0.25-2 |
| Other* | 22 | 4 | 32 | 0.5-32 | 0.5 | 1 | 0.25-2 |

*S. aureus types tested include 12 vancomycin-resistant strains, 5 linezolid-resistant strains, and 5 daptomycin-resistant strains.

TABLE 2
MIC Analysis of Various Lysins and
Antibiotics in MHB and Human Serum

| | Minimal inhibitory | | |
|---|---|---|---|
| | concentration (μg/mL) | | |
| | | Human | Fold decrease |
| Agent | MHB | serum | in MIC |
| CF-301 | 32 | 1 | 32 |
| Lysostaphin | 2 | 0.0039 | 512 |
| Sal1 | 2 | 0.06 | 32 |
| ClyS | 8 | 4 | 2 |
| Daptomycin | 0.5 | 8 | no |
| Vancomycin | 1 | 1 | no |

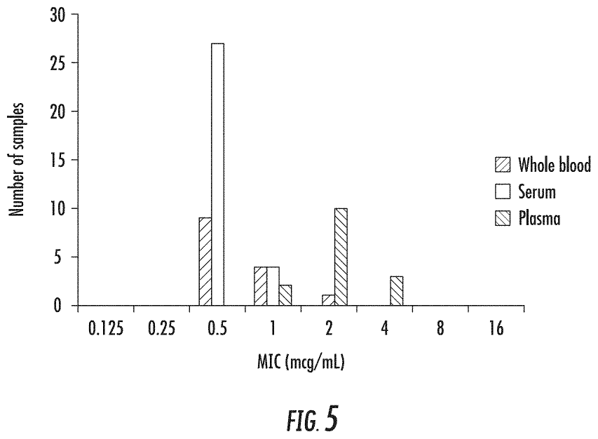

To understand whether variation in the sources of human blood-derived matrices may impact activity, PlySs2 (CF-301) MICs were determined using an array of 62 different pooled and individual human donor samples of whole blood, serum and plasma from different commercial sources with variations in age, sex, blood type, and type of anticoagulant used. For the whole blood (n=13), serum (n=33), and plasma (n=15) samples tested, a PlySs2 (CF-301) MIC₉₀ of value of 1 μg/mL was observed with a range of 0.5-2 μg/mL (TABLE 3). Histograms showing MIC frequencies for each of the different matrices indicate that activity in whole blood and serum is equivalent, while activity in plasma differs by ˜1-log 2 dilution (FIG. 5). Overall, the enhancer effect was not impacted by variations in the age, sex, and blood type of individual or pooled donors or by the use of sodium citrate or sodium heparin as anticoagulants (TABLE 3). The effect was also observed in complement-inactivate media, complement preserved media, and was equivalent in at least 3 different matched sets of human blood, serum and plasma fractions, while delipidated serum was not equivalent.

TABLE 3

| | | | | MIC |
|---|---|---|---|---|
| Blood matrix¹ | Vendor | Source description | Lot Number | (mg/mL)² |
| Whole blood (heparin) | Bioreclamation | Individual, Male, Black, 29 yo | BRH1025972 | 0.5 |
| Whole blood (heparin) | Bioreclamation | Individual, Male, Hispanic, 35 yo | BRH1025973 | 1 |
| Whole blood (citrate) | Bioreclamation | Individual, Male, Black, 52 yo | BRH1149915 | 0.5 |
| Whole blood (heparin) | Bioreclamation | Individual, Male, Hispanic, 30 yo | BRH1085022 | 1 |
| Whole blood, (heparin) | Bioreclamation | Individual, Male, Hispanic, 56 yo | BRH1085021 | 0.5 |
| Whole blood (citrate) | Research Blood | Individual, Male, Black, Type O+ | KP34988 | 0.5 |
| | Components | | | |
| Whole blood (citrate) | Research Blood | Individual, Male, Hispanic, Type A+ | KP35077 | 0.5 |
| | Components | | | |
| Whole blood (citrate) | Research Blood | Individual, Male, Hispanic, Type O+ | KP35332 | 0.5 |
| | Components | | | |
| Whole blood (citrate) | Research Blood | Individual, Female, Caucasian, Type A+ | KP33586 | 0.5 |
| | Components | | | |
| Whole blood (citrate) | Research Blood | Individual | KP35818 | 1 |
| | Components | | | |
| Whole blood (citrate) | Research Blood | Individual | KP35734 | 0.5 |
| | Components | | | |
| Whole blood (citrate) | Research Blood | Individual | KP35581 | 1 |
| | Components | | | |
| Whole blood (citrate) | Research Blood | Individual | KP30156 | 0.5 |
| | Components | | | |
| Serum | Research Blood | Individual, Male, Black, Type O+ | KP35534 | 0.5 |
| | Components | | | |
| Seram | Research Blood | Individual, Male, Caucasian, Type O− | KP35567 | 0.5 |
| | Components | | | |
| Serum | Innovation | Pooled, Male, Type AB, from plasma | IPLA-SERAB- | |
| | Research | | 19799 | |
| Serum | Sigma-Aldrich | Pooled, Male, Type AB, from plasma | SLBG7011V | 0.5 |
| Serum | Sigma-Aldrich | Pooled, Male, Type AB, from plasma | SLBC8760V | 0.5 |
| Serum | Sigma-Aldrich | Pooled, Male, Type AB, from plasma | SLBJ3902V | 0.5 |
| Serum | Sigma-Aldrich | Pooled, Male, Type AB, from plasma | SLBG2954V | 0.5 |
| Serum | Sigma-Aldrich | Pooled, Male, Type AB, from plasma | SLBL0334V | 0.5 |
| Serum | Sigma-Aldrich | Pooled, Male, Type AB, from plasma | SLBK7465V | 0.5 |
| Serum | Sigma-Aldrich | Pooled, Male, Type AB, from plasma | SLBM3312V | 0.5 |
| Serum | Sigma-Aldrich | Pooled, Male, Type AB, from plasma | SLBN4663V | 0.5 |
| Serum | Sigma-Aldrich | Pooled, Male, Type AB, from plasma | SLBP6097V | 0.5 |
| Serum | Sigma-Aldrich | Pooled, Male, Type AB, from plasma | SLBN4664V | 0.5 |
| Serum | Sigma-Aldrich | Pooled, Male, Type AB, from plasma | SLBP7640V | 0.5 |
| Serum | Sigma-Aldrich | Pooled, Male, Type AB, from plasma | SLPQ9160V | 0.5 |
| Serum | Sigma-Aldrich | Pooled, Male, Type AB, from plasma | SLBQ3969V | 0.5 |
| Serum | Research Blood | Individual, Female, Hispanic, Type B+ | KP35523 | 0.5 |
| | Components | | | |
| Serum | Research Blood | Individual, Female, Hispanic, Type O+ | KP35546 | 1 |
| | Components | | | |
| Serum | Research Blood | Individual, Male, Black, Type O+ | KP35547 | 0.5 |
| | Components | | | |
| Serum | Research Blood | Individual, Male, Caucasian, Type A+ | KP35568 | 1 |
| | Components | | | |
| Serum | Research Blood | Individual, Male, Caucasian, Type A+ | KP35569 | 0.5 |
| | Components | | | |
| Serum | Research Blood | Individual, Female, Black, Type A+ | KP35601 | 0.5 |
| | Components | | | |
| Serum | Research Blood | Individual, Female, Caucasian, Type A+ | KP35603 | 0.5 |
| | Components | | | |
| Serum | Research Blood | Individual | KP35617 | 0.5 |
| | Components | | | |
| Serum | Bioreclamation | Individual, Male, Caucasian, 57 yo | BRH1297980 | 0.5 |
| Serum | Bioreclamation | Individual, Male, Caucasian, 58 yo | BRH1297981 | 0.5 |
| Serum | Bioreclamation | Individual, Female, Black, 39 yo | BRH1297982 | 0.5 |
| Serum, heat-inactivated | Bioreclamation | Individual, Male, Caucasian, 55 yo | BRH1297983 | 0.5 |
| Serum, heat-inactivated | Bioreclamation | Individual, Female, Black, 34 yo | BRH1297984 | 0.5 |
| Serum, heat-inactivated | Bioreclamation | Individual, Female, Black, 41 yo | BRH1297985 | 1 |
| Serum, complement | Bioreclamation | Individual, Male, Black, 60 yo | BRH1297986 | 1 |
| preserved | | | | |
| Serum, complement | Bioreclamation | Individual, Male, Black, 50 yo | BRH1297987 | 2 |
| preserved | | | | |
| Serum, complement | Bioreclamation | Individual, Male, Black, 43 yo | BRH1297988 | 0.5 |
| preserved | | | | |
| Serum, delipidized | Innovative | Pooled, derived from plasma | IPLA-SER- | 64 |
| | Research | | AHBS | |
| Serum, delipidized | Bioreclamation | Pooled, derived from plasma | BFH1377020 | 64 |
| Serum, delipidized | Bioreclamation | Pooled, derived from plasma | BFH1377021 | 64 |
| Serum, delipidized | Valley Biomedical | Pooled, derived from plasma | 5L2186 | 64 |
| Plasma (citrate) | Bioreclamation | Pooled | BRH1125647 | 1 |
| Plasma (citrate) | Bioreclamation | Pooled | BRH1215896 | 2 |
| Plasma (citrate) | Bioreclamation | Pooled | BRH1149506 | 2 |
| Plasma (citrate) | Bioreclamation | Pooled | BRH1149505 | 2 |
| Plasma (citrate) | Bioreclamation | Male, Black, 25 yo | BRH1292304 | 2 |
| Plasma (citrate) | Bioreclamation | Male, Black, 62 yo | BRH1292305 | 2 |
| Plasma (citrate) | Bioreclamation | Female, Black, 32 yo | BRH1292306 | 2 |
| Plasma (citrate) | Bioreclamation | Male, Caucasian, 45 yo | BRH1292307 | 2 |
| Plasma (citrate) | Bioreclamation | Female, Hispanic, 38 yo | BRH1292308 | 2 |
| Plasma (K₂ EDTA) | Bioreclamation | Pooled | BRH1271729 | 2 |
| Plasma (K₂ EDTA) | Bioreclamation | Male, Black, 36 yo | BRH1245950 | 2 |
| Plasma (K₂ EDTA) | Bioreclamation | Male, Hispanic, 31 yo | BRH1245951 | 2 |
| Plasma (K₂ EDTA) | Bioreclamation | Male, Black, 26 yo | BRH1245952 | 1 |
| Plasma (K₂ EDTA) | Bioreclamation | Female, Black, 33 yo | BRH1245953 | 2 |
| Plasma (K₂ EDTA) | Bioreclamation | Female, Caucasian, 55 yo | BRH1245954 | 2 |

¹The anticoagulant is shown in parentheses.
²The MIC was determined by broth microdilution in triplicate on consecutive days.

The impact of sample variation on PlySs2 (CF-301) activity in the serum of different animal species was also examined. The MIC range for mouse samples (n=10) was 32-64 ug/mL, while the range for rat (n=5) was 8-16 ug/ml, the range for dog (n=8) was 0.5-1 ug/ml and rabbit (n=2) was 1 μg/mL (TABLE 4). The hierarchy observed here, rabbit and dog as superior to rat and mouse is identical to that observed in the time kill studies above.

TABLE 4
PlySs2 (CF-301) MIC values for S. aureus strain
MW2 in serum from a range of animal species

| | | | | PlySs2/CF-301 |
|---|---|---|---|---|
| Blood matrix | Vendor | Source description | Lot number | MIC (μg/mL) |
| Serum, mouse | Bioreclamation | Pooled, CD-1 | MSE217937 | 64 |
| Serum, mouse | Bioreclamation | Pooled, CF-1 | MSE218431 | 32 |
| Serum, mouse | Bioreclamation | Pooled, Black Swiss | MSE218429 | 32 |
| Serum, mouse | Bioreclamation | Pooled, ICR | MSE217934 | 32 |
| Serum, mouse | Bioreclamation | Pooled, ICR | MSE217935 | 32 |
| Serum, mouse | Bioreclamation | Pooled, ICR | MSE217936 | 32 |
| Serum, mouse | Bioreclamation | Pooled, BALB/c | MSE217938 | 64 |
| Serum, mouse | Bioreclamation | Pooled, BALB/c | MSE217939 | 64 |
| Serum, mouse | Bioreclamation | Pooled, BALB/c | MSE232989 | 64 |
| Serum, mouse | Bioreclamation | Pooled, BALB/c | MSE217940 | 64 |
| Serum, dog | Biochemed | Individual male, Beagle | S130048 | 1 |
| Serum, dog | Biochemed | Individual male, Beagle | S130047 | 1 |
| Serum, dog | ABCAM | Pooled, Beagle | GR178396 | 0.5 |
| Serum, dog | Ridglan Farms | individual, Beagle | WPH3 | 0.5 |
| Serum, dog | Ridglan Farms | Individual, Beagle | FJV2 | 0.5 |
| Serum, dog | Lampire | individual male, Beagle | 14F21032 | 0.5 |
| Serum, dog | Lampire | Individual female, Beagle | 14F21031 | 1 |
| Serum, dog | Innovative Research | Pooled, Beagle | 15173 | 0.5 |
| Serum, rabbit | Gibco | Mixed breed | 1435334 | 1 |
| Serum, rabbit | Gibco | Mixed breed | 1723604 | 1 |
| Serum, rat | Bioreclamation | Pooled, Sprague-Dawley | RAT239345 | 8 |
| Serum, rat | Bioreclamation | Pooled, Sprague-Dawley | n.a. | 8 |
| Serum, rat | Bioreclamation | Pooled, Sprague-Dawley | n.a. | 16 |
| Serum, rat | Sigma | Pooled | n.a. | 16 |
| Serum, rat | Sigma | Pooled | n.a. | 16 |

All MIC values were determined by broth microdilution in triplicate on two consecutive days using the indicated blood matrix (undiluted) as the growth medium.

PlySs2 (CF-301) Synergizes with Antibiotics in Human Blood Matrices

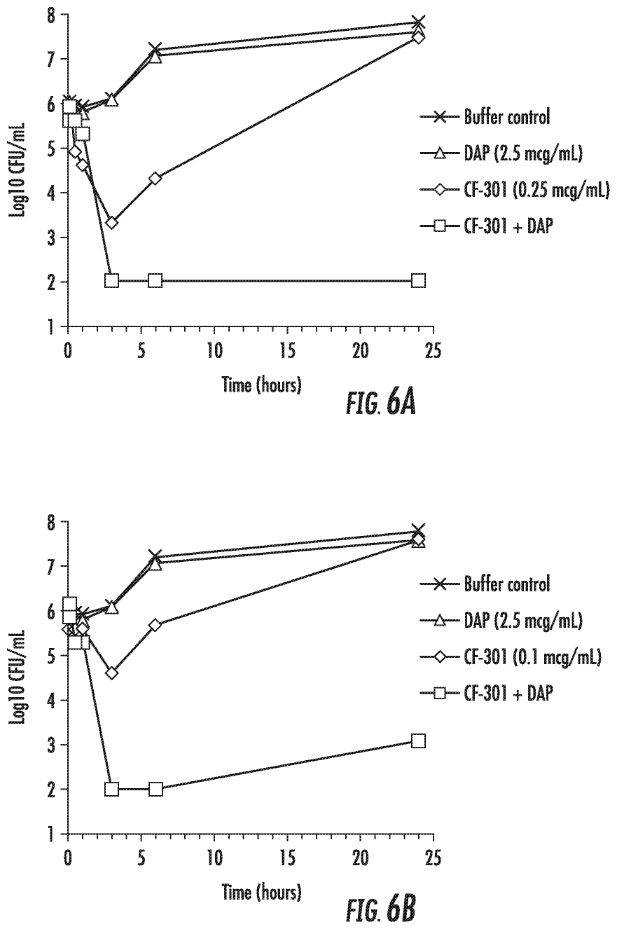

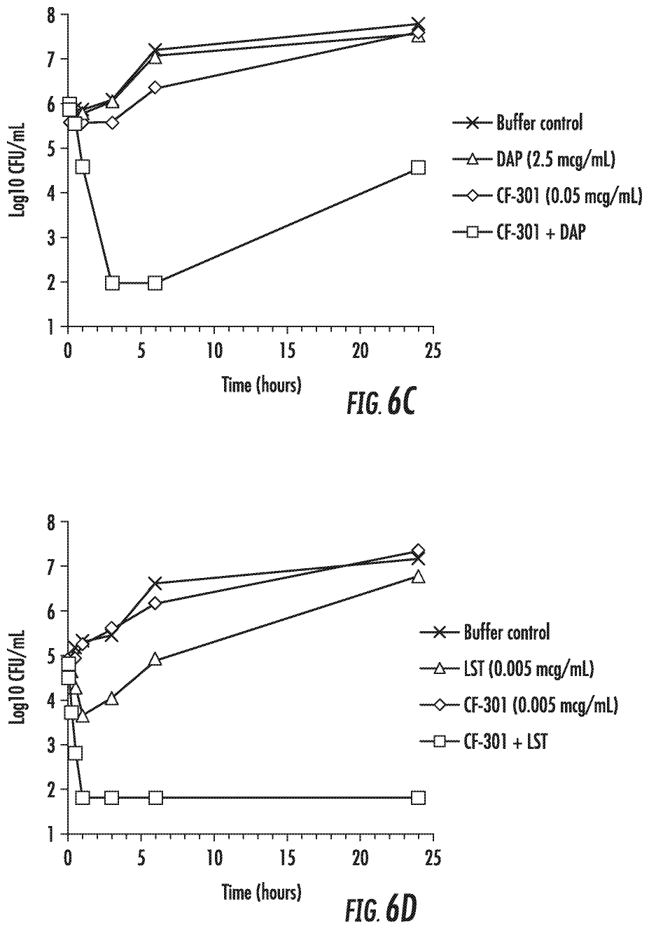

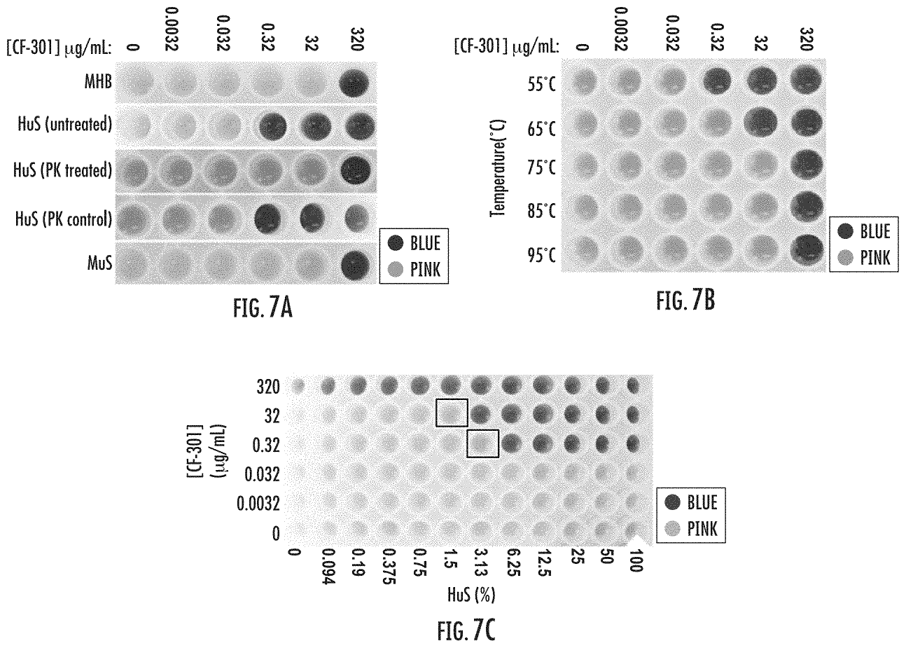

Synergy between PlySs2 (CF-301) and either lysostaphin, daptomycin or vancomycin in the context of human serum was assessed using 2 different methods. The first method was the time-kill assay, a preferred technique for examining synergistic antimicrobial activity in vitro. Sub-MIC amounts of daptomycin and PlySs2 (CF-301) were tested individually and found to have a minimal bactericidal effect on S. aureus strain MW2 (FIG. 6A-6C). However, when the same amounts of each agent were combined, substantially more killing was observed (?2-log 10 CFU/mL reductions), consistent with synergy. Highly synergistic interactions were similarly observed for combinations of sub-MIC PlySs2 (CF-301) with either lysostaphin (FIG. 6D) or vancomycin (data not shown).

In the time-kill format, PlySs2 (CF-301) exhibited synergy across a range of sub-MIC concentrations (0.25-0.025 ug/mL) with a constant amount of DAP (2.5 ug/mL). Synergy is defined as a ≥2-log 10 decrease in CFU/mL at a 24-hour time-point. This represents a 64-fold decrease in the minimum sterilizing concentration of PlySs2 (CF-301) (as a single agent) required in the time-kill assay performed in human serum. Similarly, potent synergy was observed in the time-kill between PlySs2 (CF-301) and another antimicrobial agent, lysostaphin. Here, combinations of each agent (at 0.005 mcg/mL) are sterilizing at 24 hours. Of note, the minimum concentration of PlySs2 (CF-301) demonstrating synergy with daptomycin in human serum (i.e., 0.025 μg/mL) was 160× lower than the minimum concentration of 4 μg/mL required for synergy with daptomycin in CAMHB (Schuch R. et al (2014) J Infect Dis 209(9):1469-1478), suggesting the potential link between host factors in human blood and the bactericidal activity of PlySs2 (CF-301).

A second method to confirm synergy was the checkerboard assay (Verma P (2007) Methods for Determining Bactericidal Activity and Antimicrobial Interactions: Synergy Testing, Time-Kill Curves, and Population Analysis. Antimicrobial Susceptibility Testing Protocols, eds Schwalbe R, Steele-Moore L, & Goodwin A C (CRC Press, Boca Raton, Fla.), pp 275-298). Checkerboards were generated using combinations of sub-MIC PlySs2 (CF-301) with either sub-MIC lysostaphin, daptomycin or vancomycin against MRSA strain MW2 in either MHB or human serum. Synergy was defined as inhibitory activity great than what would be predicted by adding the 2 drugs together (i.e., minimum or average fractional inhibitory concentrations [FIC_{MIN} or FIC_{AVG}]≤0.5). PlySs2 (CF-301) demonstrated very potent synergy with lysostaphin, daptomycin and vancomycin in human serum compared to MHB (TABLE 5). The reduced FIC values even more significant, considering the 32× difference in MIC values for PlySs2 (CF-301) in serum compared to MHB. In an extended analysis of 8 additional MRSA strain, the FIC_{MIN} and FIC_{AVG} values were consistently below 0.5 (i.e., synergy) and superior to that determined in MHB (TABLE 6).

TABLE 5

| Antimicrobial | HuS | | MHB | |
|---|---|---|---|---|
| agent | ΣFIC_{MIN}^{a} | ΣFIC_{AVG}^{b} | ΣFIC_{MIN} | ΣFIC_{AVG} |
| lysostaphin | 0.09 | 0.13 | 0.275 | 0.5 |
| daptomycin | 0.25 | 0.292 | 0.5 | 0.63 |
| vancomycin | 0.375 | 0.5 | 0.5 | 0.63 |

^{a}ΣFIC_{MIN} = summation of fractional inhibitory concentrations (lowest value observed among all combinations).
^{b}ΣFIC_{AVG} = summation of fractional inhibitory concentrations (average of three consecution combinatons with the lowest values).

TABLE 6

| | |
|---|---|
| Strain | CAMHB |

| Number | ΣFICₘᵢₙ | ΣFIC_{avg} | ΣFICₘᵢₙ | ΣFIC_{avg} |
|---|---|---|---|---|
| NRS 271 | 0.25 | 0.39 | 0.38 | 0.5 |
| NRS 100 | 0.25 | 0.29 | 0.5 | 0.75 |
| ATCC 43300 | 0.25 | 0.29 | 0.5 | 0.87 |
| HPV 107 | 0.38 | 0.44 | 0.5 | 0.64 |
| CAIRD 456 | 0.38 | 0.44 | 0.5 | 0.75 |
| JMI 227 | 0.25 | 0.29 | 0.5 | 0.64 |
| JMI 1280 | 0.25 | 0.29 | 0.5 | 0.57 |
| JMI 4789 | 0.25 | 0.29 | 0.5 | 0.64 |

ΣFICₘᵢₙ is the lowest summation value observed among all combinations of each paired agent.
ΣFIC_{avg} is the average summation value for at least three consecutive combinations of each paired agent.

PlySs2 (CF-301) Synergizes with Human Lysozyme and Human Serum Albumin

To understand the basis of the human blood effect, a series of assays were used to examine certain physical features of the enhancer agent/s. A colorimetric based MIC assays was used to demonstrate that the enhancer effect of human serum was both sensitive to proteinase K (FIG. 7A) and was completely inactivated at temperatures above 75° C. (FIG. 7B). Furthermore, the enhancer effect was diluted out at serum concentrations of 6.25-1.5% (FIG. 7C). These finding are consistent with a proteinaceous enhancer agent/s that is both heat stable and abundant. Based both on these findings, and on literature describing the potential for antibiotics and AMPs to synergize with host blood components, we next tested the anti-staphylococcal activity of a range of potentially antibacterial blood proteins in combination with PlySs2 (CF-301) in the checkerboard assay using heat-inactivated human serum as the base medium (TABLE 7). While PlySs2 (CF-301) did not synergize with the majority of agents tested (based on FIC_{AVG} values of >0.5), notable synergistic interactions were detected with both the purified and recombinantly expressed forms of either human lysozyme (HuLYZ) or human serum albumin (HSA). FIC_{AVG} values of ≤0.1 were observed, consistent with very strong synergy in serum. Significantly, rabbit serum albumin (RabSA) synergized with PlySs2 (CF-301) (FIC_{AVG}≤0.1), while rat serum albumin (RatSA) and mouse serum albumin (MSA) did not (FIC_{AVG} >1.16).

TABLE 7
Checkerboard Analysis of PlySs2/CF-301 with AMPs, antimicrobial
proteins and albumins in heat-inactivated human serum

| Agent | Description | ΣFIC_{avg}^{a} |
|---|---|---|
| β-Defensin 3 | Human AMP (hBD-3) | 1 |
| LEAP-1 | Human AMP (Hepcidin) | 0.75 |
| LEAP-2 | Human AMP | 1 |
| LL-37 | Human AMP | 1 |
| Lactoferrin | Human milk | ≥1.16 |
| Lactoferrin | Bovine colostrum | ≥1.16 |
| Histatin-5 | Human AMP | ≥1.16 |
| HNP-1 | Human AMP | 1 |
| Lysozyme | Human, recombinant | ≤0.05 |
| Lysozyme | Hen egg-white | ≤0.563 |
| Lysozyme | Human neutrophil derived | ≤0.056 |
| Serum albumin | Human, fraction V | ≤0.086 |
| Serum albumin | Human, recombinant | ≤0.1 |
| Serum albumin | Mouse, from serum | ≥1.16 |
| Serum albumin | Mouse, recombinant | ≥1.16 |
| Serum albumin | Rat, from serum | ≥1.16 |
| Serum albumin | Rabbit, from serum | ≤0.1 |

^{a}ΣFIC_{AVG} = summation of fractional inhibitory concentrations (average of three consecutive combinations with the lowest values).

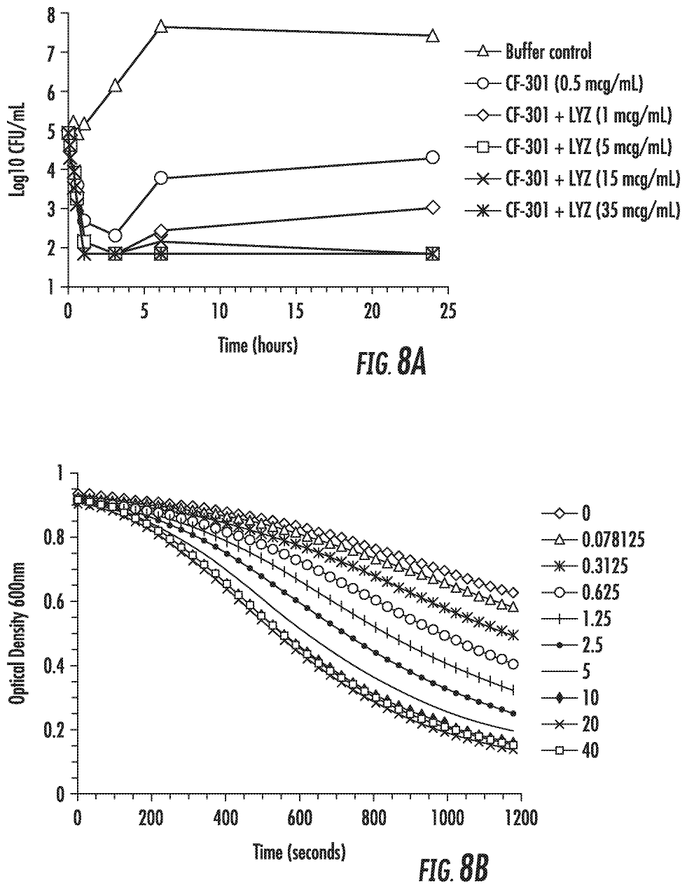

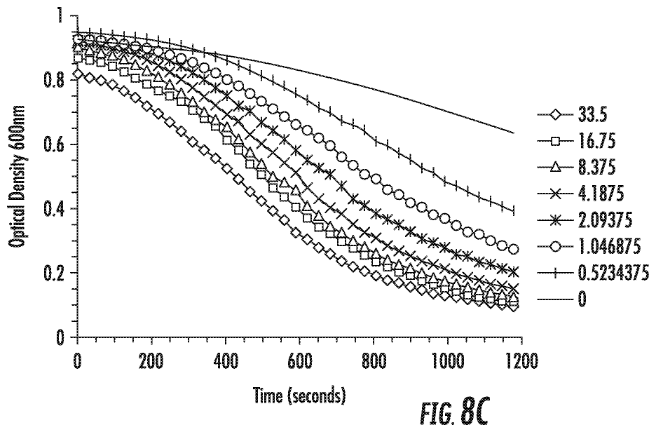

The effect of HuLYZ and HSA on PlySs2 (CF-301) activity was examined using additional formats. First, the time-kill assay was used to combine a sub-MIC amount of PlySs2 (CF-301) with a range of HuLYZ concentrations from 1-35 μg/mL (FIG. 8A). While HuLYZ alone has no anti-staphylococcal activity (21), the concentration in human blood is reported to vary between 1 and 35 μg/mL (22). At HuLYZ concentrations above 5 μg/mL a synergistic interaction was detected based on ≥2-log 10 CFU/mL reductions at 24 hours for the combinations compared to PlySs2 (CF-301) alone. In a second in vitro assay based on loss of optical density in a treated culture, the addition of both HuLYZ (FIG. 8B) and HSA (FIG. 8C) across a range of concentrations stimulated high-level PlySs2 (CF-301) activity. Human lysozyme alone was completely inactive against S. aureus at all concentrations tested. Similarly, HSA alone had no antibacterial activity in the absence of PlySs2 (CF-301). For HSA, which occurs in the blood at a concentration of ˜40 mg/mL (7), the maximal enhancement of PlySs2 (CF-301) activity was observed at HSA concentrations between 20 and 40 mg/mL. The lytic assay also serves as the basis for determining PlySs2 (CF-301) specific activity (4). While the activity of PlySs2 (CF-301) is standardly observed at ˜2500 Units/mg of protein, the addition of either HuLYZ or HSA results in a 9.8 or 17.8 fold increase in activity respectively (TABLE 8). If HuLYZ and HSA are added together, the fold increase in PlySs2 (CF-301) activity is 25%.

TABLE 8

| | Specific Activity | |
|---|---|---|
| Assay supplements | (Units/mg) | Fold increase |
| None | 2419 | n.a |
| rHSA, 4% | 43067 | 17.8 |
| HuLYZ, 10 μg/mL | 32066 | 9.8 |
| rHSA, HuLYZ | 18711 | 25 |

Assay performed using a fixed concentration of CF-301 (4 μg/mL) in phosphate buffer, with the indicated supplements, against S. aureus strain MW2.

Various commercially available lysozyme and HSA reagents were utilized and tested with similar results, as tabulated in TABLE 9 below.

TABLE 9

| Compound | Company | Catalog # | Source | |
|---|---|---|---|---|
| LYSOZYME | Aviva | OPMA04253 | Purified, | 95% pure, |
| | | | Human | lyophilized |
| | | | Neutrophils | from 0.05M |
| | | | | Sodium |
| | | | | Acetate, pH 6.0 |
| | | | | containing 0.1M |
| | | | | Sodium |
| | | | | Chloride |
| | RayBiotech | 227-10200 | Purified, | 95% pure, |
| | | | Human | lyophilized |
| | | | Neutrophils | from 0.05M |
| | | | | Sodium |
| | | | | Acetate, pH 6.0 |
| | | | | containing 0.1M |
| | | | | Sodium |
| | | | | Chloride |
| | RayBiotech | 227-10113 | Recombinant | Does not |
| | | | Human, from | indicate |
| | | | rice | |
| | Sigma | L1667-10G | Recombinant | >90% pure, |
| | | | Human, from | lyophilized |
| | | | rice | from 0.05 |
| | | | | Sodium acetate, |
| | | | | pH 6.0 |
| | | | | containing 0.1M |
| | | | | Sodium |
| | | | | Chloride |
| HSA | Albumin | 1001 | Recombinant | >98% pure |
| | Bioscience | | Human, from | |
| | | | yeast | |
| | Sigma | A9731-10G | Recombinant | >96% pure |
| | | | Human, from | |
| | | | rice | |
| | Sigma | A1653-10G | Fraction V, | 96-99% pure |
| | | | purified by | (remainder |
| | | | ethanol | mostly |
| | | | fractionation | globulins) |
| | Sigma | A3782-10MG | Prepared from | Fatty acid free, |
| | | | Fraction V | Globulin free, |
| | | | | >99% |
| | Sigma | A1887-10MG | Prepared from | Fatty acid free, |
| | | | Fraction V, heat | >96% purity |
| | | | step | |
| MSA | Albumin | 2601 | Recombinant | >95% pure |
| | Bioscience | | mouse serum | |
| | | | albumin from | |
| | | | yeast | |
| | Sigma | A3139-10MG | Prepared from | >96% pure |
| | | | Fraction V | |

Recapitulation of the Human Blood Effect Using HuLYZ and HSA

The basis of the human blood effect is a 32-fold decrease in MIC values determined in human serum compared to MHB. Using an MIC format, we sought to recapitulate the human blood effect by adding either HuLYZ and/or HSA to two different media types lacking a blood effect (i.e., MHB and MHB supplemented with 50% heat-inactivated human serum). The most potent lytic activity was observed with the combination of HuLYZ and HSA together with PlySs2 (CF-301). In MHB, the addition of either HuLYZ (at a concentration of 10 μg/mL) or HSA (at a concentration of 40 mg/mL) resulted in a 2-fold and 8-fold decrease in the PlySs2 (CF-301)MIC, respectively; the addition of both HuLYZ and HSA resulted in a 16-fold decrease (TABLE 10). The effect in MHB supplemented with 50% heat-inactivated human serum was similar (TABLE 11). Significantly, the addition of RabSA resulted in an effect similar to that observed for HSA (i.e, and 8-fold decrease), while the addition of either RatSA or MSA had little or no effect.

TABLE 10

| Supplementation of MHB | Fold decrease in MIC |
|---|---|
| HuLYZ, 10 μg/mL | 2 |
| HSA, 40 mg/mL | 8 |
| HuLYZ, 10 μg/mL + HSA 40 mg/mL | 16 |
| RabSA, 40 mg/mL | 8 |
| RatSA, 40 mg/mL | 2 |
| MouseSA, 40 mg/mL | 1 |

TABLE 11

| Supplementation of 50% MHB/50% | |
|---|---|
| human serum (heat inactivated) | Fold decrease in MIC |
| HuLYZ, 10 μg/mL | 2 |
| HSA, 40 mg/mL | 4 |
| HuLYZ, 10 μg/mL + HSA 40 mg/mL | 16 |
| RabSA, 40 mg/mL | 4 |
| RatSA, 40 mg/mL | 2 |
| MouseSA, 40 mg/mL | 1 |

Interaction of PlySs2 (CF-301) with HSA in Human Serum

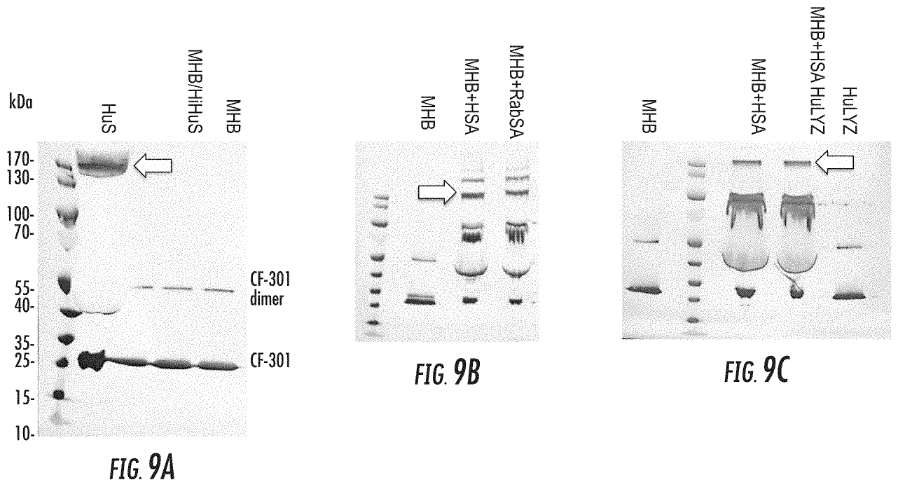

A western blot analysis was performed with anti-PlySs2 (CF-301) antibodies to detect PlySs2 (CF-301) in the MIC wells of conditions with a blood effect (i.e., human serum) and without a blood effect (i.e., MHB and MHB supplemented with 50% heat-inactivated human serum [MHB/HiHuS]). In the presence of bacterial cells, PlySs2 (CF-301) forms a band at high molecule weight (˜150 kDA) in human serum, but not in either MHB or MHB/HiHuS (FIG. 9A). The ˜150 kDA band is distinct from the PlySs2 (CF-301) monomers and dimers (and trimers) that are normally detected. Interestingly, the ˜150 kDA band is not observed when PlySs2 (CF-301) is incubated in human serum without bacterial cells (data not shown).

The effect of adding either HSA or RabSA (either at 40 mg/mL) to MHB was examined. The addition of either HSA or RabSA partially establishes blood effect in MHB (see above), and does result in the appearance of the ˜150 kDa band in the presence of bacterial cells (FIG. 9B). The addition of HuLYZ (at 10 μg/mL) is not associated with the formation of the ˜150 kDA band (FIG. 9C).

The nature of the ˜150 kDa band formed in human serum was examined by mass spectrometry. In addition to other proteins normally found at 150 kDa, the most abundant fragment signal detected was from human serum albumin (data not shown).

Serum Lipids are Required for the Potentiation Effect of PlySs2 (CF-301)

Our observation that delipidated human serum does not synergize with PlySs2 (CF-301) (TABLE 3) suggests that fatty acids (FAs) are required as part of the mechanism by which PlySs2 (CF-301) synergizes with HSA in serum. Most of the free FA in circulation is bound to HSA (24) and the process of delipidation, while not altering HSA levels, does reduce free FA levels by approximately one-third (Sacks F M et al (2009) J Lipid Res 50(5):894-907). To confirm that low FA levels are responsible for the inability of PlySs2 (CF-301) to synergize in delipidated serum, we determined the effect of introducing two of the more common FAs in circulation, oleate and palmitate t(Richieri G V & Kleinfeld A M (1995) J Lipid Res 36(2):229-240), on the performance of delipidated serum in the MIC assay format. The addition of either oleate or palmitate, at a physiological concentration of 0.625 mg/mL resulted in 8- and 16-fold decreases in the PlySs2 (CF-301) MIC, respectively, with no further decreases associated with the concomitant addition of both lipids (Table 12). Considering that the FAs alone (outside of the serum context) have no impact on PlySs2 (CF-301) activity (data not shown), it is likely that the effect is mediated through HSA. The effect is also likely to also reflect a direct interaction between HSA and PlySs2 (CF-301), based on the understanding that FAs bound to high-affinity sites on HSA act to promote additional binding activities to drugs and other compound (Yang F et al (2014) Int J Mol Sci 15(3):3580-3595).

TABLE 12
Enhancement of PlySs2 (CF-301) MIC values in delipidated
human serum supplemented with fatty acids

| | Fold decrease in CF-301 |
|---|---|
| Supplementation | MIC compared to media alone |
| Oleate (0.625 mg/mL) | 8 |
| Palmitate (0.625 mg/mL) | 16 |
| Palmitate + Oleate | 16 |

Cell Surface-Binding Studies Using HuLYZ and PlySs2 (CF-301)

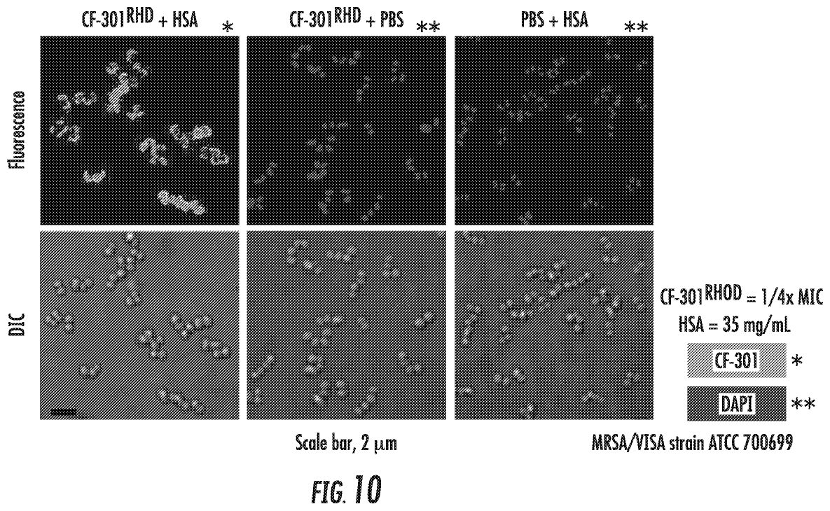

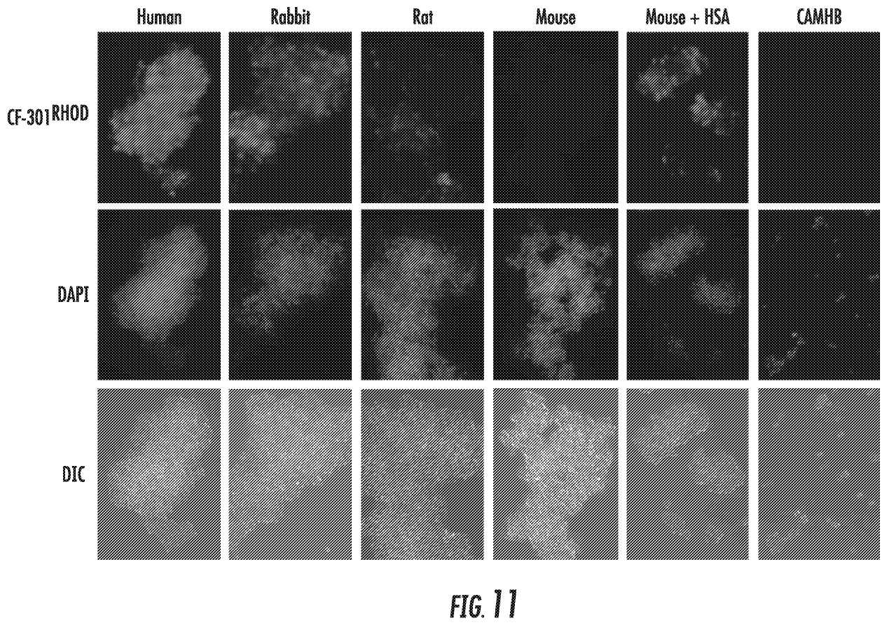

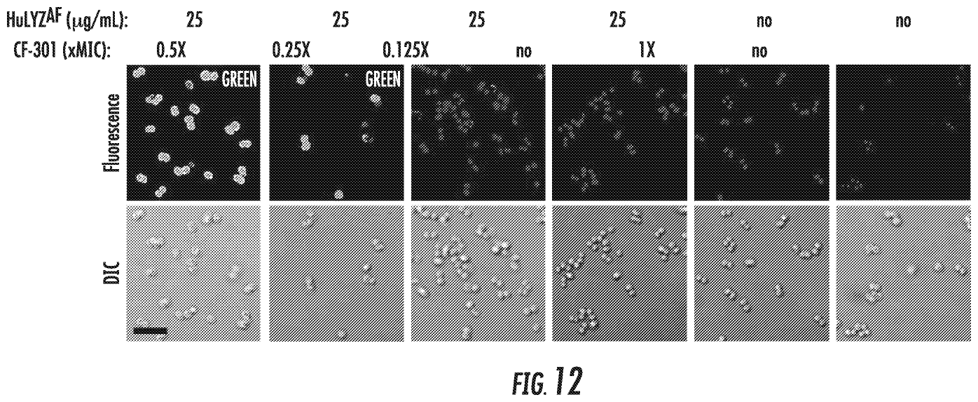

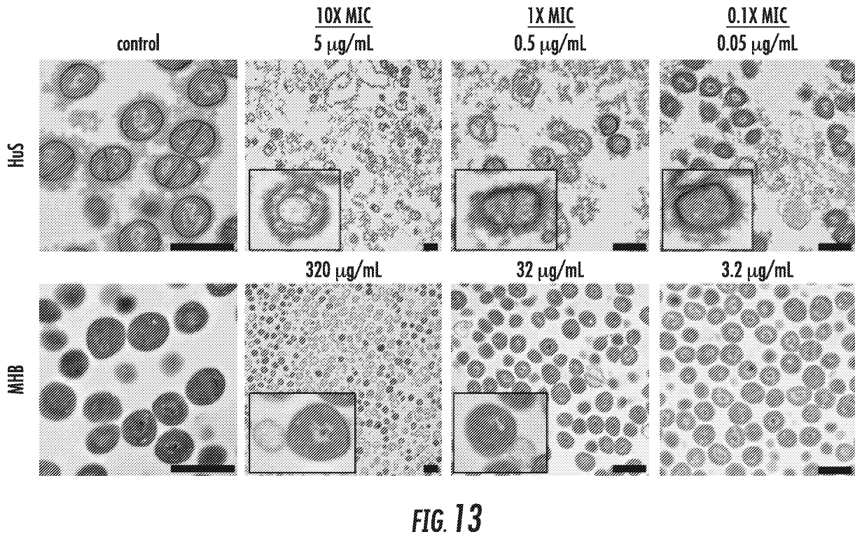

We used confocal microscopy to test the binding of rhodomine-labeled PlySs2 (CF-301) (CF-301^{RHD}) to the surface of S. aureus strain ATCC 700699 that has been pretreated with HSA at 40 mg/mL (FIG. 10). At an amount corresponding to 0.25×MIC, the CF-301^{RHD} construct showed extensive labeling of the staphylococcal cell wall in cells pretreated with HSA. In the absence of the HSA pretreatment, no labeling was observed. The HSA pretreatment did not enable binding of the fluorphore-tagged lysins PlyG and PlyC (specific for the surface of B. anthracis and S. pyogenes, respectively) to the staphylococci, confirming the specificity of the HSA activity (data not shown).

The effect of preincubation of staphylococci with multiple different serum types and MHB on subsequent labeling with a 0.25×MIC amount of CF-301^{RHD} was also examined by fluorescence microscopy (FIG. 11). Only the preincubations with either human serum or rabbit serum resulted in extensive labeling of S. aureus strain MW2. Preincubation with rat serum, mouse serum or MHB alone resulted in poorly labeled cells observed only with longer exposure times. The supplementation of mouse serum with HSA did restore high-level binding and fluorescence.

Based on the ability of HuLYZ to synergize with PlySs2 (CF-301), we next used confocal microscopy to test the binding of Alexa Fluor-labeled HuLYZ (HuLYZ^{AF}) to staphylococci pretreated with a sub-MIC range of CF-301 (FIG. 12). The HuLYZ^{AF} extensively labeled the cell wall of bacteria pretreated with either a 0.5× or 0.25×MIC amount of PlySs2 (CF-301). In the absence of pretreatment with PlySs2 (CF-301), no labeling was observed. Furthermore, the PlySs2 (CF-301) pretreatments did not facilitate the binding of either the PlyG or PlyC lysins (data not shown).

Visualization of PlySs2 (CF-301) Lytic Activity in Human Serum

Staphylococci were treated with a range of PlySs2 (CF-301) concentrations for 10 minutes in either human serum or MHB before analysis by transmission electron microscopy (TEM). While bacteriolytic activity was observed only at the highest concentration of PlySs2 (CF-301) in MHB (i.e., 5 μg/mL), widespread evidence of lysis was observed in human serum at all concentrations over a 100-fold range (FIG. 13). In addition to the more rapid bacteriolysis in serum, the lytic event was visually different in comparison to the event in MHB. In human serum, all bacteria are encased in a proteinaceous sheath presumably consisting of host proteins including HSA. Within the sheath, the lysing bacteria are distinguished by a circumferential dissolution of the electron dense cell wall material and, interestingly, the bacterial debris appears to remain ensheathed. In contrast, the lytic event in MHB appears as the classic cytoplasmic membrane bubbling and extrusion just prior to lysis.

In Vivo Evidence of a Strong Blood Effect on PlySs2 (CF-301) Activity

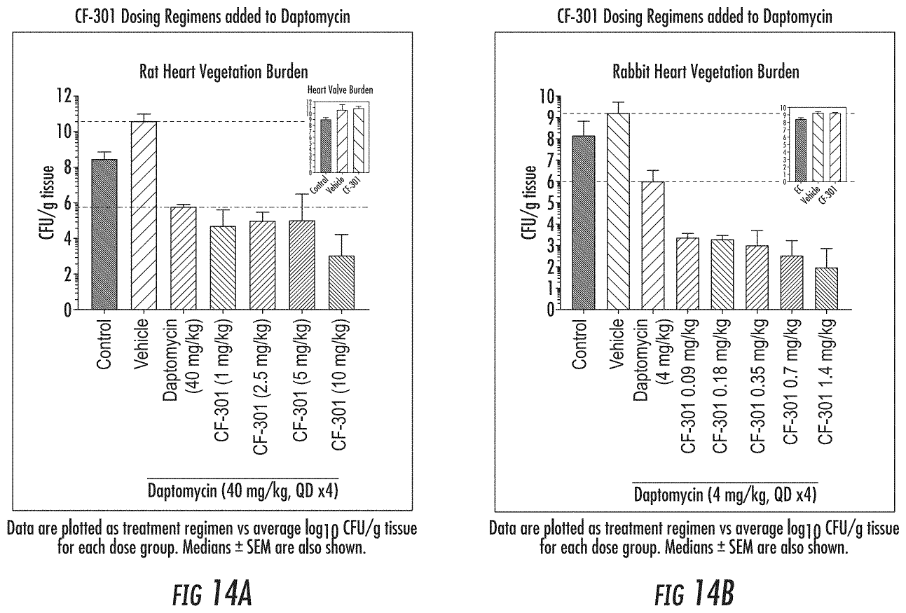

The efficacy of PlySs2 (CF-301) in addition to DAP was investigated using the rat and the rabbit models of infective endocarditis due to MRSA strain MW2. The in vivo studies indicated that PlySs2 (CF-301) in addition to DAP was more effective in the treatment of IE in the rabbit model compared to the rat model (FIG. 14). In the rat model, a total dose of 10 mg/kg of PlySs2 (CF-301) administered in addition to the human therapeutic dose (HTD) equivalent of DAP results in ˜3 log 10 drop in CFU/g in heart valve vegetation. The same 3 log 10 decrease in the bacterial densities as compared to DAP treatment alone is achieved in the rabbit model after administration of a total dose of ≥0.09 mg/kg of PlySs2 (CF-301) in addition to DAP below the HTD equivalent. The difference of PlySs2 (CF-301) efficacy in the two models is even more significant considering that that the rat model used a human equivalent dose of DAP whereas in the rabbit model PlySs2 (CF-301) was combined with a dose lower than the HTD equivalent of DAP.

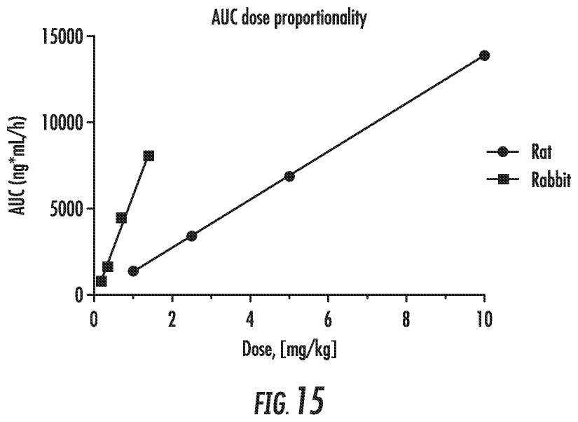

In the experimental rat IE model, an estimate AUC/MIC ratio of ≥0.87, attained at the 10 mg/kg PlySs2 (CF-301) dosing level in addition to DAP, was required to achieve maximal efficacy (3 log 10 drop in CFU/g in heart valve vegetation relative to DAP alone) (TABLE 13 and FIG. 15). On the contrary, similar AUC/MIC value was obtained in the rabbit model after PlySs2 (CF-301) at the dose of 0.18 mg/kg. Overall the in vivo studies demonstrated a ˜50 fold higher dose required for a similar bactericidal effect in rats (10 mg/kg) compare to rabbits (0.18 mg/kg).

TABLE 13
Simulated PlySs2 (CF-301) AUC and AUC/MIC Values for
Various PlySs2 (CF-301) Doses in Rats and Rabbits

| | Rat PK/PD | | | | Rabbit PK/PD | | | |
|---|---|---|---|---|---|---|---|---|
| Dose (mg/kg) | 1.0 | 2.5 | 5 | 10 | 0.18 | 0.35 | 0.7 | 1.4 |
| AUC (ng*mL/h) | 1352 | 3379 | 6855 | 13883 | 778 | 1631 | 4434 | 8031 |
| AUC/MIC^{$} | 0.08 | 0.21 | 0.43 | 0.87 | 0.78 | 1.6 | 4.4 | 8.0 |
| AUC/MIC^{$$} | 2.6 | 6.8 | 13.6 | 27.6 | 1.42 | 3.2 | 8.8 | 16.0 |

^{$}MIC in Rat Serum: 16 μg/mL; Rabbit Serum: 1 μg/mL
^{$$}MIC AST medium: 0.5 μg/mL

A listing of bacterial strains used in the studies herein is provided below in TABLE 14.

TABLE 14

| Organism, strain (resistance phenotype) | Source |
|---|---|
| Staphylococcus aureus, MW2 (MRSA) | NARSA |
| Staphylococcus aureus, NRS 23 (VISA) | NARSA |
| Staphylococcus aureus, NRS 77 (MSSA) | NARSA |
| Staphylococcus aureus, NRS 100 (MRSA) | NARSA |
| Staphylococcus aureus, NRS 153 (MSSA) | NARSA |
| Staphylococcus aureus, NRS 162 (MSSA) | NARSA |
| Staphylococcus aureus, NRS 271 (MRSA, LRSA) | NARSA |
| Staphylococcus aureus, ATCC BAA-42 (MRSA) | ATCC |
| Streptococcus pyogenes, ATCC BAA-946 | ATCC |
| Staphylococcus aureus, ATCC 43300 (MRSA) | ATCC |
| Staphylococcus aureus, ATCC 700699 (VISA) | ATCC |
| Staphylococcus aureus, ATCC 700698 (VISA) | ATCC |
| Staphylococcus aureus, HPV 107 (MRSA) | Vincent A. Fischetti |
| Staphylococcus aureus, CAIRD 456 (MRSA) | David P. Nicolau |
| Staphylococcus aureus, JMI 227 (MRSA) | JMI Laboratories |
| Staphylococcus aureus, JMI 1280 (MRSA) | JMI Laboratories |
| Staphylococcus aureus, JMI 4789 (MRSA) | JMI Laboratories |
| Staphylococcus aureus, JMI 5675 (MRSA) | JMI Laboratories |

Abbreviations: ATCC, American Type Culture Collection; CAIRD, Center for Anti-Infective Research and Development; LRSA, linezolid-resistant S. aureus; MSSA, methicillin-sensitive S. aureus; MRSA, methicillin-resistant S. aureus; NARSA, Network on Antimicrobial Resistance in S. aureus (now BEI Resources); VISA, vancomycin-intermediate S. aureus.

Discussion

In this study, the activity of PlySs2 (CF-301) and various other antibacterial lysins in Mueller Hinton Broth (a standard testing medium) was compared with that determined in biologically relevant media types including human whole blood, plasma, and serum.

The substantial ability of PlySs2 (CF-301) to activate and synergize with elements of human blood to potentiate an enhanced level of antistaphylococcal activity over that predicted from AST following standardized procedures (e.g., CLSI) using CAMHB reference broth is described and demonstrated herein. Our findings were initially based on in vitro time-kill assays demonstrating reductions of ≥100-fold in the minimum sterilizing concentration of PlySs2 (CF-301) against 11 staphylococcal strains in blood matrices from up to 34 different sources compared to CAMHB. We further demonstrated a ≥32-fold reduction in the MIC₉₀ against 171 S. aureus isolates tested in human serum and a consistently low level of MIC variability in the blood, serum and plasma from 61 different human sources. In combinations with either daptomycin or vancomycin in time-kill and/or checkerboard formats, we observed synergistic activities in human serum at PlySs2 (CF-301) concentrations up to 160 times lower than in CAMHB. Overall, these results support the potential effectiveness of PlySs2 (CF-301) as an intravenously administered antimicrobial agent for the treatment of S. aureus bacteremia and endocarditis.

Furthermore, our findings implicate synergy between PlySs2 (CF-301) and two specific components of human blood (i.e., lysozyme and albumin), with no apparent (single agent) intrinsic antistaphylococcal activity, as a key factors associated with enhanced activity and efficacy. The unique ability of PlySs2 (CF-301) to activate and synergize with otherwise dormant bystanders (with respect to killing staphylococci) has important implications for the medicinal use of PlySs2 (CF-301) and the measurement of its antimicrobial activity. Furthermore, our findings distinctly contrast with the general understanding that, for many systemically-delivered conventional, small molecule antibiotics, protein binding in circulation (primarily to albumin) serves to reduce drug activity (Zeitlinger M A, et al. (2011) Antimicrob Agents Chemother 55(7):3067-3074; Schmidt S, et al. (2008) Antimicrob Agents Chemother 52(11):3994-4000; Burian A, et al. (2011) J Antimicrob Chemother 66(1):134-137; Stratton C W & Weeks L S (1990) Diagn Microbiol Infect Dis 13(3):245-252; Beer J, Wagner C C, & Zeitlinger M (2009) AAPS J 11(1):1-12; Hegde S S, et al. (2004) Antimicrob Agents Chemother 48(8):3043-3050; Garonzik S M, et al. (2016) PLoS One 11(6):e0156131).

In accordance with the studies and results herein, MIC data collected in serum may be more appropriate for predicting in vivo activity and for the use in in vivo studies preceding clinical trials for lysin polypeptides, particularly lysin polypeptides such as PlySs2 (CF-301) lysin. In particular, based on the present studies, data collected in serum or with added serum components, may be more appropriate for PlySs2 (CF-301), Sal lysin, lysostaphin, etc as provided herein, which lysins have an SH3-type binding domain, or other lysins, chimerics, constructs having an SH3-binding domain. In fact, concentrations of certain peptides and peptide antibiotics (abx) required for in vivo treatments may be lower than traditionally deduced from MICs determined in lab media. One improved approach to testing antibacterial activity is the use of biologically relevant concentrations of blood matrices.

There has been no previous description of a role for HSA in promoting antimicrobial activity in the synergistic manner described for PlySs2 (CF-301). Our assumptions regarding HSA are strongly based on the following observations: (i) synergy with PlySs2 (CF-301) in multiple assay formats including checkerboards, time-kills, and the lytic assay; (ii) the ability to largely reconstitute the serum effect with PlySs2 (CF-301) in a media like CAMHB/HiHuS and CAMHB; (iii) a putative interaction with PlySs2 (CF-301) detected by western blot analysis; and (iv) the promotion of PlySs2 (CF-301) binding to the staphylococcal cell surface detected by microscopy. Additional support comes from the use of albumins from different species to replace HSA in reconstitution experiments. In particular, when rabbit SA is used at a physiological concentration of 40 mg/mL, it can mimic the activity of HSA. Both rat SA and mouse SA can only reconstitute the HSA effect, at the supraphysiological concentration of 40 mg/mL. The relatively low physiologic SA concentrations in rodents of 20 mg/mL, which is half the normal physiologic concentration of 40 mg/mL in rabbits and humans, may at least partially explain the inability of mouse and rat serum to serve as substrates for high-level PlySs2 (CF-301) activity.

Our experiments with delipated serum, in particular with the addition of oleate or palmitate to reconstitute the synergistic effect, also suggest a direct interaction between PlySs2 (CF-301) and HSA as part of the mechanism for enhanced activity. The circulating HSA monomer is commonly complexed with lipids and there are at least 7 high- and intermediate-affinity FA binding sites that can, when bound to FAs, modify and alter interactions with antibiotics and other molecules (Yang F, Zhang Y, & Liang H (2014) Int J Mol Sci 15(3):3580-3595). That such an interaction occurs with PlySs2 (CF-301) is also supported by our western blot data showing the formation of SDS-resistant high-molecular weight aggregates (i.e., ˜95 kDA and ˜150 kDA) only in the presence of HSA in conditions associated with a PlySs2 (CF-301) MIC of 1 μg/mL. The PlySs2 (CF-301) aggregates do not form in the absence of HSA, in conditions associated a PlySs2 (CF-301) MIC of 32 μg/mL. The potential relationship between aggregate formation, HSA binding, and high-level PlySs2 (CF-301) activity again stands in contrast to antibiotics, for which binding to serum proteins results in diminished activity.

Staphylococcus aureus expresses a large albumin-binding protein, Ebh, on its surface which contributes to survival in blood and the overall pathogenesis of staphylococcal infections (Cheng A G, Missiakas D, & Schneewind O (2014) J Bacteriol 196(5):971-981). Albumin-binding proteins are, in fact, found on a range of pathogenic microorganisms that are theorized to adsorb HSA as part of a survival strategy in host tissues (Egesten A et al (2011) J Biol Chem 286(4):2469-2476). These findings are in agreement with our observation, based on electron microscopy, showing the rapid accumulation of a dense proteinaceous surface layer on S. aureus in human serum, possibly consisting of albumin and/or other blood components. The layer forms a visible sheath around the staphylococci that modifies the visual manifestation of PlySs2 (CF-301) mediated bacteriolysis (compared to the event in CAMHB) and ultimately results in bacterial ghost-like structures (Wu X, et al. (2017) Foodborne Pathog Dis 14(1):1-7) with often intact cell envelopes encased in a matrix of possibly host-derived material. The encasement of bacterial debris and fragments (formed post-lysis) may also play an important role in mitigating the potential risk for pro-inflammatory responses associated the free release of these fragments into the bloodstream of the host. Furthermore, encasement of bacteria and PlySs2 (CF-301) in human HSA matrices may also reduce the risk of potentially deleterious immunologic reactions. Overall, our findings support the hypothesis that the natural ability of staphylococci to coat themselves with HSA in the bloodstream, and thus evade human immune surveillance, may be their Achilles' heel with respect to the binding capacity of HSA for PlySs2 (CF-301), which leads to enhanced bacteriolysis. In other words, the mechanism of synergy between PlySs2 (CF-301) and HSA is based on improved accumulation kinetics for PlySs2 (CF-301) at the bacterial cell surface mediated by HSA, and resulting in more rapid and efficient bacterial killing by PlySs2 (CF-301).

The results presented here also allow for conclusions to be drawn regarding the ability of PlySs2 (CF-301) to activate lysozyme against S. aureus in human serum. First, there is a general understanding that mature S. aureus peptidoglycan is resistant to HuLYZ activity by virtue of O-acetylation at the C-6 position of cell wall N-acetylmuramic acid (Bera A et al (2005) Mol Microbiol 55(3):778-787; Bera A et al (2006) Infect Immun 74(8):4598-4604). Accordingly, we observed no antistaphylococcal activity whatsoever for HuLYZ tested alone over a wide range of concentrations in multiple different AST formats. We did, however, observe distinct, synergistic antimicrobial activity of HuLYZ when tested at physiological concentrations in combinations with PlySs2 (CF-301) in time-kills, checkerboard assays, and the lytic assay. While the contribution of HSA to the synergistic effect is more substantial based on the in vitro reconstitution experiments, HuLYZ was consistently required for the full reconstitution effect and was observed to dramatically increase the extent of PlySs2 (CF-301)-mediate surface labeling of staphylococci by deconvolution microscopy. Taken with the proposed activity of HSA, our model holds that PlySs2 (CF-301) accumulates at the cell surface in a preferential manner by virtue of interactions with HSA and, independent of this, via the activation of HuLYZ. While the exact nature of this HuLYZ activity is unknown, one possible explanation holds that PlySs2 (CF-301)-mediated cleavage of peptidoglycan initiates access of HuLYZ to nascent peptidoglycan formed prior to 0-acetylation and that the subsequent hydrolytic activity of HuLYZ promotes more PlySs2 (CF-301) binding as bacteriolysis proceeds.

To understand the in vivo efficacy profile of PlySs2 (CF-301) in the intended clinical indication of staphylococcal bacteremia and endocarditis, we chose to conduct experiments in two species (rabbits and rats) with observed differences in the capacity of PlySs2 (CF-301) to synergize with respective serum types in the ex vivo formats reported here. The IE model is well-established in both rabbits and rats (Abdelhady W et al (2017) Antimicrob Agents Chemother 61(2); Hady W A, Bayer A S, & Xiong Y Q (2012) J Vis Exp (64):e3863) and has a significant biofilm component that is highly relevant with respect to the intended clinical indication for PlySs2 (CF-301). In accordance with our ex vivo observations of potent PlySs2 (CF-301) synergy in rabbit serum but not in rat serum, we observed that a >50 fold higher dose of PlySs2 (CF-301) and a >20 fold higher AUC exposure was required to obtain a similar bactericidal effect in rats (10 mg/kg) compared to rabbits (0.18 mg/kg). These models provide further evidence as to the potential therapeutic implications of the ability of PlySs2 (CF-301) to activate and synergize with HSA and HuLYZ and support the anticipated efficacy of PlySs2 (CF-301) at the doses selected for therapeutic use in clinical trials evaluating PlySs2 (CF-301) for the treatment of S. aureus bacteremia including endocarditis.

Some prior studies have evaluated antibacterial effects in serum and plasma, however, the results and conclusions have been varied. The presence of human blood plasma was reported to increase the activity of antibacterial peptidomimetics (AMPs) such as alpha-peptide/beta-peptoid peptidomimetics vs E. coli (Hein Kristiansen et al 2013, Citterio et al 2016), leading to the hypothesis that synergy with blood components might be involved, however, this was not fully evaluated. The finding that peptidomimetics and PMB have lowered MICs by 2-16 fold in plasma led to the suggestion that potentiation by plasma could be caused by endogenous blood components such as complement, as heat inactivation did abolish the synergism. Heat inactivation caused a dramatic increase in the MIC. Other components of the plasma, it was suggested, could act in potentiation, including proteins of the complement cascade, which could explain why plasma gave rise to more potentiation with some AMPs than does serum. Unlike serum, plasma contains active clotting factors which may respond to the presence of bacteria.

Plasma enhanced the activity of PMB but not gentamycin and ampicillin. Other studies have reported that complement proteins can act in synergy with antimicrobial compounds such as PMB and AMPs. The potentiation effects are dependent on the mode of action of the agent since only compounds active on the cell membrane or envelope appear to be potentiated. Conclusion was made that coagulation proteins act with complement to potentiate activity.

Vaara et al 1984 evaluated an outer membrane-disorganizing peptide PBMN and found that the small cationic outer membrane disorganizing peptide PMBN sensitizes E. coli to serum bactericidal action, facilitates the insertion or binding of antibodies or other factors present in normal serum with the resultant activation of complement cascade. The PMBN mediated bactericidal activity of serum was abolished by heating. The effect was noted in humans, guinea pigs, and rabbits, with an intermediary effect in rats, and no effect in mice serum. Mice have been cited as being unique among mammals because their normal or hyperimmune serum or peritoneal fluid also lacks bactericidal action that are readily killed in the sera of other animals.

Pruul and McDonald 1992 assessed potentiation of antibacterial activity of azithromycin by normal human serum. In their studies, 40% serum in MIC assays causes a 15-fold decrease in MIC versus the bacteria S. aureus. The enhancement, however, was not inhibited by heat inactivation, showing that it was not heat sensitive. Also they found no difference for complement inactivated serum or antibody-depleted serum. Also, the additional of albumin (Cohn Fraction V) (to 70 mg/ml) to TSB testing broth did not restore activity. The effect was limited to gram negative species and was not observed with staphylococci.

Serum enhancement factors of human serum may include serum lipoprotein, changes in pH, antibacterial peptides. Changes in the bacterial growth rate and the composition of bacterial surfaces brought about by the serum factors may modify bacterial membrane permeability, alter antibiotic accumulation kinetics, or enhance antibiotic binding.

Given the substantial diversity of innate immune molecules in an animal host, it is possible that PlySs2 (CF-301) activity could synergize with and improve the overall effectiveness of many other peptides.

Based on the present studies, we suggest the following model to explain biologically the activity enhancement. In the blood, S. aureus bacteria is coated with serum proteins including HSA in order to evade the immune system (the HSA binding proteins on S. aureus are known and have been previously described). The HSA binding comes at a price, including decreased cross-linking. By virtue of PlySs2 (CF-301) (and other SH3 binding type lysins) interaction with HSA in the presence of bacteria, the lysin concentrates at the bacterial surface. This concentration, combined with the decreased cross-linking imposed by HSA binding, enhances the antimicrobial activity of PlySs2 (CF-301). In the case of human lysozyme (HuLYZ), which is ordinarily ineffective against S. aureus bacteria, the enhanced activity of PlySs2 (CF-301), combined with the decreased cross-linking, facilitates HuLYZ binding and activity against nascent peptidoglycan that remains sensitive to HuLYZ. The combined activity of HSA, HuLYZ and PlySs2 (CF-301) enables the blood effect.

Sensitization of S. aureus to HuLYZ is newly recognized and not previously observed. A hallmark of S. aureus is resistance to lysozyme which is central to its strategy for immune evasion. PlySs2 (CF-301) treatment circumvents this and renders lysozyme active against S. aureus.

Thus, the present experiments demonstrate that PlySs2 (CF-301) is a very efficient anti-staphylococcal agent based on its innate hydrolytic activity and its ability to potentiate the antibacterial activity of lysozyme. In addition, PlySs2 (CF-301) synergizes and potentiates with HSA and/or utilizes HSA binding to bacterial cells to bring PlySs2 (CF-301) to bacteria and/or effectively concentrate the PlySs2 (CF-301) at bacterially infected sites.

Our data demonstrate that the human blood environment can synergize with and greatly enhance or potentiate the antimicrobial activity of the anti staphylococcal lysin PlySs2 (CF-301), as well as other exemplary SH3-type binding domain containing lysins, as a result of synergistic interactions with both an innate immune effector, lysozyme, and the most abundant serum protein, albumin. This study highlights the remarkable adaptability of this enzyme class to accommodate widely different peptidoglycan substrates. This observation underscores the adaptability of microorganisms in response to antibiotic challenge and demonstrates the susceptibility of established antibiotics long thought to be exempt from resistance.

Materials and Methods

Bacteria, Media and Growth Conditions. A subset of the bacterial strains used in this study is described in Table S8. The 171 S. aureus strains used in Table 1 were previously described (4), including 74 MSSA and 75 MRSA clinical isolates from 2011 that were obtained from JMI Laboratories (North Libery, Iowa). Bacteria were cultivated on either BBL™ Trypticase™ soy agar with 5% sheep blood (TSAB; Becton, Dickinson & Company [BD]), BBL™ Mueller Hinton II Broth, Cation Adjusted (CAMHB; BD), or Brain Heart Infusion Broth (BHI; BD) unless otherwise indicated. With the exception of HyClone™ Fetal Bovine Serum (0.1 micron filtered; GE Healthcare Lifesciences), the source, description and lot number of all human and animal blood matrices tested are described in Table S1 and Table S2. Staphylococci were grown at 37° C. with aeration unless otherwise indicated.

Reagents. Lysin PlySs2 (CF-301) was expressed, purified and stored as previously described (4). Human anti-PlySs2 (CF-301) IgG3 monoclonal antibody (expressed and purified at ContraFect Corporation) and mouse anti-human IgG3 heavy chain antibody, AP conjugate (ThermoFisher 05-3622) were used as primary and secondary antibodies at 1:1,000 dilution for Western blots. All agents (and vendor sources) tested in combination with PlySs2 (CF-301) were as follows: D-Defensin-3, human (Anaspec); Leap-1, human (Anaspec); Leap-2, human (GenScript); LL-37, human (Anaspec); LL-18-37 (Anaspec); histatin-5, human (Anaspec); HNP-1, human (Anaspec); HNP-2, human (Anaspec); Human Platelet Factor IV 18 (Anaspec); lactoferrin, human milk (Sigma-Aldrich); lactoferrin, bovine colostrum (Sigma-Aldrich); Lactoferricin H, human (Anaspec); human lysozyme, recombinant expressed in rice (Sigma-Aldrich); lysozyme, hen egg (Sigma-Aldrich); lysozyme, human neutrophil-derived (AVIVA Biosciences); lysozyme, human neutrophil-derived (RayBiotech); human serum albumin, recombinant expressed in rice (Sigma-Aldrich); human serum albumin, fraction V (Sigma-Aldrich); human serum albumin, fraction V, fatty acid-free, globulin-free (Sigma-Aldrich); human serum albumin, recombinant expressed in yeast (Albumin Bioscience); mouse serum albumin, recombinant expressed in yeast (Albumin Biosciences); rabbit serum albumin (Sigma-Aldrich); and rat serum albumin (Sigma-Aldrich). Sodium oleate, sodium palmitate, vancomycin hydrochloride, daptomycin, proteinase K-agarose from Tritirachium album were obtained from Sigma-Aldrich. For microscopy, DAPI, Alexa Fluor® 488, and NHS-Rhodamine were obtained from Thermo Fisher Scientific. The labeling and purification of PlySs2 (CF-301) conjugated to NHS-Rhodamine and HuLYZ conjugated to Alexa Fluor 488 were performed as described by the manufacturer's protocol. Non-reacted fluorophores were removed using PD-10 desalting columns (GE Healthcare) and labeling efficiencies were determined to be >80% in each case. The activity of PlySs2 (CF-301)-rhodamine (CF-301^{RHOD}) was confirmed to be equivalent to PlySs2 (CF-301) using the standard MIC assay. The activity of HuLYZ-Alexa Fluor 488 (HuLYZ^{AF}) was confirmed to be equivalent to HuLYZ using a drop dilution assay on an 1% agarose surface impregnated with 1 mg/ml of peptidoglycan from Micrococcus luteus (Sigma-Aldrich). The production, purification and use of GFP-labeled PlyG (PlyG^{GFP}) and Alexa Fluor488-labeled PlyC (PlyC^{AF}) were previously described (38, 39).

Time-kill assays. Bactericidal activities were tested according to the CLSI method (52). Assays were performed in CAMHB or the indicated (undiluted) blood matrix using a bacterial inoculum of 5×10⁵ CFU/mL in 125-mL glass Erlenmeyer flasks with agitation. Indicated agents were tested across a 10-fold range of concentrations. For daptomycin, CAMHB cultures were supplemented with 50 μg/mL Ca²⁺. Growth controls with buffer-alone were always included. Immediately before treatment and at indicated time intervals thereafter up to 24 hours, culture aliquots were removed and diluted in activated charcoal (to impede or halt drug activity). A series of 10-fold dilutions of the inactivated cultures were then plated on TSAB and incubated at 37° C. for 24 hours prior to colony enumeration. Bactericidal activity was defined as a decrease of ≥3 log 10 CFU/mL relative to the initial inoculum.

MIC assays. MIC values were determined by broth microdilution using the CLSI reference method (62) in either CAMHB or the indicated blood matrices. The CAMHB/HiHuS was prepared by supplementing CAMHB with 50% human serum and then filtering through a Microcon Centrifugal Filter Unit (Amicon Ultra-15; Millipore) with 50 kDa cut off before incubation at 70° C. for 20 minutes to completely inactivate component/s responsible for synergy with PlySs2 (CF-301). For the supplementation of delipidated serum with fatty acids, 50 mg/mL stock solutions of either oleate in H₂O or palmitate in 100% ethanol were prepared and added to the serum to achieve a final concentration of 0.625 mg/mL; the supplemented serum was then incubated at 37° C. for 1 hour prior to use to promote fatty acid binding to HSA. Colorimetric determination of PlySs2 (CF-301) MIC values was performed using AlamarBlue® (Thermo Fisher Scientific) exactly according to the manufacturer's protocol. The analysis of PlySs2 (CF-301) activity in human serum pretreated for 3 hours with proteinase K-agarose beads was performed according to the manufacturer's protocol (Sigma-Aldrich). As a control for protease carry-over after the removal of proteinase K-agarose beads, the treated serum was diluted 3:4 into untreated serum prior to MIC determination; the addition of untreated serum was expected to restore the synergistic effect only if there was no carry-over of either unbound proteinase K or the proteinase K-agarose beads.

Lytic assays. Overnight cultures of MRSA strain MW2 were diluted 1:100 in BHI and grown for 2.5 hours at 37° C. with aeration. The exponential phase cells were washed, concentrated 10-fold in 20 mM phosphate buffer (pH 7.4), and split in equal aliquots to which either HSA (Albumin Biosciences) or human neutrophil-derived lysozyme (AVIVA Bioscience) over a range of concentrations was added. For each concentration of HSA or lysozyme, 0.1 mL of the mixture was aliquoted in duplicate to a 96-well, flat-bottom, non-tissue culture treated microtiter plates (BD). The lytic reaction was then started by adding to all wells 0.1 mL PlySs2 (CF-301) (in phosphate) to a final concentration of 4 μg/mL. Control wells were included with PlySs2 (CF-301), HSA, or HuLYZ alone at appropriate concentrations. Samples were mixed and optical density at 600 nm (OD₆₀₀) was followed for 15 minutes at room temperature in a SpectraMax M5 Microplate Reader (Molecular Devices).

A variation of the lytic assay was performed to determine PlySs2 (CF-301) specific activity. Exponential phase MW2 cells were prepared as above and divided in 4 mL aliquots containing either phosphate buffer (reactions without agents added, with cells alone) or HSA (Albumin Biosciences) and/or human lysozyme (AVIVA Bioscience). The PlySs2 (CF-301) (starting at 0.5 mg/mL) was 2-fold serially diluted across columns 1 through 11 of a 96 well plate with 20 mM Phosphate pH 7.4 at a volume of 0.1 mL. Column 12 contained no enzyme, only buffer, and was used as assay control well. Bacterial cells mixed with either buffer, HSA, lysozyme or HSA combined with HuLYZ were added as 0.1 mL aliquots to each well of three rows of serially diluted PlySs2 (CF-301). Each plate was read at 600 nm for 15 minutes (shaking for 3 seconds between reads) at room temperature using a microplate reader as above. The specific activity in Units/mg of PlySs2 (CF-301) was determined based on the PlySs2 (CF-301) dilutions displaying curves just above and below the optical density that is 50% of the buffer control at 15 minutes.

Checkerboard assays. The checkerboard assay was performed as described (66) and is adapted from the CLSI method for broth microdilution (62). Checkerboards were prepared by first aliquoting in each column of a 96-well polystyrene microtiter plate the same amount of PlySs2 (CF-301) diluted 2-fold along the x axis. In a separate plate, correspondent rows were prepared in which each well had the same amount of another agent diluted 2-fold along the y-axis. The dilutions were then combined, so that each column had a constant amount of PlySs2 (CF-301) and doubling dilutions of the second agent, while each row had a constant amount of the second agent and doubling dilutions of PlySs2 (CF-301). Each well thus had a unique combination of PlySs2 (CF-301) and the second agent. Bacteria were added to each well at concentrations of ˜5×10⁵ CFU/mL in CAMHB or human serum (pooled male, type AB, sterile-filtered, US origin; Sigma-Aldrich). The MIC of each drug, alone and in combination, was recorded after 18 hours at 37° C. in ambient air. Results are expressed in terms of a ΣFIC index equal to the sum of the FICs for each drug; the FIC for a drug is defined as the MIC of the drug in combination divided by the MIC of the drug used alone. Both the ΣFICₘᵢₙ (lowest ΣFIC value obtained among all combinations) and ΣFIC_{AVG} (the average ΣFIC value of three consecutive drug combinations) are reported for each paired agent. If the ΣFIC index is ≤0.5, the combination is interpreted as being synergistic; between >0.5 and ≤2 as additive; and >2 as antagonistic (16). Colorimetric determinations of PlySs2 (CF-301) MIC values were also performed using AlamarBlue® (Thermo Fisher Scientific), according to the manufacturer's protocol.

Synergy time-kill assays. Synergy time-kill curves were performed according to the method described by the CLSI (52). Strain MW2 was suspended in CAMHB with 50% HiHuS at a concentration of 5×10⁵ colony forming units (CFU)/mL and exposed to PlySs2 (CF-301) and/or daptomycin, HSA (Albumin Biosciences), and HuLYZ (AVIVA Bioscience) for 24 hours at 35° C. in ambient air with agitation. At timed intervals, culture samples were removed, serially diluted, and plated to determine CFU/mL. The resulting kill kinetic determinations are shown graphically by plotting log₁₀ CFU/mL versus time. Synergy is defined as a ≥2-log₁₀ decrease in CFU/mL between the combination and its most active constituent with the least active constituent tested at an ineffective concentration.

Fluorescence microscopy: Binding of PlySs2 (CF-301) to the Bacterial Cell Surface in Different Serum Environments. Mid-log phase MRSA strain MW2 was suspended at 1×10⁷ CFU/mL in either CAMHB or 100% serum from either human (Sigma-Aldrich), rabbit (Gibco), rat (BioreclamationIVT), or mouse (BioreclamationIVT) sources and incubated for 30 minutes at 37° C. An additional mouse serum sample was also tested, containing 40 mg/mL HSA (Albumin Biosciences). After the preincubation, bacteria were washed with 1× phosphate-buffered saline (PBS) and resuspended in 50 μl of PBS and attached to the surface of a poly-L-lysine-coated cover glass. The cells were washed and treated with CF-301^{RHOD} (2 μg/mL in PBS) for 10 minutes before washing and counterstaining with DAPI. Slides were mounted in 50% glycerol and 0.1% p-phenylenediamine in PBS, pH 8. Fluorescence microscopy was performed using a Nikon Eclipse E400 microscope, equipped with a Nikon 100×/1.25 oil immersion lens, and a Retiga EXi fast 1394 camera (QImaging). QCapture Pro version 5.1.1.14 software (QImaging) was used for image capture and processing.

Fluorescence microscopy: Enhancement of PlySs2 (CF-301) Binding in the Presence of HSA. An overnight culture of VISA strain ATCC 700699 was diluted 1:100 in CAMHB, grown to OD₆₀₀ of 0.6, and attached to the surface of poly-L-lysine-coated cover glass. The cells were washed with PBS and treated for 30 min at room temperature with 10 μl PBS containing HSA (Albumin Biosciences) or PBS alone. Duplicate samples were then supplemented with 1/10th of the original volume of PBS, or PBS containing NHS-rhodamine-labeled PlySs2 (CF-301), to a final concentration of 4 μg/mL (0.25×MIC). The cells were incubated for a further 30 min at room temperature, washed with PBS and fixed with 2.6% paraformaldehyde in PBS for 45 min at room temperature. The slides were then washed with PBS and mounted in PBS pH 8.0 containing 50% glycerol, and 0.1% p-phenylenediamine. DAPI was used as counter stain in all assay conditions. Deconvolution microscopy was performed using a DeltaVision image restoration microscope (Applied Precision/Olympus) equipped with CoolSnap QE cooled CCD camera (Photometrics). Imaging was done using an Olympus 100×/1.40 N.A., UPLS Apo oil immersion objective combined with a 1.5× optovar. Z-stacks were taken at 0.15-μm intervals. Images were deconvolved using the SoftWoRx software (Applied Precision/DeltaVision), corrected for chromatic aberrations, and presented as maximum intensity projections combining all relevant z-sections. In a complementary analysis, similar to that described above, the NHS-rhodamine-labeled PlySs2 (CF-301) was replaced with either 25 μg/mL PlyG^{GFP} or PlyC^{AF}. After treatment, the samples were visualized by fluorescence microscopy using a Nikon Eclipse E400 microscope, equipped with a Nikon 100×/1.25 oil immersion lens.

Fluorescence microscopy: Enhancement of HuLYZ Binding in the Presence of PlySs2 (CF-301). An overnight culture of VISA strain ATCC 700699 was diluted 1:100 in CAMHB, grown to OD₆₀₀ 0.6, and attached to the surface of poly-L-lysine-coated cover glass. The cells were washed with PBS and treated for 30 min at room temperature with 50 μl PBS containing PlySs2 (CF-301) at different concentrations or PBS alone. Duplicate samples were then supplemented with 1/10th of the original volume of PBS, or PBS containing Alexa Fluor 488-labeled HuLYZ to a final concentration of 10 μg/mL. The cells were incubated for a further 30 min at room temperature, washed with PBS and fixed with 2.6% paraformaldehyde in PBS for 45 min at room temperature. The slides were then washed with PBS and mounted in 20 mM Tris pH 8.0, 90% glycerol, 0.5% n-propyl gallate. DAPI was used as counterstain in all assay conditions. Deconvolution microscopy was performed using a DeltaVision image restoration microscope (Applied Precision/Olympus) equipped with CoolSnap QE cooled CCD camera (Photometrics) as described above. In a complementary analysis, Alexa Fluor488-labeled CF-301 was replaced with either 25 μg/mL PlyG^{GFP} or PlyC^{AF}. After treatment, the samples were visualized by fluorescence microscopy using a Nikon Eclipse E400 microscope, equipped with a Nikon 100×/1.25 oil immersion lens.

Electron Microscopy. Mid-log phase strain MW2 growing in either CAMHB or human serum (Sigma-Aldrich) was treated with the indicated concentrations of PlySs2 (CF-301) or buffer alone (control) at 37° C. for 15 minutes. The cells were then washed with 1× phosphate buffer (PB) and resuspended in a solution of 4% paraformaldehyde and 2% glutaraldehyde in 0.1 M cacodylate buffer (pH 7.4). The samples were post-fixed in 1% osmium tetroxide, block stained with uranyl acetate, and processed according to standard procedures by The Rockefeller University Electron Microscopy Service. Samples were visualized using a Tecnai™ Spirit BT Transmission Electron Microscope (FEI). The human serum was sterile-filtered and obtained from a pooled male population (˜70 subjects) of US origin with type AB blood.

Western Blot Analysis. The PlySs2 (CF-301) MIC well samples taken from the indicated media types were analyzed by Western Blot. Sample aliquots of 10 μl each were run on 4-12% Tris-Glycine Mini Gels (Novex™) and then transferred to a polyvinylidine fluoride (PVDF) membrane via electroblotting. The PVDF membranes were incubated with an anti-PlySs2 (CF-301)-specific Protein G affinity purified human IgG3 recombinant monoclonal antibody followed by a secondary monoclonal murine anti-IgG3-alkaline phosphatase conjugate detection antibody. Both primary and secondary antibodies were used at 1:1,000 dilutions. The membrane was stained with nitro-blue tetrazolium and 5 bromo 4 chloro 3′ indolyphosphate (NBT/BCIP) chromogenic substrate. The molecular weight of visible bands was determined by comparison to the bands of a molecular weight standard run in the same gel.

Rat infective endocarditis model. Sprague-Dawley rats (250-275 g), anesthetized with ketamine 87 mg/kg and xylazine 13 mg/kg cocktail via intraperitoneal injection (IP), underwent a standard indwelling transcarotid transaortic valve-to left ventricle catheterization. After 48 hours, animals were challenged with S. aureus strain MW2 (˜1×10⁵ colony forming units [CFU]/rat; IV) to induce endocarditis. At 24 hours post-infection, a cohort of rats was euthanized and heart valve vegetations were collected to determine initial tissue burdens (control group). The remaining rats were treated with either vehicle (saline; IV single dose) or DAP alone (40 mg/kg; subcutaneous injection (SQ); qd×4 days) or with DAP in addition to PlySs2 (CF-301). PlySs2 (CF-301) was administered IV as a single slow bolus (injection over 5-10 min) on the first day of treatment only, just after the initial DAP dose, at four dosing regimens (1, 2.5, 5, and 10 mg/kg). Treated animals were euthanized (sodium pentobarbital at 200 mg/kg by rapid IP push) 24 hours after the last DAP treatment (5 days post infection) and the cardiac valve vegetation was removed, weighed, homogenized and serially diluted in sterile PBS for quantitative culture onto TSAB. All culture plates were incubated at 37° C. for 24 hours, resulting colonies enumerated and expressed as log₁₀ CFU/g of tissue. Data for each organ for different treatment groups were calculated as median log₁₀ CFU/g of tissue ±95% CI.

Population PK modeling of PlySs2 (CF-301). Pharmacokinetic (PK) data was collected from previous non-clinical toxicology studies that included rats and dogs treated with PlySs2 (CF-301) at varying doses (41, 42). Post-dose plasma was collected over a timecourse and analyzed using a validated PlySs2 (CF-301) ELISA. The predicted AUC values for PlySs2 (CF-301) at doses of 1, 2.5, 5 and 10 mg/kg as a 10 minute intravenous infusion were derived from population PK modeling based on the nonclinical rat and dog PK experiments. For the current study, the previously reported AUC values were divided by the MIC value determined in rat serum (i.e., 16 μg/mL) for MRSA isolate MW2 to yield the calculated AUC/MIC ratio values reported in Table 13.

Rabbit infective endocarditis model. New Zealand White Rabbits (2.2-2.5 kg), anesthetized with a ketamine 35 mg/kg and xylazine 5 mg/kg cocktail via intramuscular injection (IM), underwent a standard indwelling transcarotid-transaortic valve-to-left ventricle catheterization (43). At 48 hours post catheter placement, animals were challenged IV with an inoculum of ˜2×10⁵ CFU of the S. aureus strain MW2 to induce infective endocarditis (IE). From previous studies, this inoculum has been shown to induce IE in >95% of catheterized animals. At 24 hours post-infection, a cohort of rabbits was euthanized and heart valve vegetations were collected to determine initial tissue burdens (control group). The remaining rabbits were treated with either vehicle (saline; IV single dose) or DAP alone (4 mg/kg IV; qd×4 days), PlySs2 (CF-301) alone or with DAP in addition to PlySs2 (CF-301). PlySs2 (CF-301) was administered IV as a single slow bolus (injection over 5-10 min) on the first day of treatment only, just after the initial DAP dose at four dosing regimens (0.09, 0.18, 0.35, 0.70 and 1.4 mg/kg). Treated animals were euthanized (sodium pentobarbital at 200 mg/kg by rapid IP push) 24 hours after the last DAP treatment (5 days post infection) and the cardiac valve vegetation was removed, weighed, homogenized and serially diluted in sterile PBS for quantitative culture onto TSAB. All culture plates were incubated at 37° C. for 24 hours, resulting colonies enumerated and expressed as log₁₀ CFU/g of tissue. Data for each organ for different treatment groups were calculated as median log₁₀ CFU/g of tissue ±95% CI.

Rabbit pharmacokinetics. New Zealand White Rabbits were dosed with PlySs2 (CF-301) (IV, slow bolus) at 0.18, 0.35, 0.07 or 1.4 mg/kg. After increasing amounts of time post dose, plasma was collected and analyzed using a validated PlySs2 (CF-301) ELISA in the manner described (63, 64). The predicted AUC values for PlySs2 (CF-301) at doses of 1, 2.5, 5 and 10 mg/kg as a 10 minute intravenous infusion were derived using WinNonLin (Data Not Shown). These values, divided by the MIC value (1 μg/mL in rabbit serum) of MRSA isolate MW2, resulted in calculated AUC/MIC ratio values reported in Table 8.

Daptomycin dose rationale in rabbits. Daptomycin dose response experiments were performed at doses ranging from 1 mg/kg to 10 mg/kg IV, once daily for 4 days in the rabbit IE model caused by S. aureus strain MW2. Daptomycin at 4 mg/kg, representing a dose below the human therapeutic dose equivalent, was chosen to explore the benefit of PlySs2 (CF-301) therapy in addition to daptomycin. In the rabbit IE model, a daptomycin dose of 4 mg/kg IV provided ˜2-3 log₁₀ reduction in bacterial burden compared to vehicle treated controls. Treated animals still had significant burdens of ˜5-7 log₁₀ providing significant dynamic range to observe the added effect of PlySs2 (CF-301) to this treatment regimen.

### REFERENCES

1. Wittekind M & Schuch R (2016) Cell wall hydrolases and antibiotics: exploiting synergy to create efficacious new antimicrobial treatments. Curr Opin Microbiol 33:18-24.
2. Nelson D C, et al. (2012) Endolysins as antimicrobials. Adv Virus Res 83:299-365.
3. ClinicalTrials.gov (2017) Safety, Efficacy and Pharacokinetics of CF-301 vs. Placebo in Addition to Antibacterial Therapy for Treatment of S. aureus Bacteremia. (clinicaltrials.gov/ct2/show/NCT03163446).
4. Schuch R, et al. (2014) Combination therapy with lysin CF-301 and antibiotic is superior to antibiotic alone for treating methicillin-resistant Staphylococcus aureus-induced murine bacteremia. J Infect Dis 209(9):1469-1478.
5. Schuch R, Khan B K, Raz A, Rotolo J A, & Wittekind M (2017) Bacteriophage Lysin CF-301, a Potent Antistaphylococcal Biofilm Agent. Antimicrob Agents Chemother 61(7).
6. Levison M E & Levison J H (2009) Pharmacokinetics and Pharmacodynamics of Antibacterial Agents. Infect Dis Clin North Amer 23(4):1-29.
7. Zeitlinger M A, et al. (2011) Protein binding: do we ever learn? Antimicrob Agents Chemother 55(7):3067-3074.
8. Schmidt S, Gonzalez D, & Derendorf H (2010) Significance of protein binding in pharmacokinetics and pharmacodynamics. J Pharm Sci 99(3):1107-1122.
9. Schmidt S, et al. (2008) Effect of protein binding on the pharmacological activity of highly bound antibiotics. Antimicrob Agents Chemother 52(11):3994-4000.
10. Zeitlinger M, et al. (2008) Plasma protein binding of fluoroquinolones affects antimicrobial activity. J Antimicrob Chemother 61(3):561-567.
11. Burian A, et al. (2011) Plasma protein binding may reduce antimicrobial activity by preventing intra-bacterial uptake of antibiotics, for example clindamycin. J Antimicrob Chemother 66(1):134-137.
12. Pruul H & McDonald P J (1992) Potentiation of antibacterial activity of azithromycin and other macrolides by normal human serum. Antimicrob Agents Chemother 36(1):10-16.
13. Citterio L, et al. (2016) Improved in vitro evaluation of novel antimicrobials: potential synergy between human plasma and antibacterial peptidomimetics, AMPs and antibiotics against human pathogenic bacteria. Res Microbiol 167(2):72-82.
14. Deslouches B, et al. (2005) Activity of the de novo engineered antimicrobial peptide WLBU2 against Pseudomonas aeruginosa in human serum and whole blood: implications for systemic applications. Antimicrob Agents Chemother 49(8):3208-3216.
15. Vaara M, Viljanen P, Vaara T, & Makela P H (1984) An outer membrane-disorganizing peptide PMBN sensitizes E. coli strains to serum bactericidal action. J Immunol 132(5):2582-2589.
16. Yan H & Hancock R E (2001) Synergistic interactions between mammalian antimicrobial defense peptides. Antimicrob Agents Chemother 45(5):1558-1560.
17. Yeaman M R & Yount N Y (2003) Mechanisms of antimicrobial peptide action and resistance. Pharmacol Rev 55(1):27-55.
18. Brown M R & Williams P (1985) Influence of substrate limitation and growth phase on sensitivity to antimicrobial agents. J Antimicrob Chemother 15 Suppl A:7-14.
19. Hein-Kristensen L, Knapp K M, Franzyk H, & Gram L (2013) Selectivity in the potentiation of antibacterial activity of alpha-peptide/beta-peptoid peptidomimetics and antimicrobial peptides by human blood plasma. Res Microbiol 164(9):933-940.
20. Bastos M D, Coutinho B G, & Coelho M L (2010) Lysostaphin: A Staphylococcal Bacteriolysin with Potential Clinical Applications. Pharmaceuticals (Basel) 3(4):1139-1161.
21. Kraus D & Peschel A (2008) Staphylococcus aureus evasion of innate antimicrobial defense. Future Microbiol 3(4):437-451.
22. Pruzanski W, Saito S, & Ogryzlo M A (1970) The significance of lysozyme (muramidase) in rheumatoid arthritis. I. Levels in serum and synovial fluid. Arthritis Rheum 13(4):389-399.
23. Wiegand et al. 2008. Agar and broth dilution methods to determine the minimal inhibitory concentration (MIC) of antimicrobial substances. Nature Protocols. 3:163-175.
24. Gilmer et al. 2013. Novel Bacteriophage Lysin with Broad Lytic Activity Protects against Mixed Infection by Streptococcus pyogenes and Methicillin-Resistant Staphylococcus aureus. Antimicrobial Agents and Chemotherapy. 57:2743-2750.
25. Hatful G. 2015. Dark Matter of the Biosphere: the Amazing World of Bacteriophage Diversity. Journal of Virology. 89:8107-10.
26. Fischetti, V. A., Nelson, D. & Schuch, R. 2006. Reinventing phage therapy: are the parts greater than the sum? Nature Biotechnology. 24:1508-11.
27. Kusuma & Kokai-Kun. 2005. Comparison of Four Methods for Determining Lysostaphin Susceptibility of Various Strains of Staphylococcus aureus. Antimicrobial Agents and Chemotherapy. 49:3256-63.
28. Methods for dilution antimicrobial susceptibility tests for bacteria that grow aerobically. Vol. 32 (Clinical and Laboratory Standards Institute (US), Wayne (Pa.), 2012). Clinical Microbiology Procedures Handbook 3rd Ed. Washington D.C., (ASM Press, 2010).
29. Fischetti, V. A. Bacteriophage lysins as effective antibacterials. Current opinion in microbiology 11, 393-400 (2008).
30. Fenton, M., Ross, P., McAuliffe, O., O'Mahony, J. & Coffey, A. Recombinant bacteriophage lysins as antibacterials. Bioengineered Bugs 1, 9-16 (2010).
31. Nelson, D., Loomis, L. & Fischetti, V. A. Prevention and elimination of upper respiratory colonization of mice by group A streptococci by using a bacteriophage lytic enzyme. Proceedings of the National Academy of Sciences of the United States of America 98, 4107-4112 (2001).
32. Witzenrath, M., et al. Systemic use of the endolysin Cp1-1 rescues mice with fatal pneumococcal pneumonia. Critical care medicine 37, 642-649 (2009).
33. Pastagia, M., et al. A novel chimeric lysin shows superiority to mupirocin for skin decolonization of methicillin-resistant and -sensitive Staphylococcus aureus strains. Antimicrobial agents and chemotherapy 55, 738-744 (2011).
34. Loeffler, J. M., Djurkovic, S. & Fischetti, V. A. Phage Lytic Enzyme Cp1-1 as a Novel Antimicrobial for Pneumococcal Bacteremia. Infection and Immunity 71, 6199-6204 (2003).
35. Schuch, R., Fischetti, V. A. & Nelson, D. C. A Genetic Screen to Identify Bacteriophage Lysins. in Bacteriophages: Methods and Protocols, Volume 2: Molecular and Applied Aspects, Vol. 502 307-319 (2009).
36. Bateman, A. & Rawlings, N. D. The CHAP domain: a large family of amidases including GSP amidase and peptidoglycan hydrolases. Trends Biochem Sci 28, 234-237 (2003).
37. Whisstock, J. C. & Lesk, A. M. SH3 domains in prokaryotes. Trends in Biochemical Sciences 24, 132-133 (1999).
38. Rossi, P., et al. Structural elucidation of the Cys-His-Glu-Asn proteolytic relay in the secreted CHAP domain enzyme from the human pathogen Staphylococcus saprophyticus. Proteins 74, 515-519 (2009).
39. Mueller, M., de la Pena, A. & Derendorf, H. Issues in Pharmacokinetics and Pharmacodynamics of Anti-Infective Agents: Kill Curves versus MIC. Antimicrobial agents and chemotherapy 48, 369-377 (2004).
40. Cottarel, G. & Wierzbowski, J. Combination drugs, an emerging option for antibacterial therapy. Trends in biotechnology 25, 547-555 (2007).
41. Tallarida, R. J. Revisiting the isobole and related quantitative methods for assessing drug synergism. The Journal of pharmacology and experimental therapeutics 342, 2-8 (2012).
42. LaPlante, K. L., Leonard, S. N., Andes, D. R., Craig, W. A. & Rybak, M. J. Activities of clindamycin, daptomycin, doxycycline, linezolid, trimethoprim-sulfamethoxazole, and vancomycin against community-associated methicillin-resistant Staphylococcus aureus with inducible clindamycin resistance in murine thigh infection and in vitro pharmacodynamic models. Antimicrobial agents and chemotherapy 52, 2156-2162 (2008).
43. Crandon, J. L., Kuti, J. L. & Nicolau, D. P. Comparative efficacies of human simulated exposures of telavancin and vancomycin against methicillin-resistant Staphylococcus aureus with a range of vancomycin MICs in a murine pneumonia model. Antimicrobial agents and chemotherapy 54, 5115-5119 (2010).
44. Loeffler, J. M., Nelson, D. & Fischetti, V. A. Rapid killing of Streptococcus pneumoniae with a bacteriophage cell wall hydrolase. Science 294, 2170-2172 (2001).
45. Schuch, R., Nelson, D. & Fischetti, V. A bacteriolytic agent that detects and kills Bacillus anthracis. Nature 418, 884-889 (2002).
46. Manoharadas, S., Witte, A. & Blasi, U. Antimicrobial activity of a chimeric enzybiotic towards Staphylococcus aureus. Journal of biotechnology 139, 118-123 (2009).
47. Rashel, M., et al. Efficient elimination of multidrug-resistant Staphylococcus aureus by cloned lysin derived from bacteriophage phi MR11. The Journal of infectious diseases 196, 1237-1247 (2007).
48. Daniel, A., et al. Synergism between a novel chimeric lysin and oxacillin protects against infection by methicillin-resistant Staphylococcus aureus. Antimicrobial agents and chemotherapy 54, 1603-1612 (2010).
49. Kokai-Kun, J. F., Chanturiya, T. & Mond, J. J. Lysostaphin as a treatment for systemic Staphylococcus aureus infection in a mouse model. The Journal of antimicrobial chemotherapy 60, 1051-1059 (2007).
50. Sopirala, M. M., et al. Synergy testing by Etest, microdilution checkerboard, and time-kill methods for pan-drug-resistant Acinetobacter baumannii. Antimicrobial agents and chemotherapy 54, 4678-4683 (2010).
51. Cassino C, Murphy G, Boyle J, Rotolo J, & Wittekind M (2016) Results of the First In Human Study of Lysin CF-301 Evaluating the Safety, Tolerability and Pharmacokinetic Profile in Healthy Volunteers. in ECCMID (Amsterdam, Netherlands).
52. CLSI (1999) Methods for Determining Bactericidal Activity of Antimicrobial Agents; Approved Guideline (Clinical and Laboratory Standards Institute, Wayne, Pa.).
53. Moody J (2010) Synergy testing: broth microdilution checkerboard and broth macrodilution methods. Clinical Microbiology Procedures Handbook, ed Garcia L S (ASM Press, Washington, D.C.), Vol 2, pp 5.12.11-15.12.23.
54. Pruzanski W, Saito S, & Ogryzlo M A (1970) The significance of lysozyme (muramidase) in rheumatoid arthritis. I. Levels in serum and synovial fluid. Arthritis Rheum 13(4):389-399.
55. Sahin O, Ziaei A, Karaismailoglu E, & Taheri N (2016) The serum angiotensin converting enzyme and lysozyme levels in patients with ocular involvement of autoimmune and infectious diseases. BMC Ophthalmol 16:19.
56. Georgieva T M, et al. (2008) Blood serum concentrations of total proteins and main protein fractions in weaning rabbits experimentally infected with E. coli. Revue de médecine vétérinaire 159(8-9):431-436.
57. Zaias J, Mineau M, Cray C, Yoon D, & Altman N H (2009) Reference values for serum proteins of common laboratory rodent strains. J Am Assoc Lab Anim Sci 48(4):387-390.
58. Finn T E, Nunez A C, Sunde M, & Easterbrook-Smith S B (2012) Serum albumin prevents protein aggregation and amyloid formation and retains chaperone-like activity in the presence of physiological ligands. J Biol Chem 287(25):21530-21540.
59. Sakoulas G, Eliopoulos G M, Alder J, & Eliopoulos C T (2003) Efficacy of daptomycin in experimental endocarditis due to methicillin-resistant Staphylococcus aureus. Antimicrob Agents Chemother 47(5):1714-1718
60. Nelson D, Schuch R, Chahales P, Zhu S, & Fischetti V A (2006) PlyC: a multimeric bacteriophage lysin. Proc Natl Acad Sci USA 103(28):10765-10770.
61. Schuch R, Pelzek A J, Kan S, & Fischetti V A (2010) Prevalence of Bacillus anthracis-like organisms and bacteriophages in the intestinal tract of Eisenia fetida earthworms. Applied & Environmental Microbiology 76:2286-2294.
62. CLSI (2015) Methods for Dilution Antimicrobial Susceptibility Tests for Bacteria That Grow Aerobically; Approved Standard-10th Edition (Clinical and Laboratory Standards Institute, Wayne, Pa.), CLSI document M07-A10.
63. Ghahramani P, et al. (2016) Pharmacokinetic Indices Driving Antibacterial Efficacy of CF-301—a Novel First-In-Class Lysin. in American Conference on Pharmacometrics (Bellevue, Wash.).
64. Chiu J. et al. (2016) Interspecies Scaling in Pre-clinical Population Pharmacokinetics of CF-301. in American Conference on Pharmacmetrics (Bellevue, Wash.).
65. Weidenmaier C. et al. (2005) DltABCD- and MprF-mediated cell envelope modifications of Staphylococcus aureus confer resistance to platelet microbicidal proteins and contribute to virulence in a rabbit endocarditis model. Infect Immun 73(12):8033-8038.
66. Verma P (2007) Methods for Determining Bactericidal Activity and Antimicrobial Interactions: Synergy Testing, Time-Kill Curves, and Population Analysis. Antimicrobial Susceptibility Testing Protocols, eds Schwalbe R, Steele-Moore L, & Goodwin A C (CRC Press, Boca Raton, Fla.), pp 275-298.

### Example 2

It is notable that the lysin polypeptides PlySs2 (CF-301), lysostaphin (LSP) and Sal lysins demonstrating the serum component effect, particularly via serum albumin and lysozyme, are similar in all having an SH3 type binding domain. To further evaluate the binding domain relevance to the serum component effect, various chimerics were constructed by replacing or swapping out one binding domain for another. Chimeric lysins having the Sal1 CHAP catalytic domain with ClyS binding domain, PlySs2 (CF-301) binding domain, or lysostaphin binding domain were assayed for MIC using MHB versus human serum. the results are depicted below in TABLE 14. The binding domain of ClyS does not support the blood effect, while the binding domain of PlySs2 (CF-301), which is an SH3 type binding domain, does. Similarly, the SH3 type binding domain of lysostaphin also demonstrates the serum component effect, retaining the serum effect when fused to the Sal1 catalytic domain as a chimeric lysin.

TABLE 14

| | Minimal inhibitors | | |
|---|---|---|---|
| | concentration (μg/mL) | | |
| | | Human | Fold decrease |
| Agent | MHB | serum | in MIC |
| PlySs2 (CF-301) | 32 | 1 | 32 |
| Lysostaphin | 2 | 0.0039 | 512 |
| ClyS | 8 | 4 | 2 |
| Sal1 | 2 | 0.06 | 32 |
| Sal1^{CHAP} + ClyS^{BD} | 64 | 64 | no |
| Sal1^{CHAP} + CF-301^{BD} | 2 | 0.125 | 16 |
| Sal1^{CHAP} + lysostaphin^{BD} | 2 | 0.25 | 8 |
| Daptomycin | 0.5 | 8 | no |
| Vancomycin | 1 | 1 | no |

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A composition for enhanced or synergistic killing of Gram-positive bacteria, the composition comprising an isolated lysin polypeptide having an SH3 type binding domain and one or more blood component protein, wherein the one or more blood component proteins comprise serum albumin, lysozyme or fragments thereof, wherein said fragments thereof demonstrate activity of the serum albumin or lysozyme protein with regard to enhancement or synergistic killing of the Gram-positive bacteria.

2. The composition of claim 1 wherein the lysin polypeptide having an SH3 type binding domain comprises PlySs2 lysin, Sal lysin, LysK lysin, lysostaphin, phill lysin, LysH5 lysin, MV-L lysin, LysGH15 lysin, or ALE-1 lysin.

3. The composition of claim 1 wherein the lysin polypeptide having an SH3 type binding domain is PlySs2 lysin and comprises an amino acid sequence of SEQ ID NO: 3 or a variant thereof having at least 80% identity to the amino acid sequence of SEQ ID NO: 3 and effective to kill Staphylococcus and Streptococcus bacteria.

4. The composition of claim 1 wherein the serum albumin comprises human serum albumin, horse serum albumin, dog serum albumin, rabbit serum albumin, rat serum albumin or calf serum albumin.

5. The composition of claim 1 wherein the lysozyme is human lysozyme.

6. The composition of claim 1 wherein the one or more blood component proteins has no or limited intrinsic antibacterial activity, in the absence of the lysin polypeptide.

7. The composition of claim 1 further comprising one or more serum fatty acids.

8. The composition of claim 7 wherein the serum fatty acids comprise oleate or palmitate.

9. A method of killing or reducing a population of Gram-positive bacteria comprising contacting the Gram-positive bacteria with a composition comprising an amount of an isolated lysin polypeptide effective to kill the Gram-positive bacteria, the isolated lysin polypeptide having an SH3-type binding domain, and one or more blood component proteins selected from serum albumin, lysozyme and fragments thereof.

10. The method of claim 9 wherein the serum albumin comprises human serum albumin, horse serum albumin, dog serum albumin, rabbit serum albumin, rat serum albumin or calf serum albumin.

11. The method of claim 9 wherein the serum albumin is human serum albumin, or a fragment thereof capable of binding Gram-positive bacteria.

12. The method of claim 9 wherein the lysozyme is human lysozyme.

13. The method of claim 9 wherein the Gram-positive bacteria is Staphylococcus or Streptococcus bacteria.

14. The method of claim 9 wherein the Gam-positive bacteria is Staphylococcus aureus.

15. The method of claim 9 wherein the lysin polypeptide having an SH3 type binding domain comprises PlySs2 lysin, Sal lysin, LysK lysin, lysostaphin, phill lysin, LysH5 lysin, MV-L lysin, LysGH15 lysin, or ALE-1 lysin.

16. The method of claim 9 wherein the lysin polypeptide having an SH3 type binding domain is PlySs2 lysin and comprises an amino acid sequence of SEQ ID NO: 3 or a variant thereof having at least 80% identity to the amino acid sequence of SEQ ID NO: 3 and effective to kill Staphylococcus and Streptococcus bacteria.

17. The method of claim 9 wherein the bacteria is further contacted with a serum fatty acid.

18. A method for treating an antibiotic-resistant Staphylococcus aureus infection in a human comprising administering to a human having an antibiotic-resistant Staphylococcus aureus infection, an effective amount of the composition of claim 1.

19. A method for treating an antibiotic-resistant Staphylococcus aureus infection in a human comprising administering to a human having an antibiotic-resistant Staphylococcus aureus infection, an effective amount of a composition comprising an amount of an isolated lysin polypeptide having an SH3-type binding domain and capable of killing a Gram-positive bacteria, and human lysozyme.

20. The method of claim 19, further comprising evaluating a level of human serum albumin at the site of infection and/or evaluating a coating of the antibiotic-resistant Staphylococcus aureus at the site of infection with human serum albumin, and administering human serum albumin or a fragment thereof capable of binding Gram-positive bacteria, whereby the lysin polypeptide and human lysozyme are effective to kill the antibiotic-resistant Staphylococcus aureus.

21. A chimeric or fusion polypeptide comprising a lysin-derived SH3-type bacterial binding domain and human serum albumin or a fragment thereof capable of binding Gram-positive bacteria.

22. The chimeric or fusion polypeptide of claim 21 further comprising a lytic domain of human lysozyme.

23. A chimeric or fusion polypeptide comprising a lysin-derived SH3-type bacterial binding domain and human lysozyme or a fragment thereof capable of lysing Gram-positive bacteria.

24. The chimeric or fusion polypeptide of claim 23 further comprising human serum albumin or a fragment thereof capable of binding Gram-positive bacteria.

25. A method for enhancing the antibacterial activity of an antibacterial agent or peptide comprising administering the agent or peptide in combination with a lysin polypeptide having an SH3-type binding domain and human serum albumin or a fragment thereof capable of binding Gram-positive bacteria.

26. The method of claim 25 further comprising administering human lysozyme or a fragment thereof capable of lysing Gram-positive bacteria.

27. A method for enhancing the antibacterial activity of an antibacterial agent or peptide comprising administering the agent or peptide in combination with a lysin polypeptide having an SH3-type binding domain and lysozyme or a fragment thereof capable of lysing Gram-positive bacteria.

28. The method of claim 27 further comprising administering human serum albumin or a fragment thereof capable of binding Gram-positive bacteria.

29. A method for susceptibility testing of Gram-positive bacteria comprising evaluating an antibacterial peptide in broth, assay medium or solution supplemented with serum albumin.

30. The method of claim 29 wherein the broth, assay medium or solution is supplemented with human serum albumin, horse serum albumin, dog serum albumin, rabbit serum albumin, rat serum albumin or calf serum albumin.

31. The method of claim 29 wherein the broth, assay medium or solution is supplemented with human serum albumin, horse serum albumin, dog serum albumin or rabbit serum albumin.

32. The method of claim 29 wherein the broth, assay medium or solution is supplemented with lysozyme.

33. The method of claim 29 wherein the broth, assay medium or solution is supplemented with human serum albumin at a concentration between 10% and 50% human serum albumin.

34. The method of claim 29 wherein the broth, assay medium or solution is supplemented with human serum albumin at a concentration between 20% and 40% human serum albumin.

35. The method of any claim 29 wherein the antibacterial peptide is a lysin polypeptide having an SH3 binding domain.

36. The method of claim 35 wherein the lysin polypeptide comprises PlySs2 lysin, Sal lysin, LysK lysin, lysostaphin, phill lysin, LysH5 lysin, MV-L lysin, LysGH15 lysin, or ALE-1 lysin.

37. The method of claim 35 wherein the lysin polypeptide is PlySs2 and comprises an amino acid sequence of SEQ ID NO: 3 or a variant thereof having at least 80% identity to the amino acid sequence of SEQ ID NO: 3 and effective to kill Staphylococcus and Streptococcus bacteria.

38. The method of claim 29 for evaluating a composition comprising an antibacterial peptide which is a lysin polypeptide and further comprising one or more antibacterial agent.

39. The method of claim 38 wherein the one or more antibacterial agent is an antibiotic.
